SEQUENCE LISTING (65 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 4 - protein, 107 aa, synthetic construct
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, synthetic construct
RASKSINKYLA

SEQ ID NO: 6 - protein, 7 aa, synthetic construct
SGSTLQS

SEQ ID NO: 7 - protein, 9 aa, synthetic construct
QQHNEYPLT

SEQ ID NO: 8 - protein, 121 aa, synthetic construct
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, synthetic construct
GYHFTSYWMH

SEQ ID NO: 10 - protein, 17 aa, synthetic construct
VIHPNSGSINYNEKFES

SEQ ID NO: 11 - protein, 12 aa, synthetic construct
ERDSTEVLPMDY

SEQ ID NO: 12 - DNA, 321 bp, synthetic construct
  source 1..321: mol_type = other DNA
gatgtccagataacccagtctccatcttatcttgctgcatctcctggagaaaccattactattaattgcagggcaagtaagagcattaacaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacaacataatgaatacccgctcacgttcggtgctgggaccaagctggagctgaaa

SEQ ID NO: 13 - DNA, 363 bp, synthetic construct
  source 1..363: mol_type = other DNA
caggtccaactgcagcagcctggggctgagctggtaaagcctggggcttcagtgaagttgtcctgcaagtcttctggctaccatttcaccagctactggatgcactgggtgaagcagaggcctggacaaggccttgagtggattggagtgattcatcctaatagtggtagtattaactacaatgagaagttcgagagcaaggccacactgactgtagacaaatcctccagcacagcctacatgcaactcagcagcctgacatctgaggactcggcggtctattattgtgcaggagagagagattctacggaggttctccctatggactactggggtcaaggaacctcagtcaccgtctcctca

SEQ ID NO: 14 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 121 aa, synthetic construct; identical to SEQ ID NO: 8

SEQ ID NO: 16 - protein, 121 aa, synthetic construct [sequence omitted]

SEQ ID NO: 17 - protein, 121 aa, synthetic construct [sequence omitted]

SEQ ID NO: 18 - protein, 121 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 19 - protein, 121 aa, synthetic construct [sequence omitted]

SEQ ID NO: 20 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 451 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 115 aa, synthetic construct
QVKLEESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVT

SEQ ID NO: 23 - protein, 109 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 24 - protein, 118 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSS

SEQ ID NO: 25 - protein, 216 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 445 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 12 aa, synthetic construct
TASSSVSSSYLH

SEQ ID NO: 28 - protein, 7 aa, synthetic construct
STSNLAS

SEQ ID NO: 29 - protein, 5 aa, synthetic construct
NYAMS

SEQ ID NO: 30 - protein, 17 aa, synthetic construct
TISSGGSHTYYLDSVKG

SEQ ID NO: 31 - protein, 121 aa, synthetic construct [sequence omitted]

SEQ ID NO: 32 - protein, 121 aa, synthetic construct [sequence omitted]

SEQ ID NO: 33 - protein, 121 aa, synthetic construct; identical to SEQ ID NO: 18

SEQ ID NO: 34 - protein, 121 aa, synthetic construct [sequence omitted]

SEQ ID NO: 35 - protein, 107 aa, synthetic construct [sequence omitted]

SEQ ID NO: 36 - protein, 107 aa, synthetic construct [sequence omitted]

SEQ ID NO: 37 - protein, 107 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, synthetic construct [sequence omitted]

SEQ ID NO: 39 - protein, 229 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 43 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 45 - protein, 448 aa, synthetic construct
QVQLVQSGAELKKPGASVKVSCKSSGYHFTSYWMKWVKQAPGQGLEWIGVIHPNSGSINYNKKFESRVTITVDKSTSTAYMELSSLRSEDTAVYYCAGARKSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 46 - protein, 448 aa, synthetic construct
QVQLVQSGAELKKPGASVKVSCKSSGYHFTSYWMHWVKQAPGQGLEWIGVIHPNSGSINYNKKFKSRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 47 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 49 - protein, 448 aa, synthetic construct
QVQLVQSGAELKKPGASVKVSCKSSGYHFTSYWMKWVKQAPGQGLEWIGVIHPNSGSINYNKKFESRVTITVDKSTSTAYMELSSLRSEDTAVYYCAGARKSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 50 - protein, 448 aa, synthetic construct
QVQLVQSGAELKKPGASVKVSCKSSGYHFTSYWMHWVKQAPGQGLEWIGVIHPNSGSINYNKKFKSRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 51 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGARASTAVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 52 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 53 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 54 - protein, 12 aa, synthetic construct
SSVSSSYLHWYQ

SEQ ID NO: 55 - protein, 10 aa, synthetic construct
STSNLASGVP

SEQ ID NO: 56 - protein, 10 aa, synthetic construct
HQYYRLPPIT

SEQ ID NO: 57 - protein, 12 aa, synthetic construct
GFTFSNYAMSWV

SEQ ID NO: 58 - protein, 10 aa, synthetic construct
ISSGGSHTYY

SEQ ID NO: 59 - protein, 11 aa, synthetic construct
ARLFTGYAMDY

SEQ ID NO: 60 - protein, 109 aa, synthetic construct
DIVMTQTTAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 61 - protein, 9 aa, synthetic construct
LFTGYAMDY

SEQ ID NO: 62 - protein, 109 aa, synthetic construct
QIVLTQSPAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 63 - protein, 118 aa, synthetic construct
EVMLVESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVTVSS

SEQ ID NO: 64 - protein, 330 aa, synthetic construct [sequence omitted]

SEQ ID NO: 65 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGARASTAVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

(19) United States
(12) Patent Application Publication
(10) Pub. No.: US 2025/0313615 A1
(43) Pub. Date: Oct. 9, 2025

(54) COMPOSITIONS AND METHODS FOR TREATING LUPUS NEPHRITIS

(71) Applicant: Annexon, Inc., Brisbane, CA (US)

(72) Inventors: Ann Mongan, Brisbane, CA (US); Henk-Andre' Kroon, Brisbane, CA (US); Ellen Cahir-McFarland, Brisbane, CA (US); Ted Yednock, Forest Knolls, CA (US); Dean R. Artis, Kensington, CA (US); Yaisa Andrews-Zwilling, Brisbane, CA (US)

(21) Appl. No.: 18/865,443

(22) PCT Filed: May 15, 2023
(86) PCT No.: PCT/US23/66993

§ 371 Date: Nov. 13, 2024

Related U.S. Application Data
(60) Provisional application No. 63/341,835, filed on May 13, 2022.

(51) Int. Cl. C07K 16/18; A61K 38/17

(52) U.S. Cl. CPC C07K 16/18; A61K 38/1725; C07K 2317/24; C07K 2317/31

(57) ABSTRACT

The present disclosure relates generally to methods of treating lupus nephritis in a subject in need thereof. The method comprises determining that the subject has at least one of the following characteristics: an elevated C4x level; an elevated C4x/C4 ratio, a reduced C4 level; an elevated C1sC1 inhibitor level; an elevated C1sC1 inhibitor/Cis ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level; wherein C4x is selected from C4a, C4b and C4d, and administering to the subject an inhibitor of the classical complement pathway.

### RELATED APPLICATIONS

This patent application claims priority to U.S. Provisional Patent Application No. 63/341,835, filed May 13, 2022, which is hereby incorporated by reference in its entirety.

### BACKGROUND

Systemic lupus erythematosus (SLE) is a biologically and clinically heterogeneous autoimmune disease that is characterized by a broad spectrum of diverse clinical involvement, particularly kidney involvement. SLE is an autoimmune disease in which the body's immune system mistakenly attacks healthy tissue in many parts of the body. Symptoms vary among people and may be mild to severe. Common symptoms include painful and swollen joints, fever, chest pain, hair loss, mouth ulcers, swollen lymph nodes, feeling tired, and a rash which is most commonly on the face. Often there are periods of illness, called flares, and periods of remission during which there are few symptoms.

The cause of SLE is not clear. It is thought to involve genetics together with environmental factors. There are a number of other kinds of lupus erythematosus including discoid lupus erythematosus, neonatal lupus, and subacute cutaneous lupus erythematosus. Renal involvement in SLE is termed lupus nephritis (LN). LN occurs in up to 50% to 60% of SLE patients within the first 10 years of the disease and is a major cause of morbidity and mortality in these patients. To date, there is still no cure for lupus nephritis. There are only options to help control symptoms, to prevent flare ups, and reduce organ damage. Therefore, there is a need in the art for new therapies to prevent and treat lupus nephritis.

### SUMMARY

The present disclosure is generally directed to methods of treating lupus nephritis in a subject in need thereof. The method comprises determining that the subject has at least one of the following characteristics: an elevated C4x level; an elevated C4x/C4 ratio; a reduced C4 level; an elevated C1sC1 inhibitor level, an elevated C1sC1 inhibitor/Cis ratio; a reduced C1 s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. C4x may be selected from the group consisting of C4a, and C4d. The method further comprises administering to the subject an inhibitor of the classical complement pathway, e.g., if the subject has an elevated C4x level, an elevated C4x/C4 ratio, a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated PACA1 and/or PACA3 level. A therapeutically effective amount of the inhibitor may be administered.

In some embodiments, the subject has at least two of the characteristics. For example, the characteristics may be selected from elevated C4x levels, elevated C4x/C4 ratio and reduced C4 levels, and/or the characteristics may be selected from the group consisting of elevated C1sC1 inhibitor levels, elevated C1sC1 inhibitor/Cis ratio and reduced C1s levels.

In some embodiments, the subject further has an elevated Pathogenic Anti-C1q Antibody 1 (PACA1) and/or Pathogenic Anti-C1q Antibody 3 (PACA3) level. In some embodiments, the subject has at least one of the following additional characteristics: an elevated C3a level; an elevated C3a/C3 ratio; or a reduced C3 level.

In some embodiments, the subject has an elevated C4x level. In some embodiments, the subject further has at least one of the following additional characteristics: an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level.

In some embodiments, the elevated C4x level is greater than a C4x level in normal or healthy subjects, is greater than a C4x level in normal or healthy subjects of a similar age, or is greater than a reference C4x level. The reference C4x level may be a value that is equal to or greater than the mean or median of C4x levels in samples derived from lupus nephritis subjects, or a value that is equal to or greater than the mean or median of C4x levels in samples derived from lupus nephritis subjects of a similar age. The reference C4x level may be a value that is equal to or greater than the 55th percentile, 60th percentile, 65th percentile, 70th percentile, 75th percentile, 80th percentile, 85th percentile, 90th percentile, 95th percentile, or 100th percentile of C4x levels in samples derived from lupus nephritis subjects. The reference C4x level may be a value that is equal to or greater than the 55th percentile, 60th percentile, 65th percentile, 70th percentile, 75th percentile, 80th percentile, 85th percentile, 90th percentile, 95th percentile, or 100th percentile of (4x levels in samples derived from lupus nephritis subjects of a similar age. The reference C4x level may be a value that is equal to or greater than the 55th, 60th, 65th, 70th, 75th, 80th, 85th, 90th, 95th, or 100th percentile of C4x levels in samples derived from normal or healthy subjects. The reference C4x level may be a value that is equal to or greater than the 55th, 60th, 65th, 70th, 75th, 80th, 85th, 90th, 95th, or 100th percentile of C4x levels in samples derived from normal or healthy subjects of a similar age. In some embodiments, the normal or healthy subjects do not have lupus nephritis. In some embodiments, the elevated C4x level is greater than the reference C4x level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%, or the elevated C4x level is greater than the reference C4x level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C4x/C4 ratio. In some embodiments, the subject further has at least one of the following additional characteristics: an elevated C4x level; a reduced C4 level; an elevated C1sC1 inhibitor level; an elevated C1sC1 inhibitor/C Is ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C4x/C4 ratio is greater than a C4x/C4 ratio in normal or healthy subjects, is greater than a C4x/C4 ratio in normal or healthy subjects of a similar age, or is greater than a reference C4x/C4 ratio The reference C4x/C4 ratio may be a value that is equal to or greater than the mean or median of C4x/C4 ratio in samples derived from lupus nephritis subjects, or may be equal to or greater than the mean or median of C4x/C4 ratio in samples derived from lupus nephritis subjects of a similar age. The reference C4x/C4 ratio may be a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects. The reference C4x/C4 ratio may be a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects of a similar age. The reference C4x/C4 ratio may be a value that is equal to or greater than the 55th, 60th, 65th, 70th, 75th, 80th, 85th, 90th, 95th, or 100th percentile of C4x/C4 ratio in samples derived from normal or healthy subjects. The reference C4x/C4 ratio may be a value that is equal to or greater than the 55th, 60th, 65th, 70th, 75th, 80th, 85th, 90th, 95th, or 100th percentile of C4x/C4 ratio in samples derived from normal or healthy subjects of a similar age. In some embodiments, the normal or healthy subjects do not have lupus nephritis. In some embodiments, the elevated C4x/C4 ratio is greater than the reference C4x/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C4x/C4 ratio by at least 1%-10%, 10%-20%, 20%-30%, 30%-40%, 40%-50%, 50%-60%, 60%-70%, 70%-80%, 80%-90%, 90%-100%, 100%-200/0, 200%-300%, 300%-400%, or 400%-500%.

In some embodiments, the subject has a reduced C4 level. In some embodiments, the subject further has at least one of the following additional characteristics: an elevated C4x level; an elevated C4x/C4 ratio; an elevated C1sC1 inhibitor level; an elevated C1sC1 inhibitor/C1s ratio; a reduced C1s level; an elevated C2b level; an elevated C2b/C2 ratio;
a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C4 level is less than a C4 level in normal or healthy subjects. The reduced C4 level may be less than a C4 level in normal or healthy subjects of a similar age, may be less than a reference C4 level, or may be a value that is equal to or less than the mean or median of C4 levels in samples derived from lupus nephritis subjects. In some embodiments, the reference C4 level is a value that is equal to or less than the mean or median of C4 levels in samples derived from lupus nephritis subjects of a similar age. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects of a similar age. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects of a similar age. In some embodiments, the normal or healthy subjects do not have lupus nephritis. In some embodiments, the reduced C4 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C1sC1 inhibitor level. In some embodiments, the subject has at least one of the following additional characteristics: an elevated C4x level; an elevated C4x/C4 ratio; a reduced C4 level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C1sC1 inhibitor level is greater than a C1sC1 inhibitor level in normal or healthy subjects, is greater than a C1sC1 inhibitor level in normal or healthy subjects of a similar age, or is greater than a reference C1sC1 inhibitor level. The reference C1sC1 inhibitor level may be a value that is equal to or greater than the mean or median of C1sC1 inhibitor levels in samples derived from lupus nephritis subjects, or may be a value that is equal to or greater than the mean or median of C1sC1 inhibitor levels in samples derived from lupus nephritis subjects of a similar age. The reference C1sC1 inhibitor level may be a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects. The reference C1sC1 inhibitor level may be a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects of a similar age. The reference C1sC1 inhibitor level may be a value that is equal to or greater than the 55th, 60th, 65th, 70th, 75th, 80th, 85th, 90th, 95th, or 100th percentile of C1sC1 inhibitor levels in samples derived from normal or healthy subjects. The reference C1sC1 inhibitor level may be a value that is equal to or greater than the 55th, 60th, 65th, 70th, 75th, 80th, 85th, 90th, 95th, or 100th percentile of C1sC1 inhibitor levels in samples derived from normal or healthy subjects of a similar age. In some embodiments, the normal or healthy subjects do not have lupus nephritis. In some embodiments, the elevated C1sC1 inhibitor level is greater than the reference C1sC1 inhibitor level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C1sC1 inhibitor level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C1sC1 inhibitor/C1s ratio. In some embodiments, the subject has at least one of the following additional characteristics: an elevated C4x level; a reduced C4 level; an elevated C4x/C4 ratio; an elevated C1sC1 inhibitor level; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C1sC1 inhibitor/C1s ratio is greater than a C1sC1 inhibitor/C1s ratio in normal or healthy subjects. In some embodiments, the elevated C1sC1 inhibitor/C1s ratio is greater than a C1sC1 inhibitor/C1s ratio in normal or healthy subjects of a similar age. In some embodiments, the elevated C1sC1 inhibitor/C1s ratio is greater than a reference C1sC1 inhibitor/C1s ratio. In some embodiments, the reference C1sC1 inhibitor/C1s ratio is a value that is equal to or greater than the mean or median of C1sC1 inhibitor/C1s ratio in samples derived from lupus nephritis subjects. In some embodiments, the reference C1sC1 inhibitor/C1s ratio is a value that is equal to or greater than the mean or median of C1sC1 inhibitor/C1s ratio in samples derived from lupus nephritis subjects of a similar age. In some embodiments, the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from lupus nephritis subjects. In some embodiments, the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from lupus nephritis subjects of a similar age. In some embodiments, the reference C1sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C Is ratio in samples derived from normal or healthy subjects. In some embodiments, the reference C1 sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from normal or healthy subjects of a similar age. In some embodiments, the normal or healthy subjects do not have lupus nephritis. In some embodiments, the elevated C1sC1 inhibitor/C1s ratio is greater than the reference C1sC1 inhibitor/C1 s ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C1sC1 inhibitor/C1s ratio is greater than the reference C1sC1 inhibitor/C1s ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C1s level. In some embodiments, the subject has at least one of the following additional characteristics; an elevated C4x level; an elevated C4x/C4 ratio; a reduced C4 level; an elevated C1sC1 inhibitor/C1s ratio; an elevated C1sC1 inhibitor level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C1s level is less than a C1s level in normal or healthy subjects. In some embodiments, the reduced C is level is less than a C1s level in normal or healthy subjects of a similar age. In some embodiments, the reduced C1s level is less than a reference C1s level. In some embodiments, the reference C1s level is a value that is equal to or less than the mean or median of C1s levels in samples derived from lupus nephritis subjects. In some embodiments, the reference C1s level is a value that is equal to or less than the mean or median of C1s levels in samples derived from lupus nephritis subjects of a similar age. In some embodiments, the reference C1 s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s level in samples derived from lupus nephritis subjects. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1 s level in samples derived from lupus nephritis subjects of a similar age. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects of a similar age. In some embodiments, the normal or healthy subjects do not have lupus nephritis. In some embodiments, the reduced C1s level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the reduced C1s level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C2b level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; a reduced C1s level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C2b level is greater than a C2b level in normal or healthy subjects, the elevated C2b level is greater than a C2b level in normal or healthy subjects of a similar age, or is greater than a reference C2b level. The reference C2b level may be a value that is equal to or greater than the mean or median of C2b levels in samples derived from lupus nephritis subjects, or is a value that is equal to or greater than the mean or median of C2b levels in samples derived from lupus nephritis subjects of a similar age. The reference C2b level may be a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects. The reference C2b level may be a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects of a similar age. The reference C2b level may be a value that is equal to or greater than the 55th, 60th, 65th, 70th, 75th, 80th, 85th, 90th, 95th, or 100th percentile of C2b levels in samples derived from normal or healthy subjects. The reference C2b level may be a value that is equal to or greater than the 55th, 60th, 65th, 70th, 75th, 80th, 85th, 90th, 95th, or 100th percentile of C2b levels in samples derived from normal or healthy subjects of a similar age. In some embodiments, the normal or healthy subjects do not have lupus nephritis. In some embodiments, the elevated C2b level is greater than the reference C2b level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C2b level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C2b/C2 ratio. In some embodiments, the subject has at least one of the following additional characteristics: an elevated C4x level; an elevated C4x/C4 ratio; reduced C4 level; an elevated C1sC1 inhibitor/C1s ratio; an elevated C1sC1 inhibitor level; a reduced C1s level; a reduced C2 level; an elevated C2b level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C2b/C2 ratio is greater than a C2b/C2 ratio in normal or healthy subjects. In some embodiments, the elevated C2b/C2 ratio is greater than a C2b/C2 ratio in normal or healthy subjects of a similar age. In some embodiments, the elevated C2b/C2 ratio is greater than a reference C2b/C2 ratio. In some embodiments, the reference C2b/C2 ratio is a value that is equal to or greater than the mean or median of C2b/C2 ratio in samples derived from lupus nephritis subjects. In some embodiments, the reference C2b/C2 ratio is a value that is equal to or greater than the mean or median of C2b/C2 ratio in samples derived from lupus nephritis subjects of a similar age. In some embodiments, the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects. In some embodiments, the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects of a similar age. In some embodiments, the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects. In some embodiments, the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects of a similar age. In some embodiments, the normal or healthy subjects do not have lupus nephritis. In some embodiments, the elevated C2b/C2 ratio is greater than the reference C2b/C2 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C2b/C2 ratio is greater than the reference C2b/C2 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C2 level. In some embodiments, the subject has at least one of the following additional characteristics: an elevated (4x level; an elevated C4x/C4 ratio; a reduced C4 level, an elevated C1sC1 inhibitor/C1s ratio; an elevated C1sC1 inhibitor level; a reduced C1s level; an elevated C2b level; an elevated C2b/C2 ratio; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C2 level is less than a C2 level in normal or healthy subjects, is less than a C2 level in normal or healthy subjects of a similar age, or is less than a reference C2 level. The reference C2 level may be a value that is equal to or less than the mean or median of C2 levels in samples derived from lupus nephritis subjects or may be a value that is equal to or less than the mean or median of C2 levels in samples derived from lupus nephritis subjects of a similar age. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects of a similar age. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects or may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects of a similar age. In some embodiments, the normal or healthy subjects do not have lupus nephritis. In some embodiments, the reduced C2 level is less than the reference C2 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is less than the reference C2 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated Pathogenic Anti-C1q Antibody 1 (PACA1) and/or Pathogenic Anti-C1q Antibody 3 (PACA3) level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; a reduced C1s level; an elevated C2b level; an elevated C2b/C2 ratio; a reduced C2 level; an elevated C3a level; an elevated C3a/C3 ratio; or a reduced C3 level.

In some embodiments, the elevated PACA1 and/or PACA3 level is greater than a PACA1 and/or PACA3 level in normal or healthy subjects, is greater than a PACA1 and/or PACA3 level in normal or healthy subjects of a similar age, or is greater than a reference PACA1 and/or PACA3 level. The reference PACA1 and/or PACA3 level may be a value that is equal to or greater than the mean or median of PACA1 and/or PACA3 levels in samples derived from lupus nephritis subjects or may be a value that is equal to or greater than the mean or median of PACA1 and/or PACA3 levels in samples derived from lupus nephritis subjects of a similar age. The reference PACA1 and/or PACA3 level may be a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects. The reference PACA1 and/or PACA3 level may be a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects of a similar age. The reference PACA1 and/or PACA3 level may be a value that is equal to or greater than the 55th, 60th, 65th, 70th, 75th, 80th, 85th, 90th, 95th, or 100th percentile of PACA1 and/or PACA3 levels in samples derived from normal or healthy subjects or may be a value that is equal to or greater than the 55th, 60th, 65th, 70th, 75th, 80th, 85th, 90th, 95th, or 100th percentile of PACA1 and/or PACA3 levels in samples derived from normal or healthy subjects of a similar age. In some embodiments, the normal or healthy subjects do not have lupus nephritis. In some embodiments, the elevated PACA1 and/or PACA3 level is greater than the reference PACA1 and/or PACA3 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference PACA1 and/or PACA3 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C3a level. In some embodiments, the subject has at least one of the following additional characteristics: an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio, a reduced C2 level; an elevated Pathogenic Anti-C1q Antibody 1 (PACA1) and/or Pathogenic Anti-C1q Antibody 3 (PACA3) level; an elevated C4x level; an elevated C3a/C3 ratio; or a reduced C3 level. In some embodiments, the elevated C3a level is greater than a C3a level in normal or healthy subjects. In some embodiments, the elevated C3a level is greater than a C3a level in normal or healthy subjects of a similar age. In some embodiments, the elevated C3a level is greater than a reference C3a level. In some embodiments, the reference C3a level is a value that is equal to or greater than the mean or median of C3a levels in samples derived from lupus nephritis subjects. In some embodiments, the reference C3a level is a value that is equal to or greater than the mean or median of C3a levels in samples derived from lupus nephritis subjects of a similar age. In some embodiments, the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects. In some embodiments, the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects of a similar age. In some embodiments, the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects. In some embodiments, the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects of a similar age. In some embodiments, the normal or healthy subjects do not have lupus nephritis. In some embodiments, the elevated C3a level is greater than the reference C3a level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C3a level is greater than the reference C3a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C3a/C3 ratio. In some embodiments, the subject has at least one of the following additional characteristics: an elevated C4x level; a reduced C4 level; an elevated C1sC1 inhibitor level; an elevated C1sC1 inhibitor/C Is ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C4x/C4 ratio; or a reduced C3 level. In some embodiments, the elevated C3a/C3 ratio is greater than a C3a/C3 ratio in normal or healthy subjects. In some embodiments, the elevated C3a/C3 ratio is greater than a C3a/C3 ratio in normal or healthy subjects of a similar age. In some embodiments, the elevated C3a/C3 ratio is greater than a reference C3a/C3 ratio. In some embodiments, the reference C3a/C3 ratio is a value that is equal to or greater than the mean or median of C3a/C3 ratio in samples derived from lupus nephritis subjects. In some embodiments, the reference C3a/C3 ratio is a value that is equal to or greater than the mean or median of C3a/C3 ratio in samples derived from lupus nephritis subjects of a similar age. In some embodiments, the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects. In some embodiments, the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects of a similar age. In some embodiments, the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects. In some embodiments, the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects of a similar age. In some embodiments, the normal or healthy subjects do not have lupus nephritis. In some embodiments, the elevated C3a/C3 ratio is greater than the reference C3a/C3 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C3a/C3 ratio is greater than the reference C3a/C3 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C3 level. In some embodiments, the subject has at least one of the following additional characteristics: an elevated C4x level; an elevated C4x/C4 ratio; an elevated C1sC1 inhibitor level; an elevated C1sC1 inhibitor/C1s ratio; a reduced C Is level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C4 level. In some embodiments, the reduced C3 level is less than a C3 level in normal or healthy subjects. In some embodiments, the reduced C3 level is less than a C3 level in normal or healthy subjects of a similar age. In some embodiments, the reduced C3 level is less than a reference C3 level. In some embodiments, the reference C3 level is a value that is equal to or less than the mean or median of C3 levels in samples derived from lupus nephritis subjects. In some embodiments, the reference C3 level is a value that is equal to or less than the mean or median of C3 levels in samples derived from lupus nephritis subjects of a similar age. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects of a similar age. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects of a similar age. In some embodiments, the normal or healthy subjects do not have lupus nephritis. In some embodiments, the reduced C3 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the reduced C3 level is less than the reference C4 level by at least %-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the C4x level, or the C4x/C4 ratio, or the C4 level, or the C1sC1 inhibitor level, or the C1sC1 inhibitor/C1s ratio, or the C1 s level, or the C2b level, or the C2b/C2 ratio, or the C2 level, or the PACA1 and/or PACA3 level, or the C3a level, or the C3a/C3 ratio, or the C3 level is measured in a biological sample. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured in plasma or urine. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 12 months before the administration of a therapeutically effective amount of an inhibitor of the classical complement pathway. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 6 months before the administration of an inhibitor. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 3 months before the administration of an inhibitor. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 1 month before the administration of an inhibitor.

In some embodiments, the subject has an elevated urine protein/creatinine ratio (UPCR) level. In some embodiments, the elevated UPCR level is greater than a UPCR level in normal or healthy subjects, is greater than a UPCR level in normal or healthy subjects of a similar age, or is greater than a reference UPCR level. The reference UPCR level may be equal to or greater than about 0.5 g/g, 1.0 g/g, 1.5 g/g, 2.0 g/g, 2.5 g/g, 3.0 g/g, 3.5 g/g, 4.0 g/g, 4.5 g/g, or 5.0. The elevated UPCR level may be greater than the UPCR level in normal or healthy subjects or the reference UPCR level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000%, 4000%, or 5000%. The elevated UPCR level may be greater than the UPCR level in normal or healthy subjects or the reference UPCR level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%. In some embodiments, the UPCR level is measured in urine.

In some embodiments, the classical complement inhibitor is a C1 inhibitor, such as a small molecule, an antibody, an aptamer, an antisense nucleic acid or a gene editing agent. In some embodiments, the antibody is a monoclonal antibody, a polyclonal antibody, a recombinant antibody, a humanized antibody, a chimeric antibody, a multispecific antibody, or an antibody derivative thereof.

In some embodiments, the inhibitor of the classical complement pathway is a C1q inhibitor, such as a small molecule, an antibody, an aptamer, an antisense nucleic acid or a gene-editing agent. In some embodiments, the antibody is a monoclonal antibody, a polyclonal antibody, a recombinant antibody, a humanized antibody, a chimeric antibody, a multispecific antibody, or an antibody derivative thereof. The antibody may be an anti-C1q antibody. In some embodiments, the antibody derivative thereof is a single arm antibody.

In some embodiments, the antibody is administered at a dose of at least 50 mg/kg, or at a dose between 50 mg/kg to 200 mg/kg. The antibody may be administered at a dose of 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, or 200 mg/kg. In some embodiments, the antibody is administered to a total of at least 50 mg. The antibody may be administered to a total dose of 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the antibody is administered daily, once a week, once every other week, once a month, once every six weeks, or once every other month. In some embodiments, the antibody is administered for at least 3 months, 6 months, at least 7 months, at least 8 months, at least 9 months, at least 10 months, at least 11 months, or at least 12 months, preferably for at least 6 months. The antibody may be administered throughout lifetime of the patient or intermittently throughout the lifetime of the patient, e.g., in response to a flare. In some embodiments, the antibody is administered at a dose of 75 mg/kg on day 1 and on day 5 or day 6. In some embodiments, the antibody is further administered at a dose of 100 mg/kg every two weeks. In some embodiments, the antibody is administered intravenously.

In some embodiments, the antibody is an antibody fragment, such as a Fab fragment, a Fab′ fragment, a F(ab′)2 fragment, a Fv fragment, a diabody, or a single chain antibody molecule. In some embodiments, the antibody fragment is administered to a total dose of at least 250 mg or to a total dose between 250 mg to 1000 mg. In some embodiments, the antibody fragment is administered to a total dose of 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg. In some embodiments, the antibody fragment is administered at a total dose of about 750 mg. In some embodiments, the antibody fragment is administered daily, once every other day, once every three days, once every four days, once every five days, or once every six days, once a week, once every two weeks, or once a month. In some embodiments, the antibody fragment is administered three times per week. In some embodiments, the antibody fragment is administered for at least 3 months, 6 months, at least 7 months, at least 8 months, at least 9 months, at least 10 months, at least 11 months, or at least 12 months. The antibody fragment may be administered throughout lifetime of the patient or intermittently throughout the lifetime of the patient, e.g., in response to a flare. In some embodiments, the antibody fragment is administered subcutaneously.

In some embodiments, the anti-C1q antibody inhibits the interaction between C1q and an autoantibody or between C1q and C1r, or between C1q and C1s and/or promotes clearance of C1q from circulation or a tissue. In some embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7, and/or a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, preferably the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11, preferably the heavy chain variable domain comprising an amino acid sequence selected from SEQ ID NO: 8 and 31-34. In some embodiments, the antibody fragment comprises heavy chain Fab fragment of SEQ ID NO: 39 and light chain Fab fragment of SEQ ID NO: 40.

In some embodiments, the inhibitor of the classical complement pathway is a C1r inhibitor, such as a small molecule, an antibody, an aptamer, an antisense nucleic acid or a gene editing agent, preferably an anti-C1r antibody. In some embodiments, the anti-C1r antibody inhibits the interaction between C1r and C1q or between C1r and C1s, or wherein the anti-C1r antibody inhibits the catalytic activity of C1r or inhibits the processing of pro-C1r to an active protease.

In some embodiments, the inhibitor of the classical complement pathway is a C1s inhibitor, such as a small molecule, an antibody, an aptamer, an antisense nucleic acid or a gene editing agent, preferably an anti-C1s antibody. In some embodiments, the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s. In some embodiments, the antibody is sutimlimab.

In some embodiments, the inhibitor of the classical complement pathway is an anti-C1 complex antibody, optionally wherein the anti-C1 complex antibody inhibits C1r or C1s activation or blocks their ability to act on C2 or C4. The anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s.

In some embodiments, the inhibitor of the classical complement pathway is a C2 inhibitor, such as a small molecule, an antibody, an aptamer, an antisense nucleic acid or a gene editing agent. The C2 inhibitor may be ARGX-117 (Argenx).

In some embodiments, the inhibitor of the classical complement pathway is a C3 inhibitor, such as a small molecule, an antibody, an aptamer, an antisense nucleic acid or a gene editing agent. The C3 inhibitor may be APL-9 (Apellis) or AMY-101 (Amyndas).

In some embodiments, the inhibitor of the classical complement pathway is a C4 inhibitor, such as a small molecule, an antibody, an aptamer, an antisense nucleic acid or a gene editing agent.

### DESCRIPTION OF THE FIGURES

FIG. 1 shows that Systemic Lupus Erythematosus (SLE) is driven by autoantibodies against numerous self antigens (including DNA).

FIG. 2 depicts that excess immune complex (IC) and C1q glomerular deposition induces local complement-mediated inflammation, disrupting Glomerular Basement Membrane (GBM) structure & function. PACA is Pathogenic Anti-C1q Antibodies.

FIG. 3 shows patient demographic.

FIG. 4 shows that higher circulating C4d/C4 ratio in patients with active Lupus Nephritis (LN) suggests complement may drive disease progression and severity.

FIG. 5 shows that LN patients with high C4d/C4 ratio exhibit coordinated classical complement activation.

FIG. 6 shows that high correlation among complement factors in LN pts suggests coordinated pathway activity.

FIG. 7 shows no correlation observed in Health controls.

FIGS. 8A-8B show that active LN patients with high C4d/C4 ratio are likely to have higher PACA1.

FIG. 9 shows that C4d/C4 is reduced post flare event by standard of care but effect is not durable.

FIG. 10 shows changes in urine protein/creatinine ratio (UPCR) and C4 in response to standard of care treatment.

FIG. 11 shows that clinical C4 measurements tracks UPCR trajectories.

FIG. 12 shows that rise in C4 and decreased C4d/C4 correlates with improved disease activity. SLEDAI (Systemic Lupus Erythematosus Disease Activity Index) is an objective metrics of overall disease activity, consistent of a list of organ manifestation in the most recent 10 days.

FIG. 13 shows that PACAs of the IgG1 and IgG3 isotypes provided the strongest degree of correlation with the UPCR, whereas there was no significant correlation with levels of IgG2 and IgG4 antibody against C1q.

FIG. 14 shows that PACA1 and PACA3 levels correlate with C4d and the C4d/C4 ratio.

FIG. 15 shows that deposition of C4d in kidney biopsies suggests involvement of classical complement pathway in LN.

FIG. 16 shows that high accuracy for this biomarker identifies SLE patients with LN.

FIG. 17 shows early study with animal model of lupus nephritis reports causal link between anti-C1q antibodies and kidney damage.

FIG. 18 shows that administration of PACA greatly exacerbates disease with large increase in complement deposition in glomerular subendothelial space.

FIG. 19 shows that PACA binding to substrate-bound C1q is not affected by soluble C1q.

FIG. 20 shows that anti-C1q IgG1 and IgG3, but not IgG4 levels correlate with serum C4d/C4.

FIGS. 21A-21P are box plots showing the complement factors in healthy controls (HC) and patients with active Lupus Nephritis. The boxes in the plot represent the 25th, 50th and 75th percentile of levels in the subjects.

FIG. 22 is a correlation matrix showing anti- or positive-correlation among complement factors in Lupus Nephritis patients and suggests coordinated pathway activity. The circle size indicates the correlation distance between features (row×column). The circle shading indicates the Spearman correlation distance ranging from anti-correlation, e.g., “−1” to positive correlation, e.g., “+1” as shown on the Y-axis. The significance of the correlation is represented by asterisks in the circles. The asterisks indicate the p-values for each correlation; namely, P-value <0.001 (***), P-value<0.01 (**); and P-value<0.05 (*).

FIGS. 23A-23F are graphs showing the treatment effect of FabA on the complement factors of patients with Lupus Nephritis.

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

Inhibition of C1q (assessed by free C1q in serum or plasma) with anti-C1q inhibitor, including but not limited to an anti-C1q Fab fragment, in Lupus Nephritis (LN) reduces downstream complement activation (e.g., complement factor C4, and its activation products, C2, and its activation products, C3, and its activation products, C5 and its activation products, etc.) and complement-mediated renal inflammation.

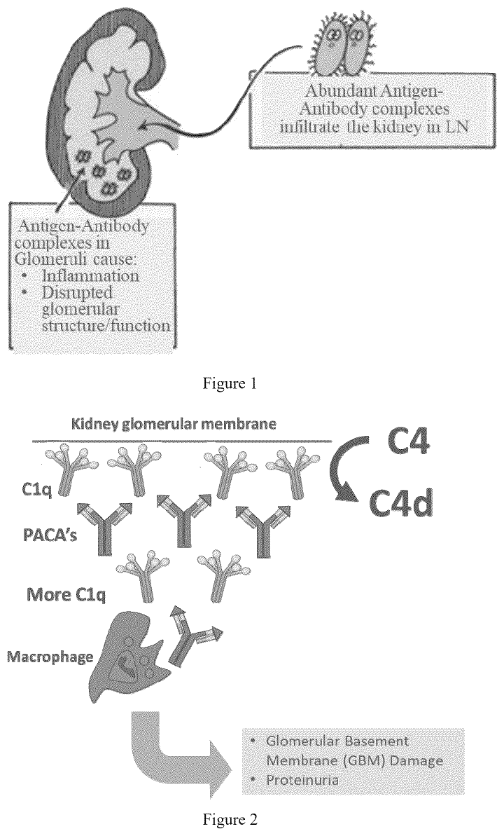

Lupus Nephritis may be an autoantibody-mediated disease with unique C1q/Classical complement cascade involvement. Systemic Lupus Erythematosus (SLE) is driven by autoantibodies against numerous self-antigens (including DNA) (FIG. 1). Normal clearance of immune complex (IC) is overwhelmed. Excess IC and C1q glomerular deposition induce local complement-mediated inflammation, disrupting Glomerular Basement Membrane (GBM) structure & function (FIG. 2). The data disclosed herein shows that high plasma C4d/C4 identifies lupus nephritis patients with disease mediated by activation of the classical complement pathway. PACA (Pathogenic Anti-C1q Antibodies) amplifies complement activation, perpetuating tissue damage. Complement activation via the classical pathway can be measured by the C4d/C4 ratio with high specificity.

### Unique Precision Medicine Strategy in Lupus Nephritis

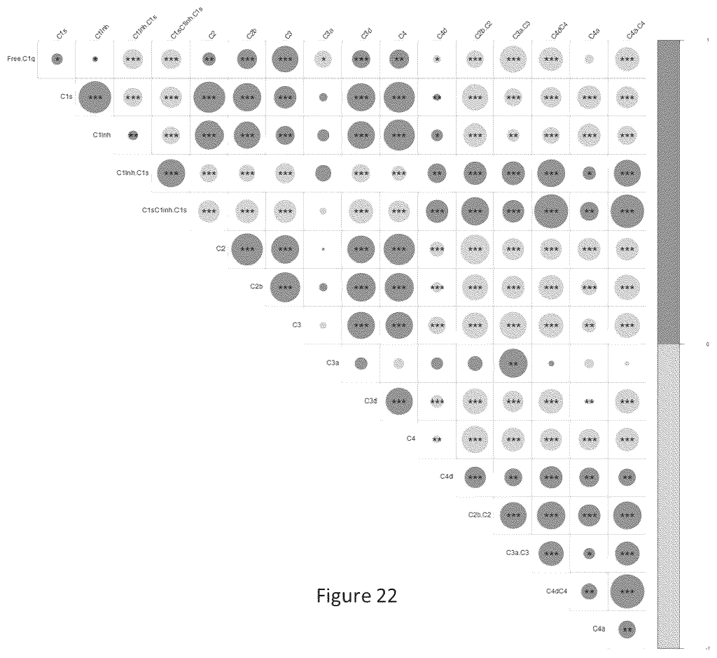

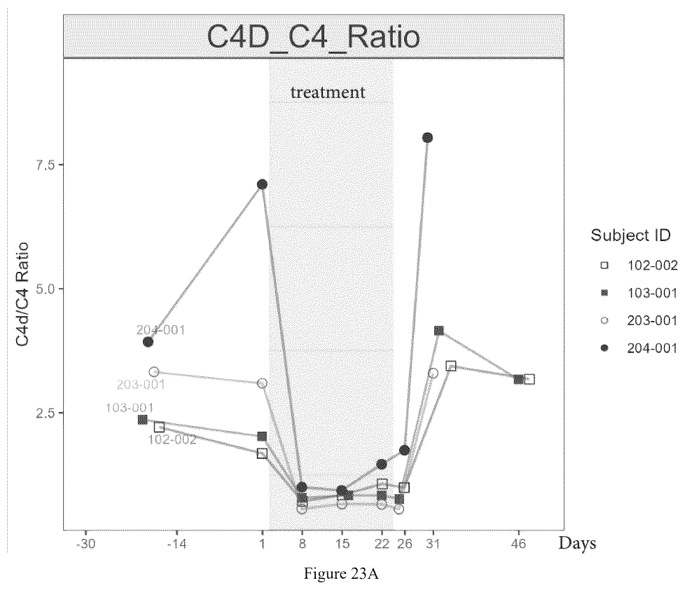

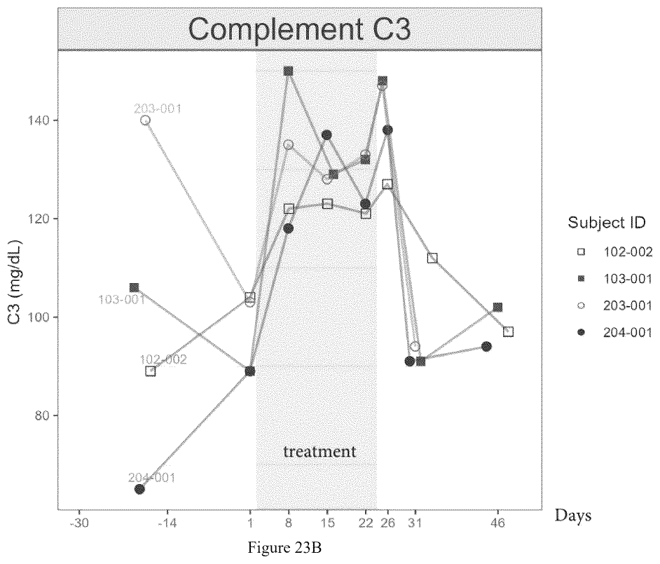

Data from Example 1 demonstrates that patients identified via the use of a model evaluating elevated C4d levels and/or elevated C4d/C4 ratios exhibit clinical improvement with anti-C1q antibody treatment. The data from Example 1 also shows strong association between high C4d/C4 ratio and presence of PACA and strong association between other complement factors in LN patients such as, for example, C4d/C4 and C4d have strong positive correlation, C2b and C2b/C2 have a strong anti-correlation (FIG. 22). The correlation is used to select for patients most likely to respond to therapy with a classical complement pathway, and more specifically with an anti-C1q therapy. Similarly, FIG. 22 shows a correlation matrix showing anti- or positive-correlation among the other complement factors (such as C4x level; C4x/C4 ratio; C4 level; C1sC1 inhibitor level; C1sC1 inhibitor/C1s ratio; C1s level; C2b level; C2b/C2 ratio; C2 level; Pathogenic Anti-C1q Antibody 1 (PACA1) and/or Pathogenic Anti-C1q Antibody 3 (PACA3) level) in Lupus Nephritis patients and suggests coordinated pathway activity.

The present disclosure is generally directed to methods of treating lupus nephritis in a subject in need thereof. The method comprises determining that the subject has at least one of the following characteristics: an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. C4x may be selected from the group consisting of C4a and C4d. The method further comprises administering to the subject an inhibitor of the classical complement pathway, e.g., if the subject has an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. A therapeutically effective amount of the inhibitor may be administered.

All sequences mentioned in the present disclosure are incorporated by reference from U.S. Pat. No. 10,316,081, U.S. patent application Ser. No. 14/890,811, U.S. Pat. Nos. 8,877,197, 9,708,394, U.S. patent application Ser. No. 15/360,549, U.S. Pat. Nos. 9,562,106, 10,450,382, 10,457,745, International Patent Application No. PCT/US2018/022462 each of which is hereby incorporated by reference for the antibodies and related compositions disclosed.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

As used herein “reference level” relates to a predetermined criteria used as a reference for evaluating the values or data obtained from a sample obtained from an individual. The reference level can be an absolute value; a relative value; a value that has an upper or a lower limit; a range of values; an average value; a median value; a mean value; or a value as compared to a particular control or baseline value. A reference level can be based on an individual sample value, such as for example, a value obtained from a sample from the subject being tested, but at an earlier point in time. The reference level can be based on a large number of samples, such as from a population of subjects of similar chronological age, gender, disease state, or otherwise matched group, or based on a pool of samples including or excluding the sample to be tested. A reference level can also be determined from a representative number of samples (e.g., plasma) derived from different individuals afflicted with lupus nephritis. A reference level can also be determined from biological samples from non-lupus nephritis afflicted individuals (i.e., normal or healthy subjects of a similar age). These biological samples from a lupus nephritis afflicted or non-lupus nephritis afflicted individual may comprise for example, tissue biopsies, blood, plasma, serum, fecal samples, urine, cerebral spinal fluid, pap smears, or semen. A representative sample can include measurements from at least 10, 20, 30, 40, 50, 100, 200, 300, 400, 500, 600, 700, 800, 900, 1,000 or more individuals.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit the desired biological activity, and antibody derivatives.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The y and a classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

The term “agent” as used herein describes any molecule, e.g. protein or pharmaceutical, with the capability of modulating synapse loss, particularly through the complement pathway. Candidate agents also include genetic elements, e.g., anti-sense and RNAi molecules to inhibit C1q expression, and constructs encoding complement inhibitors, e.g., CD59, and the like. Candidate agents encompass numerous chemical classes, though typically they are organic molecules, including small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally, a plurality of assay mixtures are run in parallel with different agent concentrations to obtain a differential response to the various concentrations. Typically one of these concentrations serves as a negative control, i.e., at zero concentration or below the level of detection.

The “variable region” or “variable domain” of an antibody refers to the amino-terminal domains of the heavy or light chain of the antibody. The variable domains of the heavy chain and light chain may be referred to as “Vn” and “Vi.”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous since they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Nat'l Acad. Sci. USA 101 (34): 12467-472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

“Full-length antibodies” are usually heterotetrameric glycoproteins of about 150,000 daltons, comprising two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The terms “full-length antibody,” “intact antibody” and “whole antibody” are used interchangeably to refer to an antibody in its substantially intact form, as opposed to an antibody fragment or antibody derivative. Specifically, whole antibodies include those with heavy and light chains including an Fe region. The constant domains may be native sequence constant domains (e.g., human native sequence constant domains) or amino acid sequence variants thereof. In some cases, the intact antibody may have one or more effector functions.

An “antibody fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8 (10): 1057-1062 (1995)). Additional examples of antibody fragments include antibody derivatives such as single-chain antibody molecules, single-arm antibodies, antibodies with a single antigen-binding arm, monovalent antibodies and multispecific antibodies formed from antibody fragments

The term “single-arm antibody” herein is used to cover an antibody that comprises a single antigen-binding arm. The single-arm antibody may comprise an antigen-binding arm and an Fc region, wherein the single antigen-binding arm comprises a light chain variable domain and a heavy chain variable domain; and the Fc region comprises a complex of a first and a second Fc polypeptide. In some embodiments, one but not both of the Fc polypeptides is an N-terminally truncated heavy chain. In some embodiments, the antibody may be a bivalent antibody—where one arm binds C1q and the other binds a different antigen. Depending on the other antigen, such an antibody would not crosslink and activate C1q.

“An antibody with a single antigen-binding arm” as used herein means an antibody comprising a single antigen-binding arm and an Fc region, wherein the antigen-binding arm comprises a light chain variable domain and a heavy chain variable domain. In some embodiments, the antibody further comprises an inactive antigen-binding arm, which is incapable of binding to the antigen, or comprises an arm that binds to a different antigen. In some embodiments, the Fc region comprises a complex of a first and a second Fc polypeptide.

An “antibody derivative” is any construct that comprises the antigen binding region of an antibody. Examples of antibody derivatives include single-chain antibody molecules, single arm antibodies, antibodies with a single antigen-binding arm, monovalent antibodies and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the Cal domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fe regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fe region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FeyRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (See, e.g., M. Daëron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. FcRs can also increase the serum half-life of antibodies.

Binding to FoRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9 (2): 6591-6604 (2001).

“Fv”′ is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, NJ, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabaf” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169: 147-155 (1995); Yelton et al., J. Immunol. 155: 1994-2004 (1995); Jackson et al., J. Immunol. 154 (7): 3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains),

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

The term “inhibitor” refers to a compound having the ability to inhibit a biological function of a target biomolecule, for example, an mRNA or a protein, whether by decreasing the activity or expression of the target biomolecule. An inhibitor may be an antibody, a small molecule, or a nucleic acid molecule. The term “antagonist” refers to a compound that binds to a receptor, and blocks or dampens the receptor's biological response. The term “inhibitor” may also refer to an “antagonist.”

Antibody “effector functions” refer to those biological activities attributable to the Fc region (a native sequence Fe region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-C1s antibody binds specifically to an epitope within a complement C1s protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “Pathogenic Anti-C1q Antibody” or “Pathogenic Anti-C1q Antibodies” or “PACA” or “PACAs” as used herein refer to endogenous autoantibodies against C1q that selectively recognize a neoepitope in the collagen tail of C1q when it is bound to a substrate and thereby recruit additional C1q. PACA is made up of four IgG subclasses, IgG1, IgG2, IgG3, and IgG4, which are referred to as PACA1, PACA2, PACA3 and PACA4, respectively. PACAs recognize the collagen tail of C1q when it is bound to substrate and recruit additional C1q via their Fc region, thereby enhancing C1q activity. Without wishing to be bound by theory, PACAs can amplify glomerular injury when bound within the glomerulus to C1q that has been already brought to that site by other types of glomerular-reactive autoantibodies.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### C1q Inhibitors

The inhibitor of the classical complement pathway may be a C1q inhibitor, such as a small molecule, an antibody, an aptamer, an antisense nucleic acid or a gene editing agent.

The anti-C1q antibodies disclosed herein are potent inhibitors of C1q.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂).

Suitable inhibitors include an antibody that binds complement factor C1q and/or C1q in the C1 complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr₂s₂).

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:
C1q, chain A (Homo sapiens), Accession No. Protein
Data Base: NP_057075.1; GenBank No.: NM 015991:
>gi|7705753|ref|NP_057075.1|complement C1q
subcomponent subunit A precursor [Homo sapiens]

(SEQ ID NO: 1)
MEGPRGWLVLCVLAISLASMVTEDLCRAPDGKKGEAGRPGRRGRPGLK
GEQGEPGAPGIRTGIQGLKGDQGEPGPSGNPGKVGYPGPSGPLGARGI
PGIKGTKGSPGNIKDQPRPAFSAIRRNPPMGGNVVIFDTVITNQEEPY
QNHSGRFVCTVPGYYYFTFQVLSQWEICLSIVSSSRGQVRRSLGFCDT
TNKGLFQVVSGGMVLQLQQGDQVWVEKDPKKGHIYQGSEADSVFSGFL
IFPSA.

C1q, chain B (Homo sapiens), Accession No. Protein
Data Base: NP_000482.3; GenBank No.: NM. 000491.3:
>gi|87298828|ref|NP_000482.3|complement C1q
subcomponent subunit B precursor [Homo sapiens]

(SEQ ID NO: 52)
MMMKIPWGSIPVLMLLLLLGLIDISQAQLSCTGPPAIPGIPGIPGTPG
PDGQPGTPGIKGEKGLPGLAGDHGEFGEKGDPGIPGNPGKVGPKGPMG
PKGGPGAPGAPGPKGESGDYKATQKIAFSATRTINVPLRRDQTIREDH
SSRGNLCVNLMRGRERAQKVVTFCDYAYNTFQVTTGGMVLKLEQGENV
FVITNMNNNYEPRSGKFTCKVPGLYYFTYHALQATDKNSLLGMEGANS
IFSGFLLFPDMEA.

C1q, chain C (Homo sapiens), Accession No. Protein
Data Base: NP_001107573.1; GenBank No.:
NM 001114101.1:
>gi|166235903|ref|NP_001107573.1|complement C1q
subcomponent subunit C precursor [Homo sapiens]

(SEQ ID NO: 53)
MDVGPSSLPHLGLKLLLLLLLLPLRGQANTGCYGIPGMPGLPGAPGKD
GYDGLPGPKGEPGIPAIPGIRGPKGQKGEPGLPGHPGKNGPMGPPGMP
GVPGPMGIPGEPGEEGRYKQKFQSVFTVTRQTHQPPAPNSLIRFNAVL
TNPQGDYDTSTGKFTCKVPGLYYFVYHASHTANLCVLLYRSGVKVVTF
CGHTSKTNQVNSGGVLLRLQVGEEVWLAVNDYYDMVGIQGSDSVFSGF
LLFPD.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, or a chimeric antibody.

Other anti-C1q antibodies suitable for binding to C1q protein are well-known in the art and include, for example, antibodies Cat #: AF2379, AF1696, MAB1696, and MAB23791 (R&D System), NBP1-87492, NB100-64420, H00000712-B01P, H00000712-D01P, and H00000712-D01 (Novus Biologicals), MA1-83963, MA1-40311, PA5-14208, PA5-29586, and PA1-36177 (ThermoFisher Scientific), ab71940, ab11861, ab4223, ab72355, ab182451, ab46191, ab227072, ab182940, ab216979, and ab235454 (abcam), etc. Moreover, multiple siRNA, shRNA, CRISPR constructs for reducing C1q expression can be found in the commercial product lists of the above-referenced companies, such as SiRNA product #sc-43651, sc-44962, sc-105153, sc-141842, ShRNA product #sc-43651-SH, sc-43651-V, sc-44962-SH, sc-44962-V, sc-105153-SH, sc-105153-V, sc-141842-SH, sc-141842-V, CRISPR product #sc-419385, sc-419385-HDR, sc-419385-NIC, sc-419385-NIC-2, sc-402156, sc-402156-KO-2, sc-404309, sc-404309-HDR, sc-404309-NIC, sc-404309-NIC-2, sc-419386, sc-419386-HDR, sc-419386-NIC, sc-419386-NIC-2 (Santa Cruz Biotechnology, etc).

Light Chain and Heavy Chain Hypervariable Region Sequences and Variable Domain Sequences of Antibody M1 (Mab1), Incorporated by Reference from U.S. Pat. No. 9,708,394)

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWYQEKPGKTNKLLI
YSGSTLQSGIPSRFSGSGSGTDFTLTISSLEPEDFAMYYCQQHNEYPL
TFGAGTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the M1 light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHWVKQRPGQGLEWI
GVIHPNSGSINYNEKFESKATLTVDKSSSTAYMQLSSLTSEDSAVYYC
AGERDSTEVLPMDYWGQGTSVTVSS.

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIHPNSGSINYNEKFES (SEQ ID NO:10), and the HVR-H3 of the M1 heavy chain variable domain has the sequence

(SEQ ID NO: 11)
ERDSTEVLPMDY.

The nucleic acid sequence encoding the light chain variable domain was determined to be:

(SEQ ID NO: 12)
GATGTCCAGATAACCCAGTCTCCATCTTATCTTGCTGCATCTCCTGGA
GAAACCATTACTATTAATTGCAGGGCAAGTAAGAGCATTAACAAATAT
TTAGCCTGGTATCAAGAGAAACCTGGGAAAACTAATAAGCTTCTTATC
TACTCTGGATCCACTTTGCAATCTGGAATTCCATCAAGGTTCAGTGGC
AGTGGATCTGGTACAGATTTCACTCTCACCATCAGTAGCCTGGAGCCT
GAAGATTTTGCAATGTATTACTGTCAACAACATAATGAATACCCGCTC
ACGTTCGGTGCTGGGACCAAGCTGGAGCTGAAA.

The nucleic acid sequence encoding the heavy chain variable domain was determined to be:

(SEQ ID NO: 13)
CAGGTCCAACTGCAGCAGCCTGGGGCTGAGCTGGTAAAGCCTGGGGCT
TCAGTGAAGTTGTCCTGCAAGTCTTCTGGCTACCATTTCACCAGCTAC
TGGATGCACTGGGTGAAGCAGAGGCCTGGACAAGGCCTTGAGTGGATT
GGAGTGATTCATCCTAATAGTGGTAGTATTAACTACAATGAGAAGTTC
GAGAGCAAGGCCACACTGACTGTAGACAAATCCTCCAGCACAGCCTAC
ATGCAACTCAGCAGCCTGACATCTGAGGACTCGGCGGTCTATTATTGT
GCAGGAGAGAGAGATTCTACGGAGGTTCTCCCTATGGACTACTGGGGT
CAAGGAACCTCAGTCACCGTCTCCTCA.

The Mab1-Fab is the Fab of the Mab1 (M1) antibody.

Mab3 is a murine anti-C1q antibody that is derived from Mab1 antibody and optimized for murine experiments.

### Deposit of Material

The following materials have been deposited according to the Budapest Treaty in the American Type Culture Collection, ATCC Patent Depository, 10801 University Blvd., Manassas, Va. 20110-2209, USA (ATCC):

### Deposit ATCC

Isotype Date Accession No. Sample ID
Mouse hybridoma C1qM1 IgG1, Jon. 6, PTA-120399
7788-1 (M) 051613 producing kappa 2013
anti-C1q antibody M1

The hybridoma cell line producing the M1 antibody (mouse hybridoma C1qM1.7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

Disclosed herein are methods of administering an anti-C1q antibody comprising a light chain variable domain and a heavy chain variable domain. The antibody may bind to at least human C1q, mouse C1q, or rat C1q. The antibody may be a humanized antibody, a chimeric antibody, or a human antibody. The antibody may be a monoclonal antibody, an antibody fragment thereof, and/or an antibody derivative thereof. The light chain variable domain comprises the HVR-L1, HVR-L2, and HVR-L3 of the monoclonal antibody M1 produced by a hybridoma cell line deposited with Accession Number PTA-120399. The heavy chain variable domain comprises the HVR-H1, HVR-H2, and HVR-H3 of the monoclonal antibody M1 produced by a hybridoma cell line deposited with ATCC Accession Number PTA-120399.

In some embodiments, the amino acid sequence of the light chain variable domain and heavy chain variable domain comprise one or more of SEQ ID NO:5 of HVR-L1, SEQ ID NO: 6 of HVR-L2, SEQ ID NO:7 of HVR-L3, SEQ ID NO:9 of HVR-H1, SEQ ID NO:10 of HVR-H2, and SEQ ID NO: 11 of HVR-H3.

The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO: 11).

Disclosed herein are methods of administering an anti-C1q antibody, which inhibits the interaction between C1q and an autoantibody. In preferred embodiments, the anti-C1q antibody causes clearance of C1q from the circulation or tissue.

In some embodiments, the anti-C1q antibody of this disclosure inhibits the interaction between C1q and C1s. In some embodiments, the anti-C1q antibody inhibits the interaction between C1q and C1r. In some embodiments, the anti-C1q antibody inhibits the interaction between C1q and C1s and between C1q and C1r. In some embodiments, the anti-C1q antibody inhibits the interaction between C1q and another antibody, such as an autoantibody. In preferred embodiments, the anti-C1q antibody causes clearance of C1q from the circulation or tissue. In some embodiments, the anti-C1q antibody inhibits the respective interactions, at a stoichiometry of less than 2.5:1; 2.0:1; 1.5:1; or 1.0:1. In some embodiments, the C1q antibody inhibits an interaction, such as the C1q-C1s interaction, at approximately equimolar concentrations of C1q and the anti-C1q antibody. In other embodiments, the anti-C1q antibody binds to C1q with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1q antibody binds C1q with a binding stoichiometry that ranges from 20:1 to 1.0:1 or less than 1.0:1. In certain embodiments, the anti-C1q antibody binds C1q with a binding stoichiometry that ranges from 6:1 to 1.0:1 or less than 1.0:1. In certain embodiments, the anti-C1q antibody binds C1q with a binding stoichiometry that ranges from 2.5:1 to 1.0:1 or less than 1.0:1. In some embodiments, the anti-C1q antibody inhibits the interaction between C1q and C1r, or between C1q and C1s, or between C1q and both C1r and C1s. In some embodiments, the anti-C1q antibody inhibits the interaction between C1q and C1r, between C1q and C1s, and/or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain. In some embodiments, the anti-C1q antibody binds to the C1q A-chain, the C1q B-chain and/or the C1q C-chain. In some embodiments, the anti-C1q antibody binds to the globular domain of the C1q A-chain, B-chain, and/or C-chain. In other embodiments, the anti-C1q antibody binds to the collagen-like domain of the C1q A-chain, the C1q B-chain, and/or the C1q C-chain.

Where antibodies of this disclosure inhibit the interaction between two or more complement factors, such as the interaction of C1q and C1s, or the interaction between C1q and C1r, the interaction occurring in the presence of the antibody may be reduced by at least 10%, at least 20%, at least 30%, at least 40%, at least 50%, at least 60%, at least 70%, at least 80%, at least 90%, at least 95%, or at least 99% relative to a control wherein the antibodies of this disclosure are absent. In certain embodiments, the interaction occurring in the presence of the antibody is reduced by an amount that ranges from at least 30% to at least 99% relative to a control wherein the antibodies of this disclosure are absent.

In some embodiments, the antibodies of this disclosure inhibit C2 or C4-cleavage by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring C2 or C4-cleavage are well known in the art. The EC₅₀ values for antibodies of this disclosure with respect C2 or C4-cleavage may be less than 3 μg/ml; 2.5 μg/ml; 2.0 ng/ml; 1.5 μg/ml; 1.0 g/ml; 0.5 μg/ml; 0.25 μg/ml; 0.1 μg/ml; 0.05 μg/ml. In some embodiments, the antibodies of this disclosure inhibit C2 or C4-cleavage at approximately equimolar concentrations of C1q and the respective anti-C1q antibody.

In some embodiments, the antibodies of this disclosure inhibit autoantibody-dependent and complement-dependent cytotoxicity (CDC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. The EC₅₀ values for antibodies of this disclosure with respect to inhibition of autoantibody-dependent and complement-dependent cytotoxicity may be less than 3 ng/ml; 2.5 μg/ml; 2.0 μg/ml; 1.5 μg/ml; 1.0 ng/ml; 0.5 μg/ml; 0.25 ng/ml; 0.1 μg/ml; 0.05 μg/ml.

In some embodiments, the antibodies of this disclosure inhibit complement-dependent cell-mediated cytotoxicity (CDCC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring CDCC are well known in the art. The BC50 values for antibodies of this disclosure with respect CDCC inhibition may be 1 less than 3 μg/ml; 2.5 μg/ml; 2.0 μg/ml; 1.5 μg/ml; 1.0 ng/ml; 0.5 μg/ml; 0.25 μg/ml; 0.1 μg/ml; 0.05 μg/ml. In some embodiments, the antibodies of this disclosure inhibit CDCC but not antibody-dependent cellular cytotoxicity (ADCC).

Humanized Anti-Complement C1q Antibodies (Incorporated by Reference from U.S. Pat. No. 10,316,081)

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat C1q.

In some embodiments, the human heavy chain constant region is a human IgG4 heavy chain constant region comprising the amino acid sequence of SEQ ID NO:47, or with at least 70%, at least about 75%, at least about 80%, at least about 85%, at least about 90% homology to SEQ ID NO: 47. The human IgG4 heavy chain constant region may comprise an Fc region with one or more modifications and/or amino acid substitutions according to Kabat numbering. In such cases, the Fc region comprises a leucine to glutamate amino acid substitution at position 248 (corresponding to L115E mutation in IgG4), wherein such a substitution inhibits the Fc region from interacting with an Fc receptor. In some embodiments, the Fc region comprises a serine to proline amino acid substitution at position 241 (corresponding to S108P in IgG4), wherein such a substitution prevents arm switching in the antibody.

The amino acid sequence of human IgG4 (S241P L248E; that is corresponding to S108P and L11SE in SEQ ID NO: 47) heavy chain constant domain is:

(SEQ ID NO: 47)
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPPCPAPEFEGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38.

The amino acid sequence of heavy chain variable domain variant 1 (VH1) is:
(SEQ ID NO: 31)
QVQLVQSGAELKKPGASVKVSCKSSGYHFTSYWMHWVKQAPGQGLEWIGVIHPN
SGSINYNEKFESKATITVDKSTSTAYMQLSSLTSEDSAVYYCAGERDSTEVLPMDY
WGQGTSVTVSS.
The hyper variable regions (HVRs) of VH1 are depicted in bolded and underlined text.
The amino acid sequence of heavy chain variable domain variant 2 (VH2) is:
(SEQ ID NO: 32)
QVQLVQSGAELKKPGASVKVSCKSSGYHIFTSYWMHWVKQAPGQGLEWIGVIHPN
SGSINYNEKFESRATITVDKSTSTAYMELSSLRSEDTAVYYCAGERDSTEVLPMDY
WGQGTTVTVSS.
The hyper variable regions (HVRs) of VH2 are depicted in bolded and underlined text.
The amino acid sequence of heavy chain variable domain variant 3 (VH3) is:
(SEQ ID NO: 33)
QVQLVQSGAELKKPGASVKVSCKSSGYHFTSYWMHWVKQAPGQGLEWIGVIHPN
SGSINYNEKFESRVTITVDKSTSTAYMELSSLRSEDTAVYYCAGERDSTEVLPMDY
WGQGTTVTVSS.
The hyper variable regions (HVRs) of VH3 are depicted in bolded and underlined text.
The amino acid sequence of heavy chain variable domain variant 4 (VH4) is:
(SEQ ID NO: 34)
QVQLVQSGAELKKPGASVKVSCKSSGYHFTSYWMHWVRQAPGQGLEWIGVIHPN
SGSINYNEKFESRVTITVDKSTSTAYMELSSLRSEDTAVYYCAGERDSTEVLPMDY
WGQGTTVTVSS.
The hyper variable regions (HVRs) of VH4 are depicted in bolded and underlined text.
The amino acid sequence of kappa light chain variable domain variant 1 (Vκ1) is:
(SEQ ID NO: 35)
DVQITQSPSYLAASLGERATINCRASKSINKYLAWYQQKPGKTNKLLIYSGSTLQSGI
PARFSGSGSGTDFTLTISSLEPEDFAMYYCQQHNEYPLTFGQGTKLEIK.
The hyper variable regions (HVRs) of Vx1 are depicted in bolded and underlined text.
The amino acid sequence of kappa light chain variable domain variant 2 (Vκ2) is:
(SEQ ID NO: 36)
DVQITQSPSSLSASLGERATINCRASKSINKYLAWYQQKPGKANKLLIYSGSTLQSGI
PARFSGSGSGTDFTLTISSLEPEDFAMYYCQQHNEYPLTFGQGTKLEIK.
The hyper variable regions (HVRs) of Vx2 are depicted in bolded and underlined text.
The amino acid sequence of kappa light chain variable domain variant 3 (Vκ3) is:
(SEQ ID NO: 37)
DVQITQSPSSLSASLGERATINCRASKSINKYLAWYQQKPGKAPKLLIYSGSTLQSGI
PARFSGSGSGTDFTLTISSLEPEDFAMYYCQQHNEYPLTFGQGTKLEIK.
The hyper variable regions (HVRs) of Vx3 are depicted in bolded and underlined text.
The amino acid sequence of kappa light chain variable domain variant 4 (Vκ4) is:
(SEQ ID NO: 38)
DIQLTQSPSSLSASLGERATINCRASKSINKYLAWYQQKPGKAPKLLIYSGSTLQSGIP
ARFSGSGSGTDFTLTISSLEPEDFAMYYCQQHNEYPLTFGQGTKLEIK.
The hyper variable regions (HVRs) of Vx4 are depicted in bolded and underlined text.

The antibody may comprise a light chain variable domain amino acid sequence that is at least 85%, 90%, or 95% identical to SEQ ID NO:35-38 while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:31-34 while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO: 11).

In some embodiments, the antibody comprises a light chain variable domain amino acid sequence of SEQ ID NO: 35 and a heavy chain variable domain amino acid sequence of SEQ ID NO: 31. In some embodiments, the antibody comprises a light chain variable domain amino acid sequence of SEQ ID NO: 36 and a heavy chain variable domain amino acid sequence of SEQ ID NO: 32. In some embodiments, the antibody comprises a light chain variable domain amino acid sequence of SEQ ID NO: 37 and a heavy chain variable domain amino acid sequence of SEQ ID NO: 33. In some embodiments, the antibody comprises a light chain variable domain amino acid sequence of SEQ ID NO: 38 and a heavy chain variable domain amino acid sequence of SEQ ID NO: 34.

The full-length antibody Mab2 comprises the heavy chain variable domain variant 3 (VH3) (SEQ ID NO: 33) and the kappa light chain variable domain variant 3 (Vκ3) (SEQ Id NO: 37). The Mab2-Fab is the Fab of the Mab2 antibody.

The antibody may comprise a heavy chain and a light chain, wherein the heavy chain comprises the amino acid sequence of SEQ ID NO: 14; and the light chain comprises the amino acid sequence of SEQ ID NO: 40.

The amino acid sequence of the heavy chain is:
(SEQ ID NO: 14)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFEGGPSVFLFP
PKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK. The hyper variable regions (HVRs) of
VH3 are depicted in bolded and underlined text.
The amino acid sequence of the light chain is:
(SEQ ID NO: 40)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The complementarity determining regions (CDRs) of SEQ ID NO:40 are depicted in bolded and underlined text.

In some embodiments, humanized anti-C1q antibodies of the present disclosure include a heavy chain variable region that contains a Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or lgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a position corresponding to position 248 inhibits the Fc region from interacting with an Fc receptor. In some embodiments, the amino acid substitution at position 248 or a position corresponding to position 248 is a leucine to glutamate amino acid substitution. In some embodiments, the amino acid substitution at position 241 or a position corresponding to position 241 prevents arm switching in the antibody. In some embodiments, the amino acid substitution at position 241 or a position corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

### Anti-C1q Fab Fragment (e.g., FabA)

All anti-C1q antibody Fab fragment sequences are incorporated by reference from U.S. Pat. No. 10,723,788, which is hereby incorporated by reference for the antibodies and related compositions that it discloses.

In certain embodiments, the present disclosure provides an anti-C1q antibody Fab fragment that binds to a C1q protein comprising a heavy (VH/CH1) and light chain (VL/CL), wherein the anti-C1q antibody Fab fragment has six complementarity determining regions (CDRs), three each from VL and VH (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence:

(SEQ ID NO: 39)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHT

The complementarity determining regions (CDRs) of SEQ ID NO:39 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKPGKAPKL
LIYSGSTLQSGIPARFSGSGSGTDFTLTISSLEPEDFAMYYCQQHN
EYPLTFGQGTKLEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC

The complementarity determining regions (CDRs) of SEQ ID NO:40 are depicted in bolded and underlined text.

The FabA is an anti-C1q antibody Fab fragment comprising the heavy chain domain comprising SEQ ID NO: 39 and the light chain domain comprising SEQ ID NO: 40.

The Mab1-Fab is the Fab of the Mab1 (M1) antibody.

The Mab2-Fab is the Fab of the Mab2 antibody.

The Mab3-Fab is the Fab of the Mab3 antibody.

### Anti-C1q Single-Arm Antibody

All anti-C1q single arm antibody sequences disclosed in U.S. Pat. App. No. 63/288,883 and U.S. Pat. App. No. 63/288,885, which are hereby incorporated by reference for the antibodies and related compositions they disclose.

In certain aspects, the present disclosure provides an antibody that binds to a protein in the complement cascade, such as a C1q protein. The antibody that binds to C1q comprises a single C1q antigen-binding arm and an Fc region. The single C1q antigen-binding arm may comprise a light chain variable domain and a heavy chain variable domain. The Fc region may comprise a complex of a first and a second Fc polypeptide. The Fc region may comprise a Fcγ receptor binding site mutation. The antibody may be of the IgG4 class. In some embodiments, one but not both of the Fc polypeptide is an N-terminally truncated heavy chain. In some embodiments, the Fcγ receptor is FcγRI, FcγRII, or FcγRIII, preferably FcγRI. The Fcγ receptor binding site mutation may comprise a IgG4 L115E mutation.

(SEQ ID NO: 40)
DVQITQSPSSLSASLGERATINCRASKSINKYLAWYQQKPGKAPKL
LIYSGSTLQSGIPARFSGSGSGTDFTLTISSLEPEDFAMYYCQQHN
EYPLTFGQGTKLEIKRTVAAPSVEIFPPSDEQLKSGTASVVCLLNN
FYPREAKVQWKVDNALQSGNSQESVTEQDSKDSTYSLSSTLTLSKA
DYEKHKVYACEVTHQGLSSPVTKSENRGEC

The complementarity determining regions (CDRs) of SEQ ID NO: 40 are depicted in bolded and underlined text. In some embodiments, the HVR-L1 of the light chain variable domain has the sequence RASKSINKYLA (SEQ ID NO:5), the HVR-L2 of the light chain variable domain has the sequence SGSTLQS (SEQ ID NO:6), and the HVR-L3 of the light chain variable domain has the sequence QQHNEYPLT (SEQ ID NO:7).

The light chain of the single-arm antibody may comprise the following light chain variable domain amino acid sequence:

(SEQ ID NO: 4)
DVQITQSPSYLAASPGETITINCRASKSINKYLAWYQEKPGKTNKL
LIYSGSTLQSGIPSRFSGSGSGTDFTLTISSLEPEDFAMYYCQQHN
EYPLTFGAGTKLELK.

The single-arm antibody may comprise a light chain variable domain amino acid sequence that is at least 85%, 90%, or 95% identical to SEQ ID NO: 10, preferably while retaining the HVR-L1 RASKSINKYLA (SEQ ID NO: 5), the HVR-L2 SGSTLQS (SEQ ID NO: 6), and the HVR-L3 QQHNEYPLT (SEQ ID NO: 7).

The single-arm antibody may comprise the following amino acid sequence of kappa light chain variable domain variant 1 (Vκ1):

(SEQ ID NO: 35)
DVQITQSPSYLAASLGERATINCRASKSINKYLAWYQQKPGKTNKLLIYS
GSTLQSGIPARFSGSGSGTDFTLTISSLEPEDFAMYYCQQHNEYPLTFGQ
GTKLEIK.

The hyper variable regions (HVRs) of Vκ1 are depicted in bolded and underlined text.

The single-arm antibody may comprise the following amino acid sequence of kappa light chain variable domain variant 2 (Vκ2):

(SEQ ID NO: 36)
DVQITQSPSSLSASLGERATINCRASKSINKYLAWYQQKPGKANKLLIYS
GSTLQSGIPARFSGSGSGTDFTLTISSLEPEDFAMYYCQQHNEYPLTFGQ
GTKLEIK.

The hyper variable regions (HVRs) of Vκ2 are depicted in bolded and underlined text.

The single-arm antibody may comprise the following amino acid sequence of kappa light chain variable domain variant 3 (Vκ3):

(SEQ ID NO: 37)
DVQITQSPSSLSASLGERATINCRASKSINKYLAWYQQKPGKAPKLLIYS
GSTLQSGIPARFSGSGSGTDFTLTISSLEPEDFAMYYCQQHNEYPLTFGQ
GTKLEIK.

The hyper variable regions (HVRs) of Vκ3 are depicted in bolded and underlined text.

The single-arm antibody may comprise the following amino acid sequence of kappa light chain variable domain variant 4 (Vκ4):

(SEQ ID NO: 38)
DIQLTQSPSSLSASLGERATINCRASKSINKYLAWYQQKPGKAPKLLIYS
GSTLQSGIPARFSGSGSGTDFTLTISSLEPEDFAMYYCQQHNEYPLTFGQ
GTKLEIK.

The hyper variable regions (HVRs) of Vκ4 are depicted in bolded and underlined text.

The single-arm antibody may comprise a light chain variable domain amino acid sequence that is at least 85%, 90%, or 95% identical to SEQ ID NO: 11-14 while retaining the HVR-L1 RASKSINKYLA (SEQ ID NO: 5), the HVR-L2 SGSTLQS (SEQ ID NO: 6), and the HVR-L3 QQHNEYPLT (SEQ ID NO: 7).

The antibody may be of the IgG4 class. The sequence of IgG4 heavy chain is

(SEQ ID NO: 41)

| | |
|---|---|
| 1 | ASTKGPSVFP LAPCSRSTSE STAALGCLVK |
| | DYFPEPVTVS WNSGALTSGV HTFPAVLQSS |
| 61 | GLYSLSSVVT VPSSSLGTKT YTCNVDHKPS |
| | NTKVDKRVES KYGPPCPSCP APEFLGGPSV |
| 121 | FLFPPKPKDT LMISRTPEVT CVVVDVSQED |
| | PEVQFNWYVD GVEVHNAKTK PREEQFNSTY |
| 181 | RVVSVLTVLH QDWINGKEYK CKVSNKGLPS |
| | SIEKTISKAK GQPREPQVYT LPPSQEEMTK |
| 241 | NQVSLTCLVK GFYPSDIAVE WESNGQPENN |
| | YKTTPPVLDS DGSFFLYSRL TVDKSRWQEG |
| 301 | NVFSCSVMHE ALHNHYTQKS LSLSLGK |

The domains of IgG4 are as follow: CH1: 1-98, Hinge: 99-110, CH2: 111-220, and CH3: 221-327. IgG4 may comprise mutations. For example, S108P mutation (for IgG4 arm swapping), L115E mutation (for FcR binding), T246W mutation (for knob in hole mutation), T246S mutation (for knob in hole mutation), L248A mutation (for knob in hole mutation), Y187V mutation (for knob in hole mutation), and/or N187A (aglycosylated for FcR binding), N187Q (aglycosylated for FcR binding), or N187G (aglycosylated for FcR binding).

One heavy chain of the single-arm antibody (the heavy chain 1 domain) of the single-arm antibody may comprise the following amino acid sequence (SEQ ID NO:2):

(SEQ ID NO: 2)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLWCLVKGFYPSDIAVEWESNGQPENNYKTTPPVLDSDGSF
FLYSRLTVDKSRWQEGNVFSCSVMHEALHNHYTQKSLSLSLGK

The complementarity determining regions (CDRs) of SEQ ID NO: 2 are depicted in bolded and underlined text. The knob in hole T366W mutation (corresponding to IgG4 T246W mutation) in SEQ ID NO: 2 is depicted in underlined text. The S241P (for IgG4 arm swapping, corresponding to S108P) and L248E (for FCR, corresponding to L115E mutation) mutations are depicted in bolded text. In some embodiments, the HVR-H1 of the heavy chain variable domain has the sequence GYHFTSYWMH (SEQ ID NO:9), the HVR-H2 of the heavy chain variable domain has the sequence VIHPNSGSINYNEKFES (SEQ ID NO:10), and the HVR-H3 of the heavy chain variable domain has the sequence ERDSTEVLPMDY (SEQ ID NO:11).

One heavy chain of the single-arm antibody (the heavy chain 1 domain) of the single-arm antibody may comprise the following amino acid sequence (SEQ ID NO:20):

(SEQ ID NO: 20)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQENWYVDGVEVHNAKTKPREEQFNSTYRVVSVLTVLHQDWL
NGKEYKCKVSNKGLPSSIEKTISKAKGQPREPQVYTLPPSQEEMT
KNQVSLWCLVKGFYPSDIAVEWESNGQPENNYKTTPPVLDSDGSF
FLYSRLTVDKSRWQEGNVFSCSVMHEALHNHYTQKSLSLSLGK

The complementarity determining regions (CDRs) of SEQ ID NO: 20 are depicted in bolded and underlined text. The knob in hole T366W mutation (corresponding to IgG4 T246W mutation) in SEQ ID NO: 20 is depicted in underlined text. The S241P (for IgG4 arm swapping, corresponding to $108P) mutation and L248 (corresponding to L115E mutation) are depicted in bolded text.

The antibody may be of the IgG1 class. The sequence of IgG1 heavy chain is

```
(SEQ ID NO: 64)
    1 ASTKGPSVFP LAPSSKSTSG GTAALGCLVK
        DYFPEPVTVS WNSGALTSGV HTFPAVLQSS
  61 GLYSLSSVVT VPSSSLGTQT YICNVNHKPS
        NTKVDKKVEP KSCDKTHTCP PCPAPELLGG
121 PSVFLFPPKP KDTLMISRTP EVTCVVVDVS
        HEDPEVKENW YVDGVEVHNA KTKPREEQYN
181 STYRVVSVLT VLHQDWLNGK EYKCKVSNKA
        LPAPIEKTIS KAKGQPREPQ VYTLPPSRDE
241 LTKNQVSLTC LVKGFYPSDI AVEWESNGQP
        ENNYKTTPPV LDSDGSFFLY SKLTVDKSRW
301 QQGNVFSCSV MHEALHNHYT QKSLSLSPGK
```

The domains of IgG1 are as follow: CH1: 1-98, Hinge: 99-110, CH2: 111-223, and CH3: 224-330. IgG1 may comprise mutations. For example, L117A mutation (for FcR binding), L118A mutation (for FcR binding), T249W mutation (for knob in hole mutation), T249S mutation (for knob in hole mutation), L251A mutation (for knob in hole mutation), and/or Y290V mutation (for knob in hole mutation).

One heavy chain of the single-arm antibody (the heavy chain 1 domain) of the single-arm antibody may comprise the following amino acid sequence (SEQ ID NO: 21):

(SEQ ID NO: 21)
QVQLVQSGAELKKPGASVKVSCKSSGYHETSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPEAAGGPSVFLFPPKPKDTLMISRTP EVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLWCLVKGFYPSDIAVEWESNGQPENNYKTTPPVLDSD
GSFFLYSKLTVDKSRWQQGNVFSCSVMHEALHNHYTQKSLSLSPG
K

The complementarity determining regions (CDRs) of SEQ ID NO: 21 are depicted in bolded and underlined text. The knob in hole T366W mutation (corresponding to IgG1 T249W mutation) in SEQ ID NO: 21 is depicted in underlined text. The L234A (corresponding to IgG1 L117A mutation) and L235A (corresponding to IgG1 L1178 mutation) mutations are depicted in bolded text.

The heavy chain 1 of the single-arm antibody may comprise the following heavy chain variable domain amino acid sequence:

(SEQ ID NO: 15)
QVQLQQPGAELVKPGASVKLSCKSSGYHFTSYWMHWVKQRPGQGL
EWIGVIHPNSGSINYNEKFESKATLTVDKSSSTAYMQLSSLTSED
SAVYYCAGERDSTEVLPMDYWGQGTSVTVSS.

The single-arm antibody may comprise a heavy chain variable domain amino acid sequence that is at least 85%, 90%, or 95% identical to SEQ ID NO: 15, preferably while retaining the HVR-H1 GYHFTSYWMH (SEQ ID NO: 9), the HVR-H2 VIHPNSGSINYNEKFES (SEQ ID NO: 10), and the HVR-H3 ERDSTEVLPMDY (SEQ ID NO: 11).

The single-arm antibody may comprise the following amino acid sequence of heavy chain variable domain variant 1 (VH1):

(SEQ ID NO: 16)
QVQLVQSGAELKKPGASVKVSCKSSGYHFTSYWMHWVKQAPGQGLEWIGVI
HPNSGSINYNEKFESKATITVDKSTSTAYMQLSSLTSEDSAVYYCAGERDS
TEVLPMDYWGQGTSVTVSS

The hyper variable regions (HVRs) of VH1 are depicted in bolded and underlined text.

The single-arm antibody may comprise the following amino acid sequence of heavy chain variable domain variant 2 (VH2):

(SEQ ID NO: 17)
QVQLVQSGAELKKPGASVKVSCKSSGYHFTSYWMHWVKQAPGQGLEWIGVI
HPNSGSINYNEKFESRATITVDKSTSTAYMELSSLRSEDTAVYYCAGERDS
TEVLPMDYWGQGTTVTVSS

The hyper variable regions (HVRs) of VH2 are depicted in bolded and underlined text.

The single-arm antibody may comprise the following amino acid sequence of heavy chain variable domain variant 3 (VH3):

(SEQ ID NO: 18)
QVQLVQSGAELKKPGASVKVSCKSSGYHFTSYWMHWVKQAPGQGLEWIGVI
HPNSGSINYNEKFESRVTITVDKSTSTAYMELSSLRSEDTAVYYCAGERDS
TEVLPMDYWGQGTTVTVSS.

The hyper variable regions (HVRs) of VH3 are depicted in bolded and underlined text.

The single-arm antibody may comprise the following amino acid sequence of heavy chain variable domain variant 4 (VH4):

(SEQ ID NO: 19)
QVQLVQSGAELKKPGASVKVSCKSSGYHFTSYWMHWVRQAPGQGLEWIGVI
HPNSGSINYNEKFESRVTITVDKSTSTAYMELSSLRSEDTAVYYCAGERDS
TEVLPMDYWGQGTTVTVSS

The hyper variable regions (HVRs) of VH4 are depicted in bolded and underlined text.

The single-arm antibody may comprise a heavy chain variable domain amino acid sequence that is at least 85%, 90%, or 95% identical to SEQ ID NO: 16-19 while retaining the HVR-H1 GYHFTSYWMH (SEQ ID NO: 9), the HVR-H2 VIHPNSGSINYNEKFES (SEQ ID NO: 10), and the HVR-H3 ERDSTEVLPMDY (SEQ ID NO: 11).

A second heavy chain of the single-arm antibody (the heavy chain 2 domain) of the single-arm antibody, which is N-terminally truncated, may comprise the following amino acid sequence (SEQ ID NO: 3):

(SEQ ID NO: 3)
ESKYGPPCPPCPAPEFEGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLSCAVKGFYPSDIAVEWESNGQPENNYKTTPPVL
DSDGSFFLVSRLTVDKSRWQEGNVFSCSVMHEALHNHYTQKSLSL
SLGK

There is no heavy chain variable domain and no CDRs in SEQ ID NO: 3. The knob in hole T366S/L368A/Y407V mutations in SEQ ID NO: 3 are depicted in underlined text. The S241P and L248E mutations are depicted in bolded text.

A second heavy chain of the single-arm antibody (the heavy chain 2 domain) of the single-arm antibody, which is N-terminally truncated, may comprise the following amino acid sequence (SEQ ID NO: 42):

(SEQ ID NO: 42)
ESKYGPPCPPCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSTEKTISKAKGQPREPQVYTLPP
SQEEMTKNQVSLSCAVKGFYPSDIAVEWESNGQPENNYKTTPPVL
DSDGSFFLVSRLTVDKSRWQEGNVFSCSVMHEALHNHYTQKSLSL
SLGK

A second heavy chain of the single-arm antibody (the heavy chain 2 domain) of the single-arm antibody, which is N-terminally truncated, may comprise the following amino acid sequence (SEQ ID NO: 43):

(SEQ ID NO: 43)
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLSCAVKGFYPSDIAVEWESNGQPENNYKTTPPVLDS
DGSFFLVSKLTVDKSRWQQGNVFSCSVMHEALHNHYTQKSLSLSP
GK

There is no heavy chain variable domain and no CDRs in SEQ ID NO: 43. The knob in hole T366S/L368A/Y407V mutations in SEQ ID NO: 43 are depicted in underlined text. The L234A and L235A mutations are depicted in bolded text.

A second heavy chain of the antibody
(the heavy chain 2 domain) may comprise any
one of the following amino acid sequences
(SEQ ID NOs: 44-49, 65):
(SEQ ID NO: 44)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGKRKSTKVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 45)
QVQLVQSGAELKKPGASVKVSCKSSGYHFTSYWMKWVKQAPGQGL
EWIGVIHPNSGSINYNKKFESRVTITVDKSTSTAYMELSSLRSED
TAVYYCAGARKSTEVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 46)
QVQLVQSGAELKKPGASVKVSCKSSGYHFTSYWMHWVKQAPGQGL
EWIGVIHPNSGSINYNKKFKSRVTITVDKSTSTAYMELSSLRSED
TAVYYCAGKRKSTKVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 65)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGARASTAVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
A second heavy chain of the antibody (the
heavy chain 2 domain) may comprise any
one of the following amino acid sequences
(SEQ ID NOs: 48-51):
(SEQ ID NO: 48)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGKRKSTKVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 49)
QVQLVQSGAELKKPGASVKVSCKSSGYHFTSYWMKWVKQAPGQGL
EWIGVIHPNSGSINYNKKFESRVTITVDKSTSTAYMELSSLRSED
TAVYYCAGARKSTEVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 50)
QVQLVQSGAELKKPGASVKVSCKSSGYHFTSYWMHWVKQAPGQGL
EWIGVIHPNSGSINYNKKFKSRVTITVDKSTSTAYMELSSLRSED
TAVYYCAGKRKSTKVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK
(SEQ ID NO: 51)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGARASTAVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLSCAVKGFYPSDIAVEWESNGQPENNYKTTPPVLDSDGSF
FLVSRLTVDKSRWQEGNVFSCSVMHEALHNHYTQKSLSLSLGK

The CDRs in the second heavy chain variable domain are mutated in SEQ ID NOs: 44-51 to prevent binding to C1q. The CDR mutations are depicted in bolded and underlined text. The knob in hole T366S/L368A/Y407V mutations in SEQ ID NOs: 44-51 are depicted in underlined text.

In some embodiments, the antibody that binds to C1q, comprising:
a light chain domain comprising the amino acid sequence of SEQ ID NO: 40;
a first heavy chain domain comprising the amino acid sequence of SEQ ID NO: 2; and
a second heavy chain domain comprising the amino acid sequence of SEQ ID NO: 3; the second heavy chain domain is an N-terminally truncated heavy chain.

### C1s Inhibitors

The inhibitor of the classical complement pathway may be a C1s inhibitor, such as a small molecule, an antibody, an aptamer, an antisense nucleic acid or a gene editing agent.

Exemplary C1s small molecule inhibitors are described in U.S. patent application Ser. No. 17/379,334 and U.S. patent application Ser. No. 18/097,811, the contents of which are incorporated herein by reference.

### Anti-Complement C1s Antibodies

Suitable inhibitors include an antibody that binds complement C1s protein (i.e., an anti-complement C1s antibody, also referred to herein as an anti-C1s antibody and a C1s antibody) and a nucleic acid molecule that encodes such an antibody. Complement C1s is an attractive target as it is upstream in the complement cascade and has a narrow range of substrate specificity. Furthermore it is possible to obtain antibodies (for example, but not limited to, monoclonal antibodies) that specifically bind the activated form of C1s.

All sequences mentioned in the following two paragraphs are incorporated by reference from U.S. patent application Ser. No. 14/890,811, which is hereby incorporated by reference for the antibodies and related compositions that it discloses.

In certain aspects, disclosed herein are methods of administering an anti-C1s antibody. The antibody may be a murine, humanized, or chimeric antibody. In some embodiments, the light chain variable domain comprises HVR-L1, HVR-L2, and HVR-L3, and the heavy chain comprises HVR-H1, HVR-H2, and HVR-H3 of a murine anti-human C1s monoclonal antibody 5A1 produced by a hybridoma cell line deposited with ATCC on May 15, 2013 or progeny thereof (ATCC Accession No. PTA-120351). In other embodiments, the light chain variable domain comprises the HVR-L1, HVR-L2, and HVR-L3 and the heavy chain variable domain comprises the HVR-H1, HVR-H2, and HVR-H3 of a murine anti-human C1s monoclonal antibody 5C12 produced by a hybridoma cell line deposited with ATCC on May 15, 2013, or progeny thereof (ATCC Accession No. PTA-120352).

In some embodiments, antibodies specifically bind to and inhibit a biological activity of C1s or the C1s proenzyme, such as C1s binding to C1q, C1s binding to C1r, or C1s binding to C2 or C4. The biological activity may be a proteolytic enzyme activity of C1s, the conversion of the C1s proenzyme to an active protease, or proteolytic cleavage of C2 or C4. In certain embodiments, the biological activity is activation of the classical complement activation pathway, activation of antibody and complement dependent cytotoxicity, or CIF hemolysis.

All anti-C1s antibody sequences are incorporated by reference from U.S. Pat. No. 8,877,197, which is hereby incorporated by reference for the antibodies and related compositions that it discloses.

In some embodiments, an anti-C1s antibody of the present disclosure (e.g., a subject antibody that specifically binds an epitope in a complement C1s protein) comprises: a) a light chain region comprising CDRs selected from SEQ ID NO:54, SEQ ID NO:55, and SEQ ID NO: 56; and b) a heavy chain region comprising CDRs selected from SEQ ID NO:57, SEQ ID NO: 58, and SEQ ID NO:59. In some of these embodiments, the anti-C1s antibody includes a humanized VH and/or VL framework region.

SEQ ID NO: 54: SSVSSSYLHWYQ;
SEQ ID NO: 55: STSNLASGVP;
SEQ ID NO: 56: HQYYRLPPIT;
SEQ ID NO: 57: GFTFSNYAMSWV;
SEQ ID NO: 58: ISSGGSHTYY;
SEQ ID NO: 59: ARLFTGYAMDY.

In some embodiments, an anti-C1s antibody of the present disclosure comprises a light chain variable region comprising amino acid sequence SEQ ID NO:21.

In some embodiments, an anti-C1s antibody of the present disclosure comprises a heavy chain variable region comprising amino acid sequence SEQ ID NO:22.

(SEQ ID NO: 60)
DIVMTQTTAIMSASLGERVTMTCTASSSVSSSYLHWYQQKPGSS
PKLWIYSTSNLASGVPARFSGSGSGTFYSLTISSMEAEDDATYY
CHQYYRLPPITFGAGTKLELK
(SEQ ID NO: 22)
QVKLEESGGALVKPGGSLKLSCAASGFTFSNYAMSWVRQIPEKR
LEWVATISSGGSHTYYLDSVKGRFTISRDNARDTLYLQMSSLRS
EDTALYYCARLFTGYAMDYWGQGTSVT

In some embodiments, an anti-C1s antibody of the present disclosure comprises a light chain variable region comprising amino acid sequence SEQ ID NO:23.

In some embodiments, an anti-C1s antibody of the present disclosure comprises a heavy chain variable region comprising amino acid sequence SEQ ID NO:24.

(SEQ ID NO: 23)
QIVLTQSPATLSLSPGERATMSCTASSSVSSSYLHWYQQKPGKAPKLWIY
STSNLASGVPSRFSGSGSGTDYTLTISSLQPEDFATYYCHQYYRLPPITF
GQGTKLEIK
(SEQ ID NO: 24)
EVQLVESGGGLVKPGGSLRLSCAASGFTFSNYAMSWVRQAPGKGLEWVAT
ISSGGSHTYYLDSVKGRFTISRDNSKNTLYLQMNSLRAEDTALYYCARLF
TGYAMDYWGQGTLVTVSS

In some embodiments, an anti-C1s antibody of the present disclosure comprises a light chain comprising amino acid sequence SEQ ID NO:25.

In some embodiments, an anti-C1s antibody of the present disclosure comprises a heavy chain comprising amino acid sequence SEQ ID NO:26.

Sutimlimab antibody comprises a light chain comprising amino acid sequence SEQ ID NO: 25 and a heavy chain comprising amino acid sequence SEQ ID NO:26.

```
  (SEQ ID NO: 25)
QIVLTQSPATLSLSPGERATMSCTASSSVSSSYLHWYQQKPGKAPKLWIY
STSNLASGVPSRFSGSGSGTDYTLTISSLQPEDFATYYCHQYYRLPPITF
GQGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
  (SEQ ID NO: 26)
EVQLVESGGGLVKPGGSLRLSCAASGFTFSNYAMSWVRQAPGKGLEWVAT
ISSGGSHTYYLDSVKGRFTISRDNSKNTLYLOMNSLRAEDTALYYCARLF
TGYAMDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
```

In some embodiments, an anti-C1s antibody of the present disclosure comprises: a) a light chain region comprising CDRs selected from SEQ ID NO:27, SEQ ID NO:28, and SEQ ID NO: 56; and b) a heavy chain region comprising CDRs selected from SEQ ID NO:29, SEQ ID NO: 30, and SEQ ID NO:61. In some of these embodiments, the anti-C1s antibody includes a humanized VH and/or VL framework region.

SEQ ID NO: 27: TASSSVSSSYLH;
SEQ ID NO: 28: STSNLAS;
SEQ ID NO: 56: HQYYRLPPIT;
SEQ ID NO: 29: NYAMS;
SEQ ID NO: 30: TISSGGSHTYYLDSVKG;
SEQ ID NO: 61: LFTGYAMDY.

In some embodiments, an anti-C1s antibody of the present disclosure comprises a light chain variable region comprising amino acid sequence SEQ ID NO:32.

In some embodiments, an anti-C1s antibody of the present disclosure comprises a heavy chain variable region comprising amino acid sequence SEQ ID NO:33.

(SEQ ID NO: 62)
QIVLTQSPAIMSASLGERVTMTCTASSSVSSSYLHWYQQKPGSSPKLWIY
STSNLASGVPARFSGSGSGTFYSLTISSMEAEDDATYYCHQYYRLPPITF
GAGTKLELK
(SEQ ID NO: 63)
EVMLVESGGALVKPGGSLKLSCAASGFTFSNYAMSWVRQIPEKRLEWVAT
ISSGGSHTYYLDSVKGRFTISRDNARDTLYLQMSSLRSEDTALYYCARLF
TGYAMDYWGQGTSVTVSS

The anti-C1s antibody may be selected from an antigen binding fragment, Ig monomer, a Fab fragment, a F(ab′)₂ fragment, a Fd fragment, a scFv, a scAb, a dAb, a Fv, a single domain heavy chain antibody, a single domain light chain antibody, a mono-specific antibody, a bi-specific antibody, or a multi-specific antibody.

Disclosed herein are methods of administering an antibody that competes for binding the epitope bound by antibody IPN003 (also referred to herein as “IPN-M34” or “M34” or “TNT003”), e.g., an antibody comprising a variable domain of antibody IPN003, such as antibody IPN003.

All anti-C1s antibody sequences are incorporated by reference from U.S. patent application Ser. No. 17/749,362, which is hereby incorporated by reference for the antibodies and related compositions that it discloses.

In some embodiments, an anti-C1s antibody of the present disclosure comprises (1) a heavy chain variable region comprising a HCDR1, a HCDR2, and a HCDR3, and (2) a light chain variable region comprising a LCDR1, a LCDR2, and a LCDR3, wherein:
(a) the HCDR1 comprises the amino acid sequence selected from the group consisting of SEQ ID NO: 66, 72, 78, 84, 89, 94, 99, 103, and 107,
(b) the HCDR2 comprises the amino acid sequence selected from the group consisting of SEQ ID NO: 67, 73, 79, 85, 90, 95, 100, 104, or 108,
(c) the HCDR3 comprises the amino acid sequence selected from the group consisting of SEQ ID NO: 68, 74, 80, 101, 105, or 109,
(d) the LCDR1 comprises the amino acid sequence selected from the group consisting of SEQ ID NO: 69, 75, 81, 86, 91, 96, 102, 106, or 110,
(e) the LCDR2 comprises the amino acid sequence selected from the group consisting of SEQ ID NO: 70, 76, 82, 87, 92, or 97, and
(f) the LCDR3 comprises the amino acid sequence selected from the group consisting of SEQ

TABLE 2
Kabat CDRs

| Ab ID | | | | | | |
|---|---|---|---|---|---|---|
| No | HCDR1 | HCDR2 | HCDR3 | LCDRI | LCDR2 | LCDR3 |
| MAb1 | ELSMH | TFDPEEGET | EGLAGRPEDS | RASQSISSW | KASSLES | QQYNSYSWT |
| | SEQ ID | IYAQKFQG | SEQ ID | LA | (SEQ ID | (SEQ ID |
| | NO: 66) | (SEQ ID | NO: 68) | (SEQ ID | NO: 70) | NO: 71) |
| | | NO: 67) | | NO: 69) | | |
| MAb2 | DYYMS | YISRSGSTKY | DETDYALDY | QASQDISNY | DASNLET | QQYEDLPLT |
| | (SEQ ID | YADSVKG | SEQ ID | LN (SEQ | (SEQ ID | (SEQ ID |
| | NO: 72) | (SEQ ID | NO: 74) | ID NO: 75) | NO: 76) | NO: 77) |
| | | NO: 73) | | | | |
| MAb3 | DYGMS | GINWEGGST | DEQLGGNYY | RASQGIRN | TASNLQS | LQYNSYPLT |
| | (SEQ ID | GYADSVKG | YYYYMDV | DLG | (SEQ ID | (SEQ ID |
| | NO: 78) | (SEQ ID | SEQ ID | (SEQ ID | NO: 82) | NO: 83) |
| | | NO: 79) | NO: 80) | NO: 81) | | |
| MAb1 | GDTLTEL | DPEEGE | EGLAGRPFDS | SQSISSW | KAS | YNSYSW |
| | (SEQ ID | (SEQ ID | (SEQ ID | (SEQ ID | (SEQ ID | (SEQ ID |
| | NO: 84) | NO: 85) | NO: 68) | NO: 86) | NO: 87) | NO: 88) |
| MAb2 | GFTFSDY | SRSGST | DETDYALDY | SQDISNY | DAS | YEDLPL |
| | (SEQ ID | (SEQ ID | (SEQ ID | (SEQ ID | (SEQ ID | ( SEQ ID |
| | NO: 89) | NO: 90) | NO: 74) | NO: 91) | NO: 92) | NO: 93) |
| MAb3 | GFTEDDY | NWEGGS | DEQLGGNYY | SQGIRND | TAS | YNSYPL |
| | SEQ ID | (SEQ ID | YYYYMDV | (SEQ ID | (SEQ ID | (SEQ ID |
| | NO: 94) | NO: 95) | (SEQ ID | NO: 96) | NO: 97) | NO: 98) |
| | | | NO: 80) | | | |
| MAb1 | GDTLTEL | FDPEEGET | VTEGLAGRPF | QSISSW | KAS | QQYNSYSWT |
| | S | (SEQ ID | DS | (SEQ ID | (SEQ ID | ((SEQ ID |
| | (SEQ ID | NO: 100) | (SEQ ID | NO: 102) | NO: 87) | NO: 71) |
| | NO: 99) | | NO: 101) | | | |
| MAb2 | GFTFSDY | ISRSGSTK | ARDETDYAL | QDISNY | DAS | QQYEDLPLT |
| | Y | (SEQ ID | DY | (SEQ ID | (SEQ ID | (SEQ ID |
| | (SEQ ID | NO: 104) | (SEQ ID | NO: 106) | NO: 92) | NO: 77) |
| | NO: 103) | | NO: 105) | | | |
| MAb3 | GFTFDDY | INWEGGST | ARDEQLGGN | QGIRND | TAS | LQYNSYPLT |
| | G | (SEQ ID | YYYYYYMDV | (SEQ ID | (SEQ ID | (SEQ ID |
| | SEQ ID | NO: 108) | (SEQ ID | NO: 110) | NO: 97) | NO: 83) |
| | NO: 107) | | NO: 109) | | | |

### C1r Inhibitors

The inhibitor of the classical complement pathway may be a C1r inhibitor, such as a small molecule, an antibody, an aptamer, an antisense nucleic acid or a gene editing agent.

The anti-C1r antibodies disclosed herein are potent inhibitors of C1r.

The anti-C1r antibodies disclosed herein inhibit the interaction between C1r and C1q or between C1r and C1s, or wherein the anti-C1r antibody inhibits the catalytic activity of C1r or inhibits the processing of pro-C1r to an active protease.

### C1 Complex Inhibitors

The inhibitor of the classical complement pathway may be a C1 complex inhibitor, such as a small molecule, an antibody, an aptamer, an antisense nucleic acid or a gene editing agent.

The anti-C1 complex antibodies disclosed herein are potent inhibitors of C1 complex.

The anti-C1 complex antibodies disclosed herein inhibit C1r or C1s activation or blocks their ability to act on C2 or C4. The anti-C1 complex antibodies disclosed herein bind to a combinatorial epitope within the C1 complex, wherein said combinatorial epitope comprises amino acids of both C1q and C1s; both C1q and C1r; both C1r and C1s; or each of C1q, C1r, and C1s.

### C2 Inhibitors

The inhibitor of the classical complement pathway may be a C2 inhibitor, such as a small molecule, an antibody, an aptamer, an antisense nucleic acid or a gene editing agent.

The anti-C2 inhibitors disclosed herein are potent inhibitors of C2. In some embodiments, the C2 inhibitor is ARGX-117 (ArgenX) described in U.S. Pat. No. 11,161,900, the contents of which are incorporated herein by reference. All anti-C2 antibody sequences are incorporated by reference from U.S. Pat. No. 11,161,900, which is hereby incorporated by reference for the antibodies and related compositions that it discloses.

In some embodiments, an anti-C2 antibody of the present disclosure comprises (1) a heavy chain variable region comprising a HCDR1, a HCDR2, and a HCDR3, and (2) a light chain variable region comprising a LCDR1, a LCDR2, and a LCDR3, wherein: the HCDR1 comprises the amino acid sequence of SEQ ID NO: 111 (DYNMD), the HCDR2 comprises the amino acid sequence of SEQ ID NO: 112 (DINPNYESTGYNQKFKG), the HCDR3 comprises the amino acid sequence of SEQ ID NO: 113 (EDDHDAFAY), the LCDR1 comprises the amino acid sequence of SEQ ID NO: 114 (RASKSVRTSGYNYMH), the LCDR2 comprises the amino acid sequence of SEQ ID NO: 115 (LASNLKS), the LCDR3 comprises the amino acid sequence of SEQ ID NO: 116 (QHSRELPYT),

In some embodiments, an anti-C2 antibody of the present disclosure comprises a VH domain comprising the amino acid sequence set forth in SEQ ID NO: 118; and a VL domain comprising the amino acid sequence set forth in SEQ ID NO: 117.

(SEQ ID NO: 117)
DNVLTQSPDSLAVSLGERATISCRASKSVRTSGYNYMHWYQQKPGQPPKL
LIYLASNLKSGVPDRFSGSGSGTDFTLTISSLQAEDAATYYCQHSRELPY
TFGQGTKLEIK
(SEQ ID NO: 118)
EVQLVQSGAEVKKPGASVKVSCKASGYTFTDYNMDWVRQATGQGLEWIGD
INPNYESTGYNQKFKGRATMTVDKSISTAYMELSSLRSEDTAVYYCARED
DHDAFAYWGQGTLVTVSS

### C3 Inhibitors

The inhibitor of the classical complement pathway may be a C3 inhibitor, such as a small molecule, an antibody, an aptamer, an antisense nucleic acid or a gene editing agent.

The anti-C3 inhibitors disclosed herein are potent inhibitors of C3. In some embodiments, the C3 inhibitor is APL-9 (Apellis) and/or AMY-101 (Amyndas) and/or IVT CB 2782-PEG (Catalyst Biosciences and Biogen).

### C4 Inhibitors

The inhibitor of the classical complement pathway may be a C4 inhibitor, such as a small molecule, an antibody, an aptamer, an antisense nucleic acid or a gene editing agent.

The anti-C4 inhibitors disclosed herein are potent inhibitors of C4.

### Inhibition of Complement

A number of molecules are known that inhibit the activity of complement. In addition to known compounds, suitable inhibitors can be screened by methods described herein. As described above, normal cells can produce proteins that block complement activity, e.g., CD59, C1 inhibitor, etc. In some embodiments of the disclosure, complement is inhibited by upregulating expression of genes encoding such polypeptides.

Modifications of molecules that block complement activation are also known in the art. For example, such molecules include, without limitation, modified complement receptors, such as soluble CR1. The mature protein of the most common allotype of CR1 contains 1998 amino acid residues: an extracellular domain of 1930 residues, a transmembrane region of 25 residues, and a cytoplasmic domain of 43 residues. The entire extracellular domain is composed of 30 repeating units referred to as short consensus repeats (SCRs) or complement control protein repeats (CCPRs), each consisting of 60 to 70 amino acid residues. Recent data indicate that C1q binds specifically to human CR1. Thus, CR1 recognizes all three complement opsonins, namely C3b, C4b, and C1q. A soluble version of recombinant human CR1 (sCR1) lacking the transmembrane and cytoplasmic domains has been produced and shown to retain all the known functions of the native CR1. The cardioprotective role of sCR1 in animal models of ischemia/reperfusion injury has been confirmed. Several types of human C1q receptors (C1qR) have been described. These include the ubiquitously distributed 60- to 67-kDa receptor, referred to as cC1qR because it binds the collagen-like domain of C1q. This C1qR variant was shown to be calreticulin; a 126-kDa receptor that modulates monocyte phagocytosis. gC1qR is not a membrane-bound molecule, but rather a secreted soluble protein with affinity for the globular regions of C1q, and may act as a fluid-phase regulator of complement activation.

Decay accelerating factor (DAF) (CD55) is composed of four SCRs plus a serine/threonine-enriched domain that is capable of extensive O-linked glycosylation. DAF is attached to cell membranes by a glycosyl phosphatidyl inositol (GPI) anchor and, through its ability to bind C4b and C3b, it acts by dissociating the C3 and C5 convertases. Soluble versions of DAF (sDAF) have been shown to inhibit complement activation.

C1 inhibitor, a member of the “serpin” family of serine protease inhibitors, is a heavily glycosylated plasma protein that prevents fluid-phase C1 activation. C1 inhibitor regulates the classical pathway of complement activation by blocking the active site of C1r and C1s and dissociating them from C1q.

Peptide inhibitors of complement activation include C5a and other inhibitory molecules include Fucan.

### Methods of Treatment

The present disclosure is generally directed to methods of treating lupus nephritis in a subject in need thereof. The method comprises determining that the subject has at least one of the following characteristics: an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. C4x may be selected from C4a and C4d. The method further comprises administering to the subject an inhibitor of the classical complement pathway, e.g., if the subject has an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. In some embodiments, the subject has at least two of the characteristics. For example, the characteristics may be selected from elevated C4x levels, elevated C4x/C4 ratio and reduced C4 levels, and/or the characteristics may be selected from the group consisting of elevated C1sC1 inhibitor levels, elevated C1sC1 inhibitor/C1s ratio and reduced C1s levels. In some embodiments, the subject further has an elevated Pathogenic Anti-C1q Antibody 1 (PACA1) and/or Pathogenic Anti-C1q Antibody 3 (PACA3) level. In some embodiments, the subject has at least one of the following additional characteristics: an elevated C3a level; an elevated C3a/C3 ratio; or a reduced C3 level. In some embodiments, the subject has elevated UPCR as yet another additional characteristic. A therapeutically effective amount of the inhibitor may be administered.

We observed evidence of complement activation in lupus nephritis patients relative to healthy controls. Specifically, levels of C4a, C4d, C1sC1 inhibitor, PACA1, and PACA3, and the C4a/C4, C4d/C4, C1sC1 inhibitor/C1s, C2b/C2 and C3a/C3 ratios were highly increased in lupus nephritis patients, while levels of C4, C1s, C2, and C3 were decreased in lupus nephritis patients. More specifically, we observed levels of C4d and the C4d/C4 ratio were highly increased in lupus nephritis patients with flare, while levels of C1q, C1s, C2 and C4 were decreased, consistent with activation of the classical complement pathway (increased activation and component consumption).

Data from Example 1 demonstrates that patients identified via the use of a model evaluating elevated C4d levels and/or elevated C4d/C4 ratios exhibit potential clinical improvement with anti-C1q antibody treatment. Moreover, these data suggest that a subset of LN patients exhibiting high levels of C4a, C4d, C1sC1 inhibitor, PACA1, PACA3, C4a/C4, are C1sC1 inhibitor/C1s, and C2b/C2 or reduced levels of C4, C1s, C2 are also likely to benefit from therapy with a classical complement inhibitor, or more specifically an anti-C1q antibody therapy. Treatment with an anti-C1q antibody (such as a full-length anti-C1q antibody or Fab fragment thereof) leads to clinical benefits in lupus nephritis patients identified by the use of a model evaluating complement levels and/or PACA levels alone or in combination with UPCR as shown in Example 4. Accounting for complement levels (e.g., C4a, C4d, C4, C2b, C2, C1sC1 inhibitor, C1s, C3a, C3a, C3, PACA1, PACA3, and the C4a/C4, C4d/C4, C1sC1 inhibitor/C1s, C2b/C2 and C3a/C3 ratios) and/or PACA levels alone or in combination with UPCR, and the use of a classical complement inhibitor and more specifically, an anti-C1q antibody, including an anti-C1q antibody fragment (e.g., Fab), reliably leads to clinical benefits in lupus nephritis patients.

Systemic lupus erythematosus (SLE) is a biologically and clinically heterogeneous autoimmune disease that is characterized by a broad spectrum of diverse clinical involvement, particularly kidney involvement. Renal involvement in SLE is termed LN. LN occurs in up to 50% to 60% of SLE patients within the first 10 years of the disease and is a major cause of morbidity and mortality in SLE.

Class III (focal proliferative) and Class IV (diffuse proliferative) LN according to the International Society of Nephrology/Renal Pathology Society (ISN/RPS) clinic-pathologic classification system are considered more severe, carry a poorer prognosis than Class I (minimal disease) or Class II (mesangial proliferative) disease, and warrants treatment with potent immune-suppressants such as cyclophosphamide, mycophenolate mofetil (MMF), and high doses of glucocorticoids. Both Class III and IV LN may have active (proliferative), inactive (sclerosing), or combined active and inactive lesions. Additionally, Class V (membranous) glomerulonephritis may occur independently or co-exist with Class III and IV LN.

There remains a large unmet need for new treatment options in LN that are safe and effective with the goal of providing rapid and durable clinical benefit. While guidelines have been developed worldwide for the management of LN, it is currently unknown which therapies are best suited for individual patients. Therefore, a precision medicine approach is warranted to identify patients who may respond better to certain therapies, and to avoid exposure to potentially harmful agents that are unlikely to provide benefit.

It has been estimated that roughly 5 million people worldwide have some form of lupus, with LN affecting roughly half of SLE patients during their lifetime. LN, and particularly Class III/IV proliferative glomerulonephritis, is a serious and life-threatening disease that is associated with a 6-fold increase in mortality compared with the general population. In addition, lupus patients who develop ESRD have a 26-fold increase in the risk of death, more than twice the risk associated with malignancy or cardiovascular disease. LN conveys >2-fold increased mortality risk versus extra-renal SLE.

Class III/IV LN is typically treated with immunosuppressant agents such as cyclophosphamide, MMF, azathioprine, and glucocorticoids, and calcineurin inhibitors and rituximab may be used for refractory cases. All of these agents carry the potential for significant toxicity, and while 50% to 80% of patients receiving these standard treatments may achieve a renal response at 1 year, most of these responses are in only partial remission. Further, as many as 30% of LN patients will progress to ESRD despite receiving standard treatments. Two advanced therapies, belimumab and voclosporin have recently been approved in Class III/IV LN for use in addition to standard treatment, yet only ˜30% to 40% of patients achieved complete renal response with chronic administration of these agents in pivotal trials. There remains a significant unmet need for the development of safe and effective therapies in LN that are tailored for these patients.

Autoantibodies to various self-antigens, such as double-stranded DNA, RNA, and nucleosomes, are believed to contribute to the pathogenesis of lupus. In addition, autoantibodies targeting the collagen-like stalk domain of C1q are highly enriched in LN patients, and may amplify complement-mediated inflammation in LN, and are associated with disease flare. Autoantibodies bind to their cognate antigens to form immune complexes which deposit in the vascular wall, glomeruli, and interstitium of the kidney in LN. Immune complexes trigger activation of the classical complement cascade, with C1q acting as the most proximal step in this pathway. Through a series of recruitment steps and enzymatic reactions, C1q and its downstream components such as C4, C3, and their activation products deposit in the kidney tissues of most LN patients. These signaling events result in activation of antigen-presenting cells, release of inflammatory mediators, and recruitment of additional innate and adaptive immune cells, leading to disruption of the structure and function of the glomerular basement membrane (GBM), development of proteinuria, and ensuing tissue damage. Pathogenic anti-C1q autoantibodies (PACAs) may amplify classical complement pathway-mediated inflammation even further, by binding to C1q associated with immune complexes present in kidney tissue, thereby triggering even more complement activation (FIG. 2). Importantly, while PACAs bind to the tail region of substrate bound C1q, FabA recognizes the head region of C1q and prevents C1q from binding to its substrate in the first place. Therefore, FabA not only inhibits the most proximal step in classical complement pathway activation, but also disrupts PACA-mediated classical pathway amplification.

Complement factor C4 and its activation product C4d are important biomarkers in LN. Reduced levels of C4 and increased amounts of C4d in the circulation signal activation of the classical complement pathway and are associated with active LN. C4d levels in plasma are strongly associated with C4d deposition in kidney tissue from active LN patients. C4d/C4 ratio is elevated in active LN, compared with SLE patients without LN, patients with autoimmune kidney disease IgA nephropathy, and healthy controls. Further, C4d/C4 ratio accurately predicts the presence of active LN to a higher degree than other biomarkers (e.g., C3, C4, and C4d). C4d/C4 may therefore identify active LN patients who are most likely to respond to therapies targeting the classical complement cascade. Similarly, as shown herein, LN patients have an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level.

Example 1 shows that the C4d/C4 ratio also correlated with levels of autoantibodies against C1q isotypes 1 and 3 that are known to activate the classical pathway. These data indicated that a subset of LN patients exhibiting high C4d/C4 ratio along with specific markers of classical pathway activation are likely to benefit from a classical complement inhibitor, or more specifically an anti-C1q therapy. Moreover, these data suggest that a subset of LN patients exhibiting high levels of C4a, C4d, C1sC1 inhibitor, PACA1, PACA3, C4a/C4, are gC1sC1 inhibitor/C1s, and C2b/C2 or reduced levels of C4, C1s, C2 are likely to benefit from therapy with a classical complement inhibitor, or more specifically an anti-C1q therapy.

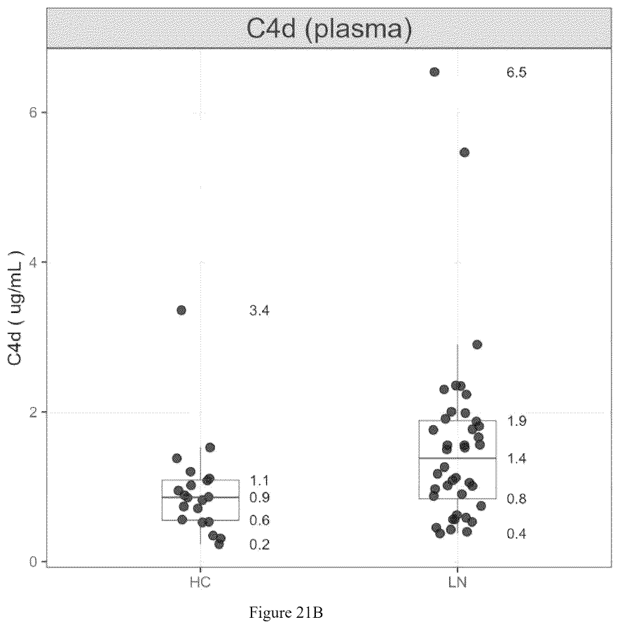

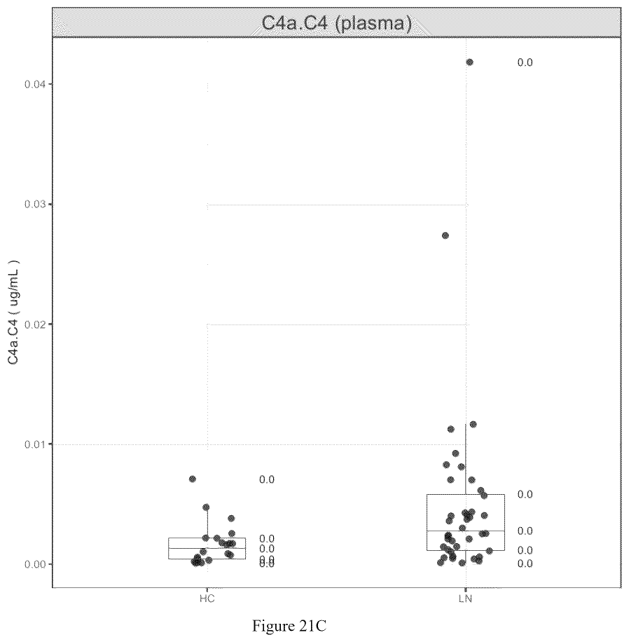

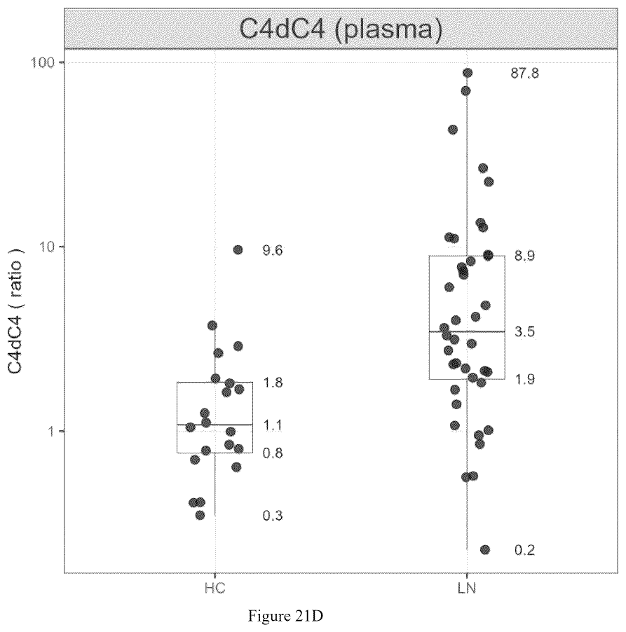

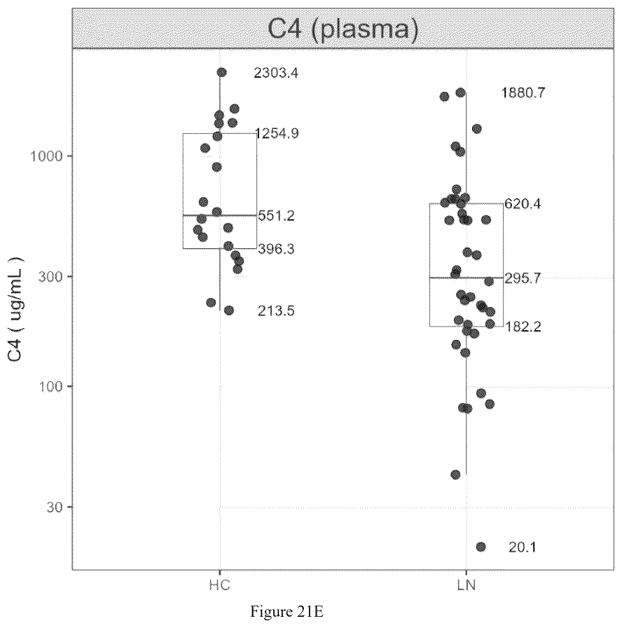

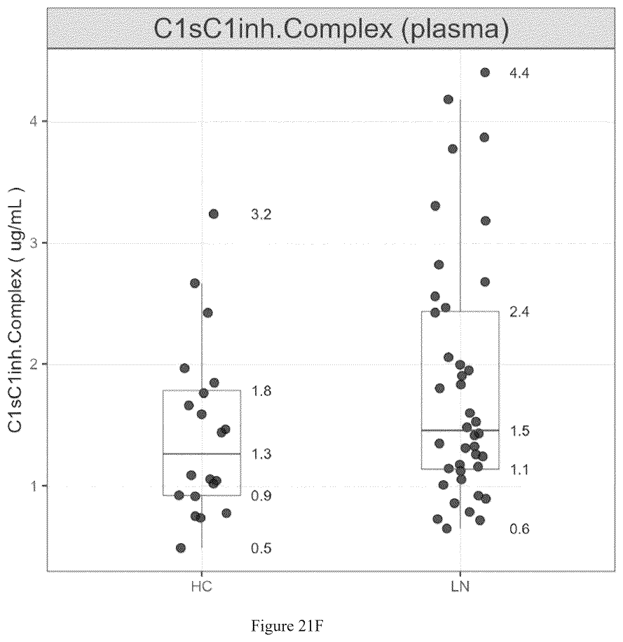

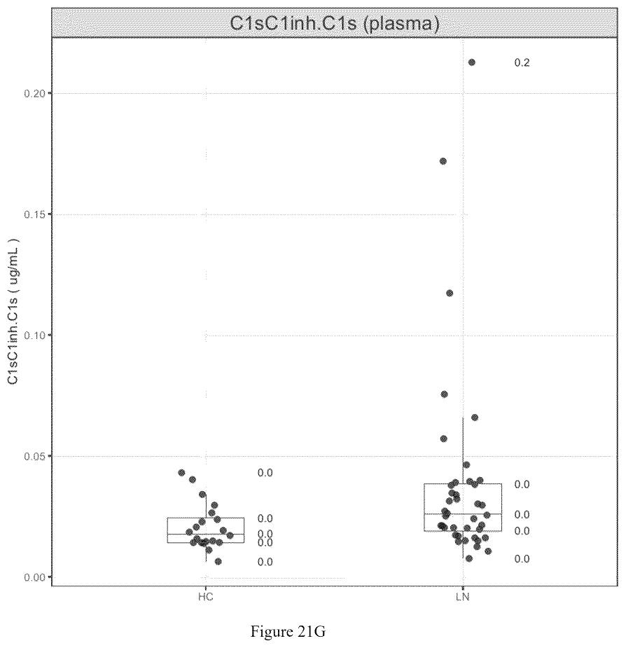

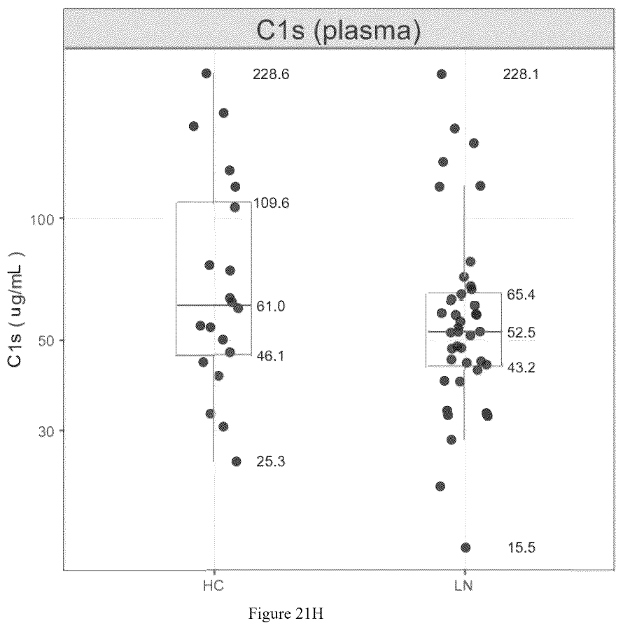

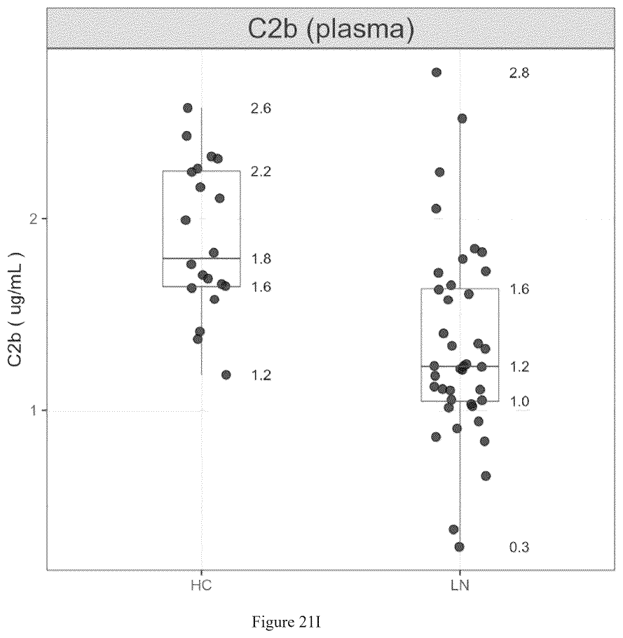

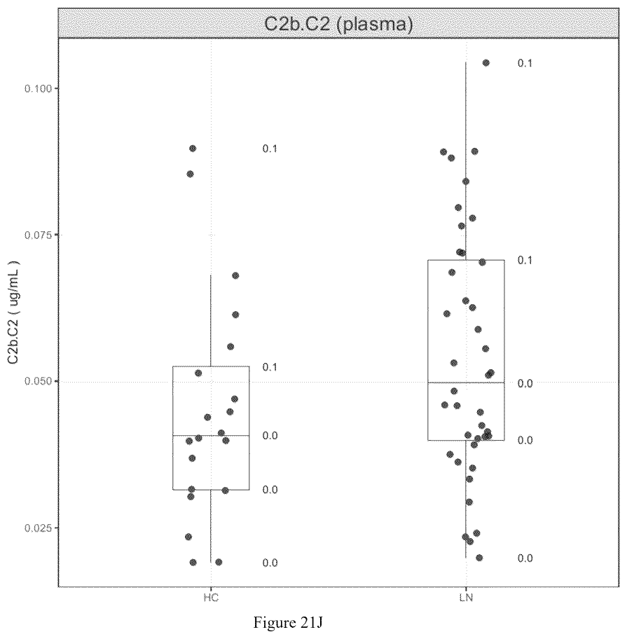

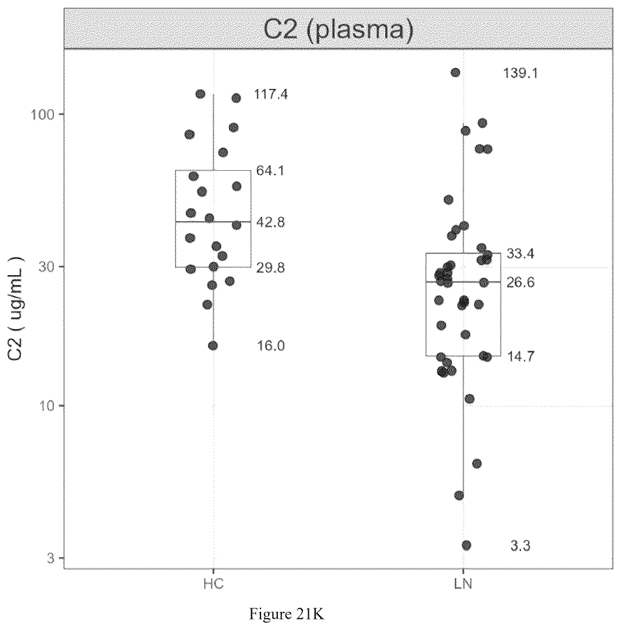

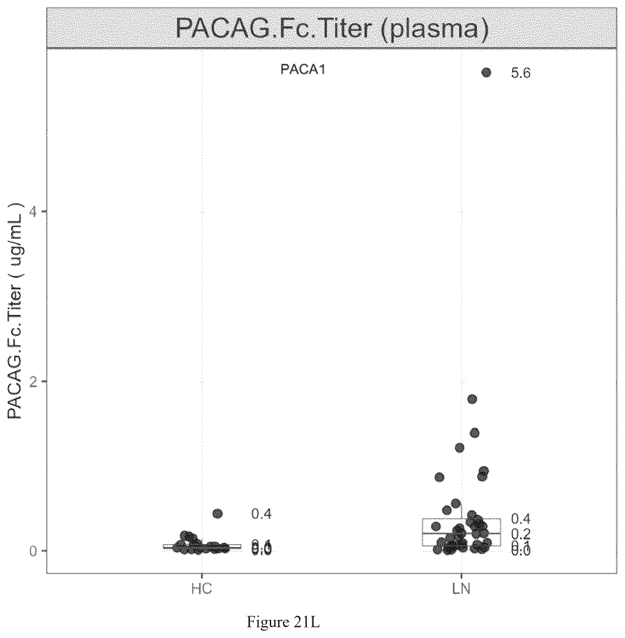

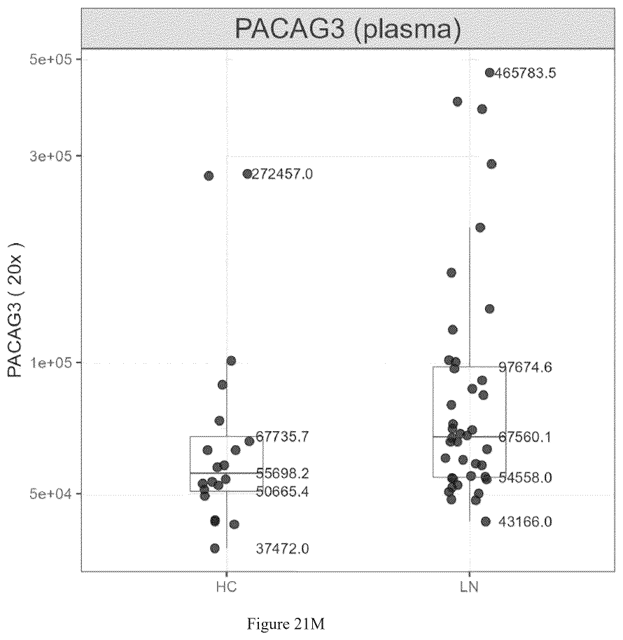

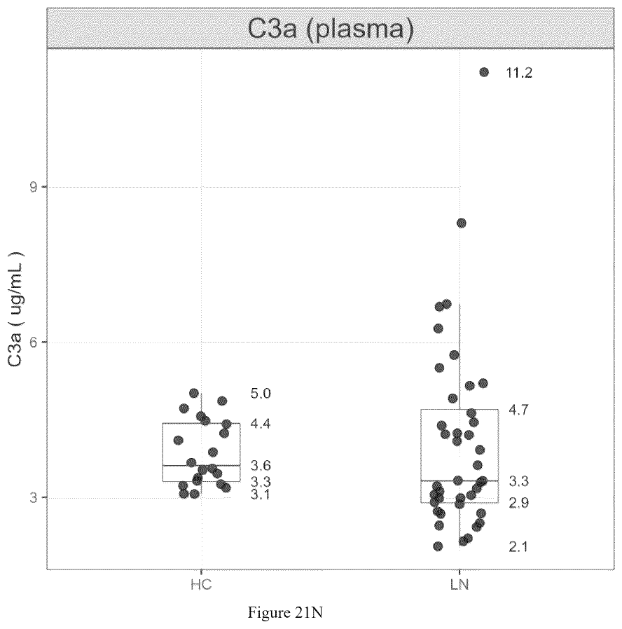

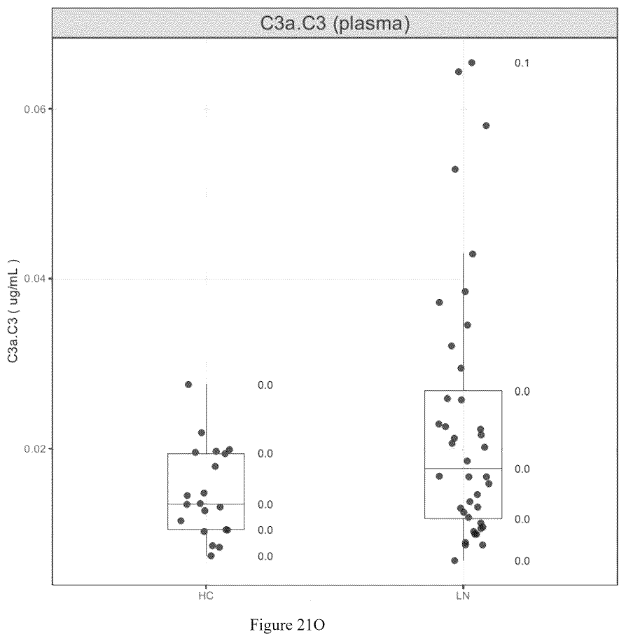

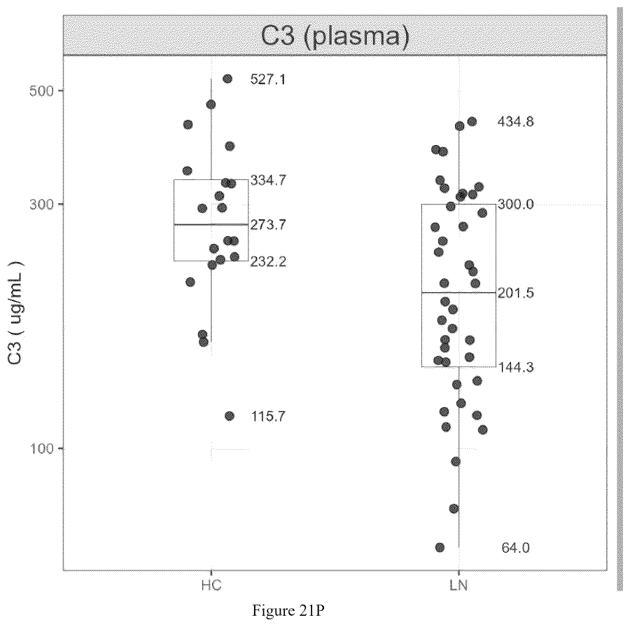

In addition, higher circulating levels of C4a, C4d, C1sC1 inhibitor, PACA1, PACA3 and C2b/C2 ratio, C1sC1 inhibitor/C1s ratio and C3a/C3 ratio, as well as reduced levels of C4, C2, C1s, and C3 in patients with active LN suggests complement may drive disease progression and severity. (FIGS. 21A-O).

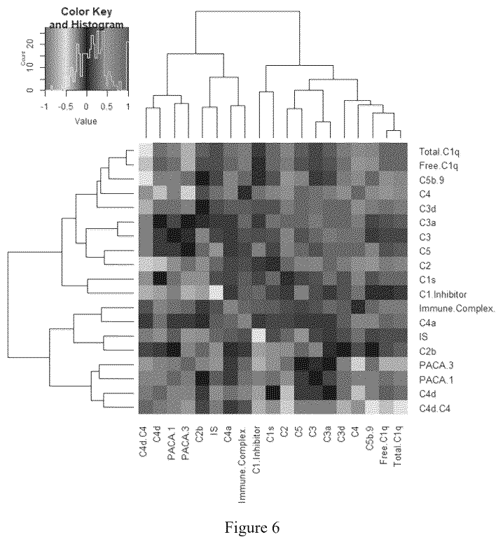

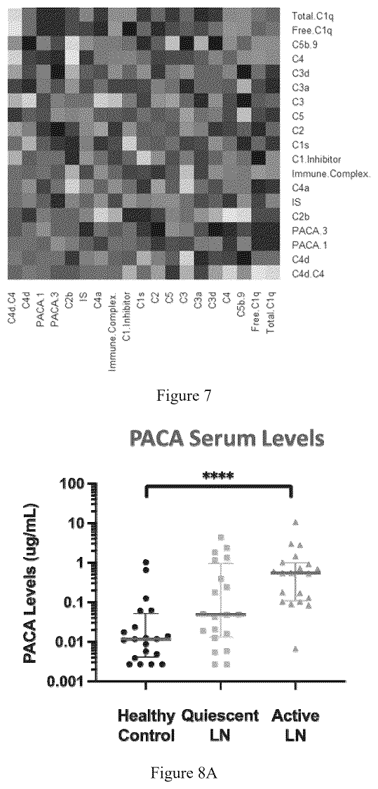

High correlation among complement factors in LN patients suggests coordinated pathway activity (FIG. 6 and FIG. 22). There was no correlation observed in Health controls (FIG. 7). The data shows strong association between high C4d/C4 ratio and presence of PACA. The data also shows strong association between other complement factors in LN patients such as, for example, C4d/C4 and C4d have strong positive correlation, C2b and C2b/C2 have a strong anti-correlation (FIG. 22). The correlation is used to select for patients most likely to respond to therapy with a classical complement pathway, and more specifically with an anti-C1q therapy.

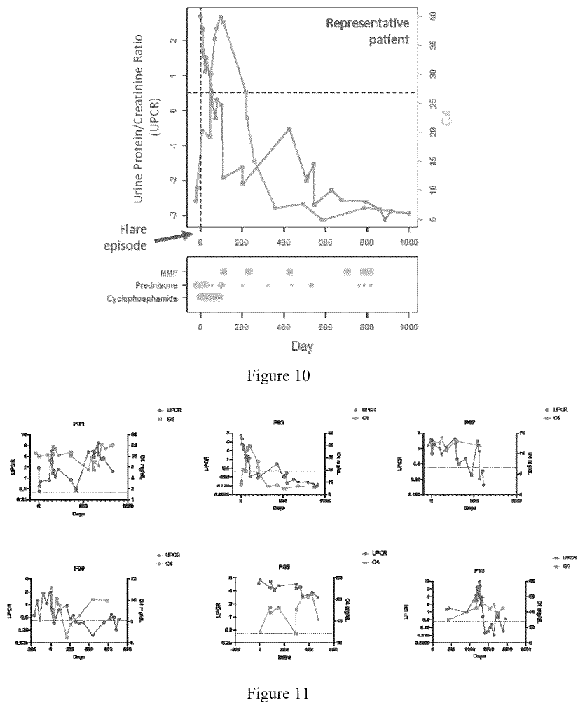

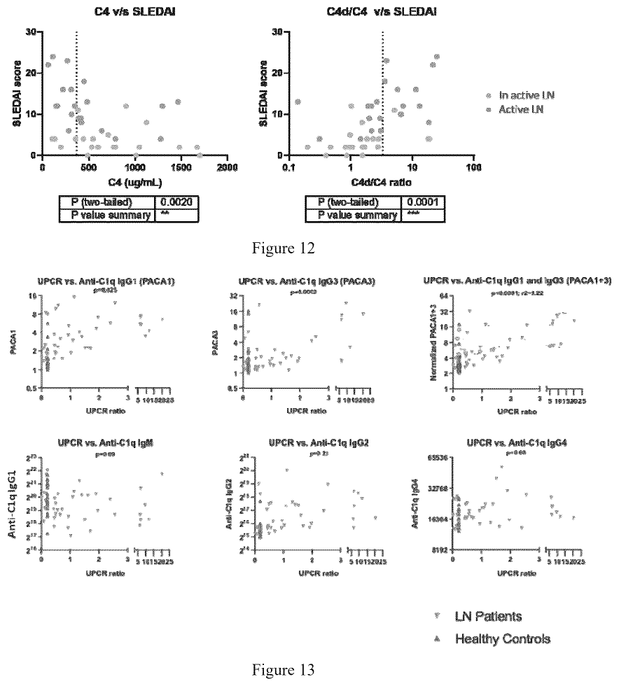

C4d/C4 is reduced post flare event by standard of care but effect is not durable (FIG. 9). FIG. 10 shows changes in urine protein/creatinine ratio (UPCR) and C4 in response to standard of care treatment. Rising level of C4 is approximately equivalent to reduction of C4d/C4 (C4d is novel, not routinely monitored). Rising level of C4 coincides with improved UPCR. Falling level of C4 post flare episode suggests residual inflammation that may lead to flare recurrence. The data also shows that clinical C4 measurements tracks UPCR trajectories (FIG. 11). Rise in C4 and decreased C4d/C4 correlates with improved disease activity (FIG. 12). Also low C4 and high C4d/C4 correlate with disease activity.

Presence of C4d on kidney biopsies indicates involvement of classical complement and unique specificity of C4d as biomarkers. Deposition of C4d in kidney biopsies suggests involvement of classical complement pathway in LN (FIG. 15). Further, high accuracy for this biomarker identifies SLE patients with LN (FIG. 16). High sensitivity and specificity of C4d/C4 as diagnostic biomarker over others (e.g., albuminuria, anti-DNA antibodies) suggests a potential causal role of classical complement in LN.

Plasma C4d can inform level of C4d in kidney tissue.

Together, this data provides strong scientific and clinical rationale for evaluating anti-C1q therapeutics in patients with Class III/IV LN who have evidence of classical complement pathway activation.

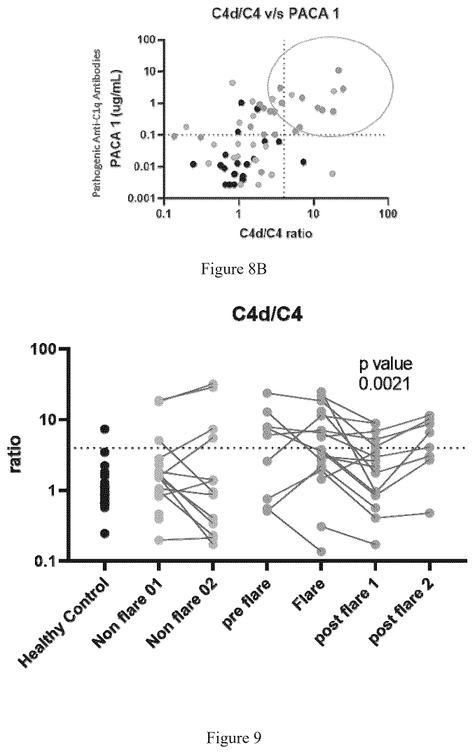

Active LN patients with high C4d/C4 ratio are likely to have higher PACA1 (FIGS. 8A-8B). PACA1 likely serves as the additive factor for amplifying disease severity. Improved C4d/C4 ratio post treatment further supports relevance of complement pathway in disease pathology (FIGS. 9 and 10).

PACA1 (Pathogenic Anti-C1q Antibody) levels remain elevated throughout treatment course. Active LN patients with high C4d/C4 ratio are likely to have higher PACA1 (FIGS. 8A-8B). PACA1 likely serves as the additive factor for amplifying disease severity. PACA (Pathogenic Anti-C1q Antibody) levels correlate with classical complement activation in lupus nephritis patients. Improved C4d/C4 ratio post treatment further supports relevance of complement pathway in disease pathology.

PACA's are distinct from therapeutic anti-C1q antibodies such as Mab2 and other therapeutic antibodies and antibody fragments that are designed to inhibit C1q function. Mab2 and other therapeutic antibodies and antibody fragments recognize the substrate-binding head groups of C1q and prevent C1q substrate binding/activation. In contrast, PACAs recognize the collagen tail of C1q when it is bound to substrate and recruit additional C1q via their Fc region-thereby enhancing C1q activity.

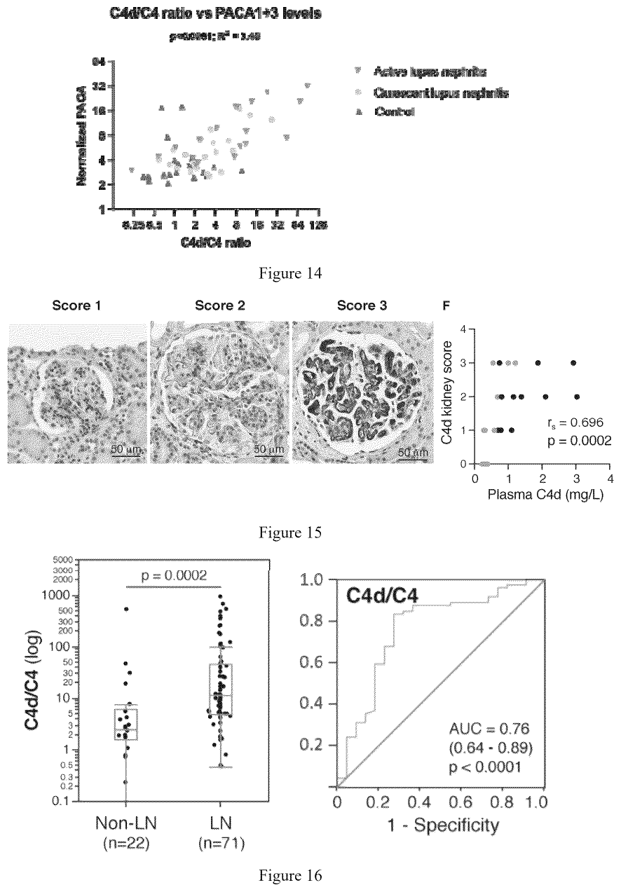

Described herein is the finding that PACA plasma titers correlate with the degree of kidney damage in lupus nephritis patients, as assessed by the urinary protein/creatinine ratio (UPCR) as a measure of proteinuria. In example 3 below, PACAs of the IgG1 (i.e., PACA1) and IgG3 isotypes (i.e. PACA3) provided the strongest degree of correlation with the UPCR, whereas there was no significant correlation with levels of IgG2 and IgG4 antibody against C1q (FIG. 13). These results are important because IgG1 and IgG3 are the antibody isotypes that most strongly recruit additional C1q (complement fixing isotypes), whereas IgG2 and IgG4 recruit little or no C1q to the immune complex, and hence are not associated with an increased level of disease. While IgM antibodies strongly activate C1q, they are characteristic of acute disease and would not be expected to circulate at appreciable levels in a chronic disorder such as lupus nephritis. PACA1 and PACA3 levels also correlate with C4d and the C4d/C4 ratio (FIG. 14).

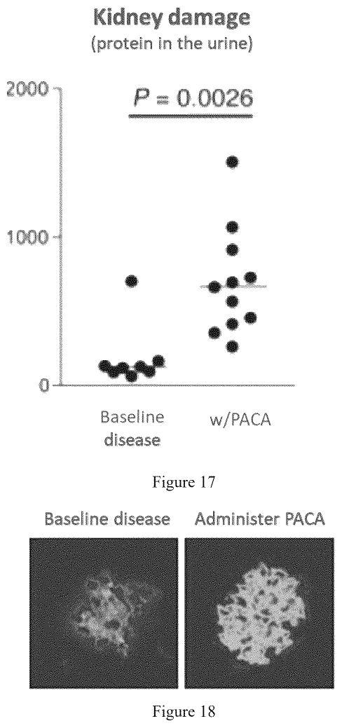

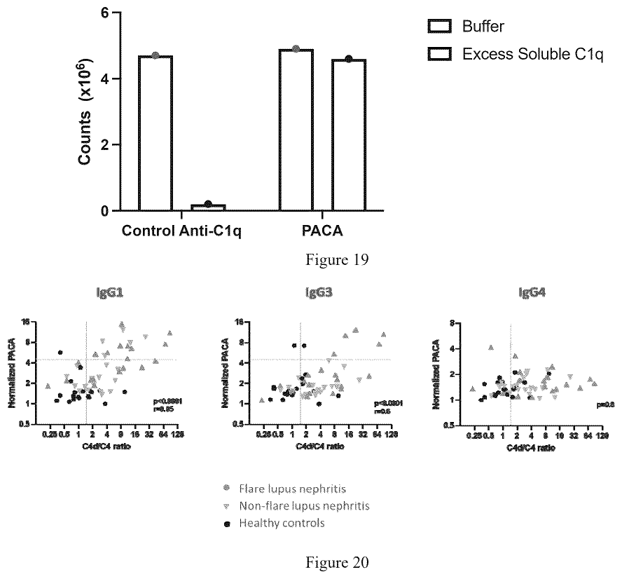

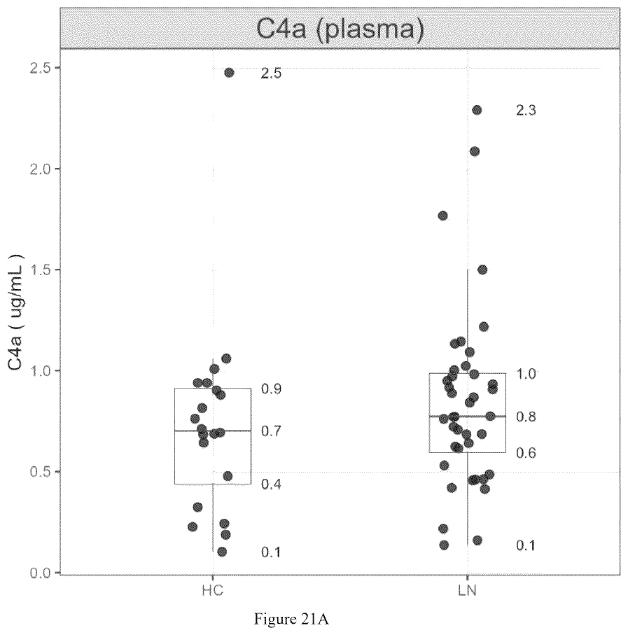

Moreover, early study with animal model of lupus nephritis reports causal link between anti-C1q antibodies and kidney damage. Administration of PACA greatly exacerbates disease with large increase in complement deposition in glomerular subendothelial space (FIGS. 17 and 18). PACAs are also characterized by ability to selectively bind substrate-bound C1q and activate classical complement pathway. PACA binding to substrate-bound C1q is not affected by soluble C1q (FIG. 19). Accordingly, PACA correlate with disease severity and are complement-activating IgG1 and IgG3 isotypes vs IgG4, which does not fix C1q. Anti-C1q IgG1 and IgG3, but not IgG4 levels correlate with serum C4d/C4 ratio (FIG. 20). This is consistent with complement activating capability of the antibody isotypes.

The present disclosure is generally directed to methods of treating lupus nephritis in a subject in need thereof. The method comprises determining that the subject has at least one of the following characteristics: an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. C4x may be C4a or C4d. The method further comprises administering to the subject an inhibitor of the classical complement pathway, e.g., if the subject has an elevated C4x level; an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; or an elevated Pathogenic Anti-C1q Antibody 1 (PACA1) and/or Pathogenic Anti-C1q Antibody 3 (PACA3) level. In some embodiments, the subject has at least two of the characteristics. For example, the characteristics may be selected from elevated C4x levels, elevated C4x/C4 ratio and reduced C4 levels, and/or the characteristics may be selected from the group consisting of elevated C1sC1 inhibitor levels, elevated C1sC1 inhibitor/C1s ratio and reduced C1s levels. In some embodiments, the subject further has an elevated Pathogenic Anti-C1q Antibody 1 (PACA1) and/or Pathogenic Anti-C1q Antibody 3 (PACA3) level. In some embodiments, the subject has at least one of the following additional characteristics: an elevated C3a level; an elevated C3a/C3 ratio; or a reduced C3 level. In some embodiments, the subject has elevated UPCR as yet another additional characteristic. A therapeutically effective amount of the inhibitor may be administered.

In some embodiments, the subject has an elevated C4x level. In some embodiments, the subject further has at least one of the following additional characteristics: an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or
a reduced C3 level. In some embodiments, the elevated C4x level is greater than a C4x level in normal or healthy subjects, is greater than a C4x level in normal or healthy subjects of a similar age, or is greater than a reference C4x level. The reference C4x level may be a value that is equal to or greater than the mean or median of C4x levels in samples derived from lupus nephritis subjects, or a value that is equal to or greater than the mean or median of C4x levels in samples derived from lupus nephritis subjects of a similar age. The reference C4x level may be a value that is equal to or greater than the 55th percentile, 60th percentile, 65th percentile, 70th percentile, 75th percentile, 80th percentile, 85th percentile, 90th percentile, 95th percentile, or 100th percentile of C4x levels in samples derived from lupus nephritis subjects. The reference C4x level may be a value that is equal to or greater than the 70th percentile of C4x levels in samples derived from lupus nephritis subjects. The reference C4x level may be a value that is equal to or greater than the 75th percentile of C4x levels in samples derived from lupus nephritis subjects. The reference C4x level may be a value that is equal to or greater than the 80th percentile of C4x levels in samples derived from lupus nephritis subjects. The reference C4x level may be a value that is equal to or greater than the 85th percentile of C4x levels in samples derived from lupus nephritis subjects. The reference C4x level may be a value that is equal to or greater than the 90th percentile of C4x levels in samples derived from lupus nephritis subjects. The reference C4x level may be a value that is equal to or greater than the 95th percentile of C4x levels in samples derived from lupus nephritis subjects. The reference C4x level may be a value that is equal to or greater than the 100th percentile of C4x levels in samples derived from lupus nephritis subjects. The reference C4x level may be a value that is equal to or greater than the 55th percentile, 60th percentile, 65th percentile, 70th percentile, 75th percentile, 80th percentile, 85th percentile, 90th percentile, 95th percentile, or 100th percentile of C4x levels in samples derived from lupus nephritis subjects of a similar age. The reference C4x level may be a value that is equal to or greater than the 70th percentile of C4x levels in samples derived from lupus nephritis subjects of a similar age. The reference C4x level may be a value that is equal to or greater than the 75th percentile of C4x levels in samples derived from lupus nephritis subjects of a similar age. The reference C4x level may be a value that is equal to or greater than the 80th percentile of C4x levels in samples derived from lupus nephritis subjects of a similar age. The reference C4x level may be a value that is equal to or greater than the 85th percentile of C4x levels in samples derived from lupus nephritis subjects of a similar age. The reference C4x level may be a value that is equal to or greater than the 90th percentile of C4x levels in samples derived from lupus nephritis subjects of a similar age. The reference C4x level may be a value that is equal to or greater than the 95th percentile of C4x levels in samples derived from lupus nephritis subjects of a similar age. The reference C4x level may be a value that is equal to or greater than the 100th percentile of C4x levels in samples derived from lupus nephritis subjects of a similar age. The reference C4x level may be a value that is equal to or greater than the 55th, 60th, 65th, 70th, 75th, 80th, 85th, 90th, 95th, or 100th percentile of C4x levels in samples derived from normal or healthy subjects. The reference C4x level may be a value that is equal to or greater than the 70th percentile of C4x levels in samples derived from normal or healthy subjects. The reference C4x level may be a value that is equal to or greater than the 75th percentile of C4x levels in samples derived from normal or healthy subjects. The reference C4x level may be a value that is equal to or greater than the 80th percentile of C4x levels in samples derived from normal or healthy subjects. The reference C4x level may be a value that is equal to or greater than the 85th percentile of C4x levels in samples derived from normal or healthy subjects. The reference C4x level may be a value that is equal to or greater than the 90th percentile of C4x levels in samples derived from normal or healthy subjects. The reference C4x level may be a value that is equal to or greater than the 95th percentile of C4x levels in samples derived from normal or healthy subjects. The reference C4x level may be a value that is equal to or greater than the 100th percentile of C4x levels in samples derived from normal or healthy subjects. The reference C4x level may be a value that is equal to or greater than the 55th, 60th, 65th, 70th, 75th, 80th, 85th, 90th, 95th, or 100th percentile of C4x levels in samples derived from normal or healthy subjects of a similar age. The reference C4x level may be a value that is equal to or greater than the 70th percentile of C4x levels in samples derived from normal or healthy subjects of a similar age. The reference C4x level may be a value that is equal to or greater than the 75th percentile of C4x levels in samples derived from normal or healthy subjects of a similar age. The reference C4x level may be a value that is equal to or greater than the 80th percentile of C4x levels in samples derived from normal or healthy subjects of a similar age. The reference C4x level may be a value that is equal to or greater than the 85th percentile of C4x levels in samples derived from normal or healthy subjects of a similar age. The reference C4x level may be a value that is equal to or greater than the 90th percentile of C4x levels in samples derived from normal or healthy subjects of a similar age. The reference C4x level may be a value that is equal to or greater than the 95th percentile of C4x levels in samples derived from normal or healthy subjects of a similar age. The reference C4x level may be a value that is equal to or greater than the 100th percentile of C4x levels in samples derived from normal or healthy subjects of a similar age. In some embodiments, the normal or healthy subjects do not have lupus nephritis. In some embodiments, the elevated C4x level is greater than the reference C4x level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%, or the elevated C4x level is greater than the reference C4x level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%

In some embodiments, the subject has an elevated C4x/C4 ratio. In some embodiments, the subject further has at least one of the following additional characteristics: an elevated C4x level; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C4x/C4 ratio is greater than a C4x/C4 ratio in normal or healthy subjects, is greater than a C4x/C4 ratio in normal or healthy subjects of a similar age, or is greater than a reference C4x/C4 ratio. The reference C4x/C4 ratio may be a value that is equal to or greater than the mean or median of C4x/C4 ratio in samples derived from lupus nephritis subjects, or may be equal to or greater than the mean or median of C4x/C4 ratio in samples derived from lupus nephritis subjects of a similar age. The reference C4x/C4 ratio may be a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects. The reference C4x/C4 ratio may be a value that is equal to or greater than the 70th percentile of C4x/C4 ratio in samples derived from lupus nephritis subjects. The reference C4x/C4 ratio may be a value that is equal to or greater than the 75th percentile of C4x/C4 ratio in samples derived from lupus nephritis subjects. The reference C4x/C4 ratio may be a value that is equal to or greater than the 80th percentile of C4x levels in samples derived from lupus nephritis subjects. The reference C4x/C4 ratio may be a value that is equal to or greater than the 85th percentile of C4x/C4 ratio in samples derived from lupus nephritis subjects. The reference C4x/C4 ratio may be a value that is equal to or greater than the 90th percentile of C4x/C4 ratio in samples derived from lupus nephritis subjects. The reference C4x/C4 ratio may be a value that is equal to or greater than the 95th percentile of C4x/C4 ratio in samples derived from lupus nephritis subjects. The reference C4x/C4 ratio may be a value that is equal to or greater than the 100th percentile of C4x/C4 ratio in samples derived from lupus nephritis subjects. The reference C4x/C4 ratio may be a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects of a similar age. The reference C4x/C4 ratio may be a value that is equal to or greater than the 70th percentile of C4x/C4 ratio in samples derived from lupus nephritis subjects of a similar age. The reference C4x/C4 ratio may be a value that is equal to or greater than the 75th percentile of C4x/C4 ratio in samples derived from lupus nephritis subjects of a similar age. The reference C4x/C4 ratio may be a value that is equal to or greater than the 80th percentile of C4x/C4 ratio in samples derived from lupus nephritis subjects of a similar age. The reference C4x/C4 ratio may be a value that is equal to or greater than the 85th percentile of C4x/C4 ratio in samples derived from lupus nephritis subjects of a similar age. The reference C4x/C4 ratio may be a value that is equal to or greater than the 90th percentile of C4x/C4 ratio in samples derived from lupus nephritis subjects of a similar age. The reference C4x/C4 ratio may be a value that is equal to or greater than the 95th percentile of C4x/C4 ratio in samples derived from lupus nephritis subjects of a similar age. The reference C4x/C4 ratio may be a value that is equal to or greater than the 100th percentile of C4x/C4 ratio in samples derived from lupus nephritis subjects of a similar age. The reference C4x/C4 ratio may be a value that is equal to or greater than the 55th, 60th, 65th, 70th, 75th, 80th, 85th, 90th, 95th, or 100th percentile of C4x/C4 ratio in samples derived from normal or healthy subjects. The reference C4x/C4 ratio may be a value that is equal to or greater than the 70th percentile of C4x/C4 ratio in samples derived from normal or healthy subjects. The reference C4x/C4 ratio may be a value that is equal to or greater than the 75th percentile of C4x/C4 ratio in samples derived from normal or healthy subjects. The reference C4x/C4 ratio may be a value that is equal to or greater than the 80th percentile of C4x/C4 ratio in samples derived from normal or healthy subjects. The reference C4x/C4 ratio may be a value that is equal to or greater than the 85th percentile of C4x/C4 ratio in samples derived from normal or healthy subjects. The reference C4x/C4 ratio may be a value that is equal to or greater than the 90th percentile of C4x/C4 ratio in samples derived from normal or healthy subjects. The reference C4x/C4 ratio may be a value that is equal to or greater than the 95th percentile of C4x/C4 ratio in samples derived from normal or healthy subjects. The reference C4x/C4 ratio may be a value that is equal to or greater than the 100th percentile of C4x/C4 ratio in samples derived from lupus normal or healthy. The reference C4x/C4 ratio may be a value that is equal to or greater than the 55th, 60th, 65th, 70th, 75th, 80th, 85th, 90th, 95th, or 100th percentile of C4x/C4 ratio in samples derived from normal or healthy subjects of a similar age. The reference C4x/C4 ratio may be a value that is equal to or greater than the 70th percentile of C4x/C4 ratio in samples derived from normal or healthy subjects of a similar age. The reference C4x/C4 ratio may be a value that is equal to or greater than the 75th percentile of C4x/C4 ratio in samples derived from normal or healthy subjects of a similar age. The reference C4x/C4 ratio may be a value that is equal to or greater than the 80th percentile of C4x/C4 ratio in samples derived from normal or healthy subjects of a similar age. The reference C4x/C4 ratio may be a value that is equal to or greater than the 85th percentile of C4x/C4 ratio in samples derived from normal or healthy subjects of a similar age. The reference C4x/C4 ratio may be a value that is equal to or greater than the 90th percentile of C4x/C4 ratio in samples derived from normal or healthy subjects of a similar age. The reference C4x/C4 ratio may be a value that is equal to or greater than the 95th percentile of C4x/C4 ratio in samples derived from normal or healthy subjects of a similar age. The reference C4x/C4 ratio may be a value that is equal to or greater than the 100th percentile of C4x/C4 ratio in samples derived from lupus normal or healthy of a similar age. In some embodiments, the normal or healthy subjects do not have lupus nephritis. In some embodiments, the elevated C4x/C4 ratio is greater than the reference C4x/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C4x/C4 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C4 level. In some embodiments, the subject further has at least one of the following additional characteristics: an elevated C4x level; an elevated C4x/C4 ratio; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C4 level is less than a C4 level in normal or healthy subjects. The reduced C4 level may be less than a C4 level in normal or healthy subjects of a similar age, may be less than a reference C4 level, or may be a value that is equal to or less than the mean or median of C4 levels in samples derived from lupus nephritis subjects. In some embodiments, the reference C4 level is a value that is equal to or less than the mean or median of C4 levels in samples derived from lupus nephritis subjects of a similar age. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects of a similar age. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects. The reference C4 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects of a similar age. In some embodiments, the normal or healthy subjects do not have lupus nephritis. In some embodiments, the reduced C4 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C1sC1 inhibitor level. In some embodiments, the subject has at least one of the following additional characteristics: an elevated C4x level; an elevated C4x/C4 ratio; a reduced C4 level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C1sC1 inhibitor level is greater than a C1sC1 inhibitor level in normal or healthy subjects, is greater than a CIsCI inhibitor level in normal or healthy subjects of a similar age, or is greater than a reference C1sC1 inhibitor level. The reference C1sC1 inhibitor level may be a value that is equal to or greater than the mean or median of C1sC1 inhibitor levels in samples derived from lupus nephritis subjects, or may be a value that is equal to or greater than the mean or median of C1sC1 inhibitor levels in samples derived from lupus nephritis subjects of a similar age. The reference C1sC1 inhibitor level may be a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects. The reference C1sC1 inhibitor level may be a value that is equal to or greater than the 70th percentile of C1sC1 inhibitor level in samples derived from lupus nephritis subjects. The reference C1sC1 inhibitor levels may be a value that is equal to or greater than the 75th percentile of C1sC1 inhibitor level in samples derived from lupus nephritis subjects. The reference C1sC1 inhibitor level may be a value that is equal to or greater than the 80th percentile of C1sC1 inhibitor level in samples derived from lupus nephritis subjects. The reference C1sC1 inhibitor level may be a value that is equal to or greater than the 85th percentile of C1sC1 inhibitor level in samples derived from lupus nephritis subjects. The reference C1sC1 inhibitor level may be a value that is equal to or greater than the 90th percentile of C1sC1 inhibitor level in samples derived from lupus nephritis subjects. The reference C1sC1 inhibitor level may be a value that is equal to or greater than the 95th percentile of C1sC1 inhibitor level in samples derived from lupus nephritis subjects. The reference C1sC1 inhibitor level may be a value that is equal to or greater than the 100th percentile of C1sC1 inhibitor level in samples derived from lupus nephritis subjects. The reference C1sC1 inhibitor level may be a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects of similar age. The reference C1sC1 inhibitor level may be a value that is equal to or greater than the 70th percentile of C1sC1 inhibitor level in samples derived from lupus nephritis subjects of similar age. The reference C1sC1 inhibitor levels may be a value that is equal to or greater than the 75th percentile of C1sC1 inhibitor level in samples derived from lupus nephritis subjects of similar age. The reference C1sC1 inhibitor level may be a value that is equal to or greater than the 80th percentile of C1sC1 inhibitor level in samples derived from lupus nephritis subjects of similar age. The reference C1sC1 inhibitor level may be a value that is equal to or greater than the 85th percentile of C1sC1 inhibitor level in samples derived from lupus nephritis subjects of similar age. The reference C1sC1 inhibitor level may be a value that is equal to or greater than the 90th percentile of C1sC1 inhibitor level in samples derived from lupus nephritis subjects of similar age. The reference C1sC1 inhibitor level may be a value that is equal to or greater than the 95th percentile of C1sC1 inhibitor level in samples derived from lupus nephritis subjects of similar age. The reference C1sC1 inhibitor level may be a value that is equal to or greater than the 100th percentile of C1sC1 inhibitor level in samples derived from lupus nephritis subjects of similar age. The reference C1sC1 inhibitor level may be a value that is equal to or greater than the 55th, 60th, 65th, 70th, 75th, 80th, 85th, 90th, 95th, or 100th percentile of C1sC1 inhibitor levels in samples derived from normal or healthy subjects. The reference C1sC1 inhibitor level may be a value that is equal to or greater than the 70th percentile of C1sC1 inhibitor level in samples derived from normal or healthy subjects. The reference C1sC1 inhibitor levels may be a value that is equal to or greater than the 75th percentile of C1sC1 inhibitor level in samples derived from normal or healthy subjects. The reference C1sC1 inhibitor level may be a value that is equal to or greater than the 80th percentile of C1sC1 inhibitor level in samples derived from normal or healthy subjects. The reference C1sC1 inhibitor level may be a value that is equal to or greater than the 85th percentile of C1sC1 inhibitor level in samples derived from normal or healthy subjects. The reference C1sC1 inhibitor level may be a value that is equal to or greater than the 90th percentile of C1sC1 inhibitor level in samples derived from normal or healthy subjects. The reference C1sC1 inhibitor level may be a value that is equal to or greater than the 95th percentile of C1sC1 inhibitor level in samples derived from normal or healthy subjects. The reference C1sC1 inhibitor level may be a value that is equal to or greater than the 100th percentile of C1sC1 inhibitor level in samples derived from normal or healthy. The reference C1sC1 inhibitor level may be a value that is equal to or greater than the 55th, 60th, 65th, 70th, 75th, 80th, 85th, 90th, 95th, or 100th percentile of C1sC1 inhibitor levels in samples derived from normal or healthy subjects of a similar age. The reference C1sC1 inhibitor level may be a value that is equal to or greater than the 70th percentile of C1sC1 inhibitor level in samples derived from normal or healthy subjects of a similar age. The reference C1sC1 inhibitor levels may be a value that is equal to or greater than the 75th percentile of C1sC1 inhibitor level in samples derived from normal or healthy subjects of a similar age. The reference C1sC1 inhibitor level may be a value that is equal to or greater than the 80th percentile of C1sC1 inhibitor level in samples derived from normal or healthy subjects of a similar age. The reference C1sC1 inhibitor level may be a value that is equal to or greater than the 85th percentile of C1sC1 inhibitor level in samples derived from normal or healthy subjects of a similar age. The reference C1sC1 inhibitor level may be a value that is equal to or greater than the 90th percentile of C1sC1 inhibitor level in samples derived from normal or healthy subjects of a similar age. The reference C1sC1 inhibitor level may be a value that is equal to or greater than the 95th percentile of C1sC1 inhibitor level in samples derived from normal or healthy subjects of a similar age. The reference C1sC1 inhibitor level may be a value that is equal to or greater than the 100th percentile of C1sC1 inhibitor level in samples derived from normal or healthy of a similar age. In some embodiments, the normal or healthy subjects do not have lupus nephritis. In some embodiments, the elevated C1sC1 inhibitor level is greater than the reference C1sC1 inhibitor level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C1sC1 inhibitor level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C1sC1 inhibitor/C1s ratio. In some embodiments, the subject has at least one of the following additional characteristics: an elevated C4x level; a reduced C4 level; an elevated C4x/C4 ratio; an elevated C1sC1 inhibitor level; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C1sC1 inhibitor/C1s ratio is greater than a C1sC1 inhibitor/C1s ratio in normal or healthy subjects. In some embodiments, the elevated C1sC1 inhibitor/C1s ratio is greater than a C1sC1 inhibitor/C1s ratio in normal or healthy subjects of a similar age. In some embodiments, the elevated C1sC1 inhibitor/C1s ratio is greater than a reference C1sC1 inhibitor/C1s ratio. In some embodiments, the reference C1sC1 inhibitor/C1s ratio is a value that is equal to or greater than the mean or median of C1sC1 inhibitor/C1s ratio in samples derived from lupus nephritis subjects. In some embodiments, the reference C1sC1 inhibitor/C1s ratio is a value that is equal to or greater than the mean or median of C1sC1 inhibitor/C1s ratio in samples derived from lupus nephritis subjects of a similar age. In some embodiments, the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/CIs ratio in samples derived from lupus nephritis subjects. The reference C1sC1 inhibitor/C1s ratio may be a value that is equal to or greater than the 70th percentile of C1sC1 inhibitor/C1s ratio in samples derived from lupus nephritis subjects. The reference C1sC1 inhibitor/C1s ratio may be a value that is equal to or greater than the 75th percentile of C1sC1 inhibitor/C1s ratio in samples derived from lupus nephritis subjects. The reference C1sC1 inhibitor/C1s ratio may be a value that is equal to or greater than the 80th percentile of C1sC1 inhibitor/C1s ratio in samples derived from lupus nephritis subjects. The reference C1sC1 inhibitor/C1s ratio may be a value that is equal to or greater than the 85th percentile of C1sC1 inhibitor/C1s ratio in samples derived from lupus nephritis subjects. The reference C1sC1 inhibitor/C1s ratio may be a value that is equal to or greater than the 90th percentile of C1sC1 inhibitor/C1s ratio in samples derived from lupus nephritis subjects. The reference C1sC1 inhibitor/C1s ratio may be a value that is equal to or greater than the 95th percentile of C1sC1 inhibitor/C1s ratio in samples derived from lupus nephritis subjects. The reference C1sC1 inhibitor/C1s ratio may be a value that is equal to or greater than the 100th percentile of C1sC1 inhibitor/C1s ratio in samples derived from lupus nephritis subjects. In some embodiments, the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from lupus nephritis subjects of a similar age. The reference C1sC1 inhibitor/C1s ratio may be a value that is equal to or greater than the 70th percentile of C1sC1 inhibitor/C1s ratio in samples derived from lupus nephritis subjects of a similar age. The reference C1sC1 inhibitor/C1s ratio may be a value that is equal to or greater than the 75th percentile of C1sC1 inhibitor/C1s ratio in samples derived from lupus nephritis subjects of a similar age. The reference C1sC1 inhibitor/C1s ratio may be a value that is equal to or greater than the 80th percentile of C1sC1 inhibitor/C1s in samples derived from lupus nephritis subjects of a similar age. The reference C1sC1 inhibitor/C1s ratio may be a value that is equal to or greater than the 85th percentile of C1sC1 inhibitor/C1s ratio in samples derived from lupus nephritis subjects of a similar age. The reference C1sC1 inhibitor/C1s ratio may be a value that is equal to or greater than the 90th percentile of C1sC1 inhibitor/C1s ratio in samples derived from lupus nephritis subjects of a similar age. The reference C1sC1 inhibitor/C1s ratio may be a value that is equal to or greater than the 95th percentile of C1sC1 inhibitor/C1s ratio in samples derived from lupus nephritis subjects of a similar age. The reference C1sC1 inhibitor/C1s ratio may be a value that is equal to or greater than the 100th percentile of C1sC1 inhibitor/C1s ratio in samples derived from lupus nephritis subjects of a similar age. In some embodiments, the reference C1sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from normal or healthy subjects. The reference C1sC1 inhibitor/C1s ratio may be a value that is equal to or greater than the 70th percentile of C1sC1 inhibitor/C1s ratio in samples derived from normal or healthy subjects. The reference C1sC1 inhibitor/C1s ratio may be a value that is equal to or greater than the 75th percentile of C1sC1 inhibitor/C1s ratio in samples derived from normal or healthy subjects. The reference C1sC1 inhibitor/C1s ratio may be a value that is equal to or greater than the 80th percentile of C1sC1 inhibitor/C1s in samples derived from normal or healthy subjects. The reference C1sC1 inhibitor/C1s ratio may be a value that is equal to or greater than the 85th percentile of C1sC1 inhibitor/C1s ratio in samples derived from normal or healthy subjects. The reference C1sC1 inhibitor/C1s ratio may be a value that is equal to or greater than the 90th percentile of C1sC1 inhibitor/C1s ratio in samples derived from normal or healthy subjects. The reference C1sC1 inhibitor/C1s ratio may be a value that is equal to or greater than the 95th percentile of C1sC1 inhibitor/C1s ratio in samples derived from normal or healthy subjects. The reference C1sC1 inhibitor/C1s ratio may be a value that is equal to or greater than the 100th percentile of C1sC1 inhibitor/C1s ratio in samples derived from normal or healthy subjects. In some embodiments, the reference C1sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from normal or healthy subjects of a similar age. The reference C1sC1 inhibitor/C1s ratio may be a value that is equal to or greater than the 70th percentile of C1sC1 inhibitor/C1s ratio in samples derived from normal or healthy subjects of a similar age. The reference C1sC1 inhibitor/C1s ratio may be a value that is equal to or greater than the 75th percentile of C1sC1 inhibitor/C1s ratio in samples derived from normal or healthy subjects of a similar age. The reference C1sC1 inhibitor/C1s ratio may be a value that is equal to or greater than the 80th percentile of C1sC1 inhibitor/C1s in samples derived from normal or healthy subjects of a similar age. The reference C1sC1 inhibitor/C1s ratio may be a value that is equal to or greater than the 85th percentile of C1sC1 inhibitor/C1s ratio in samples derived from normal or healthy subjects of a similar age. The reference C1sC1 inhibitor/C1s ratio may be a value that is equal to or greater than the 90th percentile of C1sC1 inhibitor/C1s ratio in samples derived from normal or healthy subjects of a similar age. The reference C1sC1 inhibitor/C1s ratio may be a value that is equal to or greater than the 95th percentile of C1sC1 inhibitor/C1s ratio in samples derived from normal or healthy subjects of a similar age. The reference C1sC1 inhibitor/C1s ratio may be a value that is equal to or greater than the 100th percentile of C1sC1 inhibitor/C1s ratio in samples derived from normal or healthy subjects of a similar age. In some embodiments, the normal or healthy subjects do not have lupus nephritis. In some embodiments, the elevated C1sC1 inhibitor/C1s ratio is greater than the reference C1sC1 inhibitor/C1s ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C1sC1 inhibitor/C1s ratio is greater than the reference C1sC1 inhibitor/C1s ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C1s level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C1s level is less than a C1s level in normal or healthy subjects. In some embodiments, the reduced C1s level is less than a C1s level in normal or healthy subjects of a similar age. In some embodiments, the reduced C1s level is less than a reference C1s level. In some embodiments, the reference C1s level is a value that is equal to or less than the mean or median of C1s levels in samples derived from lupus nephritis subjects. In some embodiments, the reference C1s level is a value that is equal to or less than the mean or median of C1s levels in samples derived from lupus nephritis subjects of a similar age. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s level in samples derived from lupus nephritis subjects. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s level in samples derived from lupus nephritis subjects of a similar age. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects. In some embodiments, the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects of a similar age. In some embodiments, the normal or healthy subjects do not have lupus nephritis. In some embodiments, the reduced C1s level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the reduced C1s level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C2b level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; a reduced C1s level, an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C2b level is greater than a C2b level in normal or healthy subjects, the elevated C2b level is greater than a C2b level in normal or healthy subjects of a similar age, or is greater than a reference C2b level. The reference C2b level may be a value that is equal to or greater than the mean or median of C2b levels in samples derived from lupus nephritis subjects, or is a value that is equal to or greater than the mean or median of C2b levels in samples derived from lupus nephritis subjects of a similar age. The reference C2b level may be a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects. The reference C2b level may be a value that is equal to or greater than the 70th percentile of C2b level in samples derived from lupus nephritis subjects. The reference C2b level may be a value that is equal to or greater than the 75th percentile of C2b level in samples derived from lupus nephritis subjects. The reference C2b level may be a value that is equal to or greater than the 80th percentile of C2b level in samples derived from lupus nephritis subjects. The reference C2b level may be a value that is equal to or greater than the 85th percentile of C2b level in samples derived from lupus nephritis subjects. The reference C2b level may be a value that is equal to or greater than the 90th percentile of C2b level in samples derived from lupus nephritis subjects. The reference C2b level may be a value that is equal to or greater than the 95th percentile of C2b level in samples derived from lupus nephritis subjects. The reference C2b level may be a value that is equal to or greater than the 100th percentile of C2b level in samples derived from lupus nephritis subjects. The reference C2b level may be a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects of a similar age. The reference C2b level may be a value that is equal to or greater than the 70th percentile of C2b level in samples derived from lupus nephritis subjects of a similar age. The reference C2b level may be a value that is equal to or greater than the 75th percentile of C2b level in samples derived from lupus nephritis subjects of a similar age. The reference C2b level may be a value that is equal to or greater than the 80th percentile of C2b level in samples derived from lupus nephritis subjects of a similar age. The reference C2b level may be a value that is equal to or greater than the 85th percentile of C2b level in samples derived from lupus nephritis subjects of a similar age. The reference C2b level may be a value that is equal to or greater than the 90th percentile of C2b level in samples derived from lupus nephritis subjects of a similar age. The reference C2b level may be a value that is equal to or greater than the 95th percentile of C2b level in samples derived from lupus nephritis subjects of a similar age. The reference C2b level may be a value that is equal to or greater than the 100th percentile of C2b level in samples derived from lupus nephritis subjects of a similar age. The reference C2b level may be a value that is equal to or greater than the 55th, 60th, 65th, 70th, 75th, 80th, 85th, 90th, 95th, or 100th percentile of C2b levels in samples derived from normal or healthy subjects. The reference C2b level may be a value that is equal to or greater than the 70th percentile of C2b level in samples derived from normal or healthy subjects. The reference C2b level may be a value that is equal to or greater than the 75th percentile of C2b level in samples derived from normal or healthy subjects. The reference C2b level may be a value that is equal to or greater than the 80th percentile of C2b level in samples derived from normal or healthy subjects. The reference C2b level may be a value that is equal to or greater than the 85th percentile of C2b level in samples derived from normal or healthy subjects. The reference C2b level may be a value that is equal to or greater than the 90th percentile of C2b level in samples derived from normal or healthy subjects. The reference C2b level may be a value that is equal to or greater than the 95th percentile of C2b level in samples derived from normal or healthy subjects. The reference C2b level may be a value that is equal to or greater than the 100th percentile of C2b level in samples derived from normal or healthy subjects. The reference C2b level may be a value that is equal to or greater than the 55th, 60th, 65th, 70th, 75th, 80th, 85th, 90th, 95th, or 100th percentile of C2b levels in samples derived from normal or healthy subjects of a similar age. The reference C2b level may be a value that is equal to or greater than the 70th percentile of C2b level in samples derived from normal or healthy subjects of a similar age. The reference C2b level may be a value that is equal to or greater than the 75th percentile of C2b level in samples derived from normal or healthy subjects of a similar age. The reference C2b level may be a value that is equal to or greater than the 80th percentile of C2b level in samples derived from normal or healthy subjects of a similar age. The reference C2b level may be a value that is equal to or greater than the 85th percentile of C2b level in samples derived from normal or healthy subjects of a similar age. The reference C2b level may be a value that is equal to or greater than the 90th percentile of C2b level in samples derived from normal or healthy subjects of a similar age. The reference C2b level may be a value that is equal to or greater than the 95th percentile of C2b level in samples derived from normal or healthy subjects of a similar age. The reference C2b level may be a value that is equal to or greater than the 100th percentile of C2b level in samples derived from normal or healthy subjects of a similar age. In some embodiments, the normal or healthy subjects do not have lupus nephritis. In some embodiments, the elevated C2b level is greater than the reference C2b level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference C2b level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C2b/C2 ratio. In some embodiments, the subject has at least one of the following additional characteristics: an elevated C4x level; an elevated C4x/C4 ratio; reduced C4 level; an elevated C1sC1 inhibitor/C1s ratio; an elevated C1sC1 inhibitor level; a reduced C1s level; a reduced C2 level; an elevated C2b level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the elevated C2b/C2 ratio is greater than a C2b/C2 ratio in normal or healthy subjects. In some embodiments, the elevated C2b/C2 ratio is greater than a C2b/C2 ratio in normal or healthy subjects of a similar age. In some embodiments, the elevated C2b/C2 ratio is greater than a reference C2b/C2 ratio. In some embodiments, the reference C2b/C2 ratio is a value that is equal to or greater than the mean or median of C2b/C2 ratio in samples derived from lupus nephritis subjects. In some embodiments, the reference C2b/C2 ratio is a value that is equal to or greater than the mean or median of C2b/C2 ratio in samples derived from lupus nephritis subjects of a similar age. In some embodiments, the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects. In some embodiments, the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects of a similar age. In some embodiments, the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects. In some embodiments, the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects of a similar age. In some embodiments, the normal or healthy subjects do not have lupus nephritis. In some embodiments, the elevated C2b/C2 ratio is greater than the reference C2b/C2 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C2b/C2 ratio is greater than the reference C2b/C2 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C2 level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; a reduced C1s level; an elevated C2b level; an elevated C2b/C2 ratio; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C3 level. In some embodiments, the reduced C2 level is less than a C2 level in normal or healthy subjects, is less than a C2 level in normal or healthy subjects of a similar age, or is less than a reference C2 level. The reference C2 level may be a value that is equal to or less than the mean or median of C2 levels in samples derived from lupus nephritis subjects or may be a value that is equal to or less than the mean or median of C2 levels in samples derived from lupus nephritis subjects of a similar age. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects of a similar age. The reference C2 level may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects or may be a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects of a similar age. In some embodiments, the normal or healthy subjects do not have lupus nephritis. In some embodiments, the reduced C2 level is less than the reference C2 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is less than the reference C2 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated Pathogenic Anti-C1q Antibody 1 (PACA1) and/or Pathogenic Anti-C1q Antibody 3 (PACA3) level. In some embodiments, the subject has at least one of the following additional characteristics: an elevated C4x level; an elevated C4x/C4 ratio; a reduced C4 level; an elevated C1sC1 inhibitor/C1s ratio; an elevated C1sC1 inhibitor level; a reduced C1s level; an elevated C2b level; an elevated C2b/C2 ratio; a reduced C2 level; an elevated C3a level; an elevated C3a/C3 ratio; or a reduced C3 level. In some embodiments, the elevated PACA1 and/or PACA3 level is greater than a PACA1 and/or PACA3 level in normal or healthy subjects, is greater than a PACA1 and/or PACA3 level in normal or healthy subjects of a similar age, or is greater than a reference PACA1 and/or PACA3 level. The reference PACA1 and/or PACA3 level may be a value that is equal to or greater than the mean or median of PACA1 and/or PACA3 levels in samples derived from lupus nephritis subjects or may be a value that is equal to or greater than the mean or median of PACA1 and/or PACA3 levels in samples derived from lupus nephritis subjects of a similar age. The reference PACA1 and/or PACA3 level may be a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects. The reference PACA1 and/or PACA3 level may be a value that is equal to or greater than the 70th percentile of PACA1 and/or PACA3 level in samples derived from lupus nephritis subjects. The reference PACA1 and/or PACA3 level may be a value that is equal to or greater than the 75th percentile of PACA1 and/or PACA3 level in samples derived from lupus nephritis subjects. The reference PACA1 and/or PACA3 level may be a value that is equal to or greater than the 80th percentile of PACA1 and/or PACA3 level in samples derived from lupus nephritis subjects. The reference PACA1 and/or PACA3 level may be a value that is equal to or greater than the 85th percentile of PACA1 and/or PACA3 level in samples derived from lupus nephritis subjects. The reference PACA1 and/or PACA3 level may be a value that is equal to or greater than the 90th percentile of PACA1 and/or PACA3 level in samples derived from lupus nephritis subjects. The reference PACA1 and/or PACA3 level may be a value that is equal to or greater than the 95th percentile of PACA1 and/or PACA3 level in samples derived from lupus nephritis subjects. The reference PACA1 and/or PACA3 level may be a value that is equal to or greater than the 100th percentile of PACA1 and/or PACA3 level in samples derived from lupus nephritis subjects. The reference PACA1 and/or PACA3 level may be a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects of a similar age. The reference PACA1 and/or PACA3 level may be a value that is equal to or greater than the 70th percentile of PACA1 and/or PACA3 level in samples derived from lupus nephritis subjects of a similar age. The reference PACA1 and/or PACA3 level may be a value that is equal to or greater than the 75th percentile of PACA1 and/or PACA3 level in samples derived from lupus nephritis subjects of a similar age. The reference PACA1 and/or PACA3 level may be a value that is equal to or greater than the 80th percentile of PACA1 and/or PACA3 level in samples derived from lupus nephritis subjects of a similar age. The reference PACA1 and/or PACA3 level may be a value that is equal to or greater than the 85th percentile of PACA1 and/or PACA3 level in samples derived from lupus nephritis subjects of a similar age. The reference PACA1 and/or PACA3 level may be a value that is equal to or greater than the 90th percentile of PACA1 and/or PACA3 level in samples derived from lupus nephritis subjects of a similar age. The reference PACA1 and/or PACA3 level may be a value that is equal to or greater than the 95th percentile of PACA1 and/or PACA3 level in samples derived from lupus nephritis subjects of a similar age. The reference PACA1 and/or PACA3 level may be a value that is equal to or greater than the 100th percentile of PACA1 and/or PACA3 level in samples derived from lupus nephritis subjects of a similar age. The reference PACA1 and/or PACA3 level may be a value that is equal to or greater than the 55th, 60th, 65th, 70th, 75th, 80th, 85th, 90th, 95th, or 100th percentile of PACA1 and/or PACA3 levels in samples derived from normal or healthy subjects. The reference PACA1 and/or PACA3 level may be a value that is equal to or greater than the 70th percentile of PACA1 and/or PACA3 level in samples derived from normal or healthy subjects. The reference PACA1 and/or PACA3 level may be a value that is equal to or greater than the 75th percentile of PACA1 and/or PACA3 level in samples derived from normal or healthy subjects. The reference PACA1 and/or PACA3 level may be a value that is equal to or greater than the 80th percentile of PACA1 and/or PACA3 level in samples derived from normal or healthy subjects. The reference PACA1 and/or PACA3 level may be a value that is equal to or greater than the 85th percentile of PACA1 and/or PACA3 level in samples derived from normal or healthy subjects. The reference PACA1 and/or PACA3 level may be a value that is equal to or greater than the 90th percentile of PACA1 and/or PACA3 level in samples derived from normal or healthy subjects. The reference PACA1 and/or PACA3 level may be a value that is equal to or greater than the 95th percentile of PACA1 and/or PACA3 level in samples derived from normal or healthy subjects. The reference PACA1 and/or PACA3 level may be a value that is equal to or greater than the 100th percentile of PACA1 and/or PACA3 level in samples derived from normal or healthy subjects. The reference PACA1 and/or PACA3 level may be a value that is equal to or greater than the 55th, 60th, 65th, 70th, 75th, 80th, 85th, 90th, 95th, or 100th percentile of PACA1 and/or PACA3 levels in samples derived from normal or healthy subjects of a similar age. The reference PACA1 and/or PACA3 level may be a value that is equal to or greater than the 70th percentile of PACA1 and/or PACA3 level in samples derived from normal or healthy subjects of a similar age. The reference PACA1 and/or PACA3 level may be a value that is equal to or greater than the 75th percentile of PACA1 and/or PACA3 level in samples derived from normal or healthy subjects of a similar age. The reference PACA1 and/or PACA3 level may be a value that is equal to or greater than the 80th percentile of PACA1 and/or PACA3 level in samples derived from normal or healthy subjects of a similar age. The reference PACA1 and/or PACA3 level may be a value that is equal to or greater than the 85th percentile of PACA1 and/or PACA3 level in samples derived from normal or healthy subjects of a similar age. The reference PACA1 and/or PACA3 level may be a value that is equal to or greater than the 90th percentile of PACA1 and/or PACA3 level in samples derived from normal or healthy subjects of a similar age. The reference PACA1 and/or PACA3 level may be a value that is equal to or greater than the 95th percentile of PACA1 and/or PACA3 level in samples derived from normal or healthy subjects of a similar age. The reference PACA1 and/or PACA3 level may be a value that is equal to or greater than the 100th percentile of PACA1 and/or PACA3 level in samples derived from normal or healthy subjects of a similar age. In some embodiments, the normal or healthy subjects do not have lupus nephritis. In some embodiments, the elevated PACA1 and/or PACA3 level is greater than the reference PACA1 and/or PACA3 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500% or is greater than the reference PACA1 and/or PACA3 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C3a level. In some embodiments, the subject has at least one of the following additional characteristics: an elevated C4x/C4 ratio; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C4x level; an elevated C3a/C3 ratio; or a reduced C3 level. In some embodiments, the elevated C3a level is greater than a C3a level in normal or healthy subjects. In some embodiments, the elevated C3a level is greater than a C3a level in normal or healthy subjects of a similar age. In some embodiments, the elevated C3a level is greater than a reference C3a level. In some embodiments, the reference C3a level is a value that is equal to or greater than the mean or median of C3a levels in samples derived from lupus nephritis subjects. In some embodiments, the reference C3a level is a value that is equal to or greater than the mean or median of C3a levels in samples derived from lupus nephritis subjects of a similar age. In some embodiments, the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects. The reference C3a level may be a value that is equal to or greater than the 70th percentile of C3a level in samples derived from lupus nephritis subjects. The reference C3a level may be a value that is equal to or greater than the 75th percentile of C3a level in samples derived from lupus nephritis subjects. The reference C3a level may be a value that is equal to or greater than the 80th percentile of C3a level in samples derived from lupus nephritis subjects. The reference C3a level may be a value that is equal to or greater than the 85th percentile of C3a level in samples derived from lupus nephritis subjects. The reference C3a level may be a value that is equal to or greater than the 90th percentile of C3a level in samples derived from lupus nephritis subjects. The reference C3a level may be a value that is equal to or greater than the 95th percentile of C3a level in samples derived from lupus nephritis subjects. The reference C3a level may be a value that is equal to or greater than the 100th percentile of C3a level in samples derived from lupus nephritis subjects. In some embodiments, the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects of a similar age. The reference C3a level may be a value that is equal to or greater than the 70th percentile of C3a level in samples derived from lupus nephritis subjects of a similar age. The reference C3a level may be a value that is equal to or greater than the 75th percentile of C3a level in samples derived from lupus nephritis subjects of a similar age. The reference C3a level may be a value that is equal to or greater than the 80th percentile of C3a level in samples derived from lupus nephritis subjects of a similar age. The reference C3a level may be a value that is equal to or greater than the 85th percentile of C3a level in samples derived from lupus nephritis subjects of a similar age. The reference C3a level may be a value that is equal to or greater than the 90th percentile of C3a level in samples derived from lupus nephritis subjects of a similar age. The reference C3a level may be a value that is equal to or greater than the 95th percentile of C3a level in samples derived from lupus nephritis subjects of a similar age. The reference C3a level may be a value that is equal to or greater than the 100th percentile of C3a level in samples derived from lupus nephritis subjects of a similar age. In some embodiments, the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects. The reference C3a level may be a value that is equal to or greater than the 70th percentile of C3a level in samples derived from normal or healthy subjects. The reference C3a level may be a value that is equal to or greater than the 75th percentile of C3a level in samples derived from normal or healthy subjects. The reference C3a level may be a value that is equal to or greater than the 80th percentile of C3a level in samples derived from normal or healthy subjects. The reference C3a level may be a value that is equal to or greater than the 85th percentile of C3a level in samples derived from normal or healthy subjects. The reference C3a level may be a value that is equal to or greater than the 90th percentile of C3a level in samples derived from normal or healthy subjects. The reference C3a level may be a value that is equal to or greater than the 95th percentile of C3a level in samples derived from normal or healthy subjects. The reference C3a level may be a value that is equal to or greater than the 100th percentile of C3a level in samples derived from normal or healthy subjects. In some embodiments, the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects of a similar age. The reference C3a level may be a value that is equal to or greater than the 70th percentile of C3a level in samples derived from normal or healthy subjects of a similar age. The reference C3a level may be a value that is equal to or greater than the 75th percentile of C3a level in samples derived from normal or healthy subjects of a similar age. The reference C3a level may be a value that is equal to or greater than the 80th percentile of C3a level in samples derived from normal or healthy subjects of a similar age. The reference C3a level may be a value that is equal to or greater than the 85th percentile of C3a level in samples derived from normal or healthy subjects of a similar age. The reference C3a level may be a value that is equal to or greater than the 90th percentile of C3a level in samples derived from normal or healthy subjects of a similar age. The reference C3a level may be a value that is equal to or greater than the 95th percentile of C3a level in samples derived from normal or healthy subjects of a similar age. The reference C3a level may be a value that is equal to or greater than the 100th percentile of C3a level in samples derived from normal or healthy subjects of a similar age. In some embodiments, the normal or healthy subjects do not have lupus nephritis. In some embodiments, the elevated C3a level is greater than the reference C3a level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C3a level is greater than the reference C3a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has an elevated C3a/C3 ratio. In some embodiments, the subject has at least one of the following additional characteristics: an elevated C4x level; a reduced C4 level; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C4x/C4 ratio; or a reduced C3 level. In some embodiments, the elevated C3a/C3 ratio is greater than a C3a/C3 ratio in normal or healthy subjects. In some embodiments, the elevated C3a/C3 ratio is greater than a C3a/C3 ratio in normal or healthy subjects of a similar age. In some embodiments, the elevated C3a/C3 ratio is greater than a reference C3a/C3 ratio. In some embodiments, the reference C3a/C3 ratio is a value that is equal to or greater than the mean or median of C3a/C3 ratio in samples derived from lupus nephritis subjects. In some embodiments, the reference C3a/C3 ratio is a value that is equal to or greater than the mean or median of C3a/C3 ratio in samples derived from lupus nephritis subjects of a similar age. In some embodiments, the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects. The reference C3a/C3 ratio may be a value that is equal to or greater than the 70th percentile of C3a/C3 ratio in samples derived from lupus nephritis subjects. The reference C3a/C3 ratio may be a value that is equal to or greater than the 75th percentile of C3a/C3 ratio in samples derived from lupus nephritis subjects. The reference C3a/C3 ratio may be a value that is equal to or greater than the 80th percentile of C3a/C3 ratio in samples derived from lupus nephritis subjects. The reference C3a/C3 ratio may be a value that is equal to or greater than the 85th percentile of C3a/C3 ratio in samples derived from lupus nephritis subjects. The reference C3a/C3 ratio may be a value that is equal to or greater than the 90th percentile of C3a/C3 ratio in samples derived from lupus nephritis subjects. The reference C3a/C3 ratio may be a value that is equal to or greater than the 95th percentile of C3a/C3 ratio in samples derived from lupus nephritis subjects. The reference C3a/C3 ratio may be a value that is equal to or greater than the 100th percentile of C3a/C3 ratio in samples derived from lupus nephritis subjects. In some embodiments, the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects of a similar age. The reference C3a/C3 ratio may be a value that is equal to or greater than the 70th percentile of C3a/C3 ratio in samples derived from lupus nephritis subjects of a similar age. The reference C3a/C3 ratio may be a value that is equal to or greater than the 75th percentile of C3a/C3 ratio in samples derived from lupus nephritis subjects of a similar age. The reference C3a/C3 ratio may be a value that is equal to or greater than the 80th percentile of C3a/C3 ratio in samples derived from lupus nephritis subjects of a similar age. The reference C3a/C3 ratio may be a value that is equal to or greater than the 85th percentile of C3a/C3 ratio in samples derived from lupus nephritis subjects of a similar age. The reference C3a/C3 ratio may be a value that is equal to or greater than the 90th percentile of C3a/C3 ratio in samples derived from lupus nephritis subjects of a similar age. The reference C3a/C3 ratio may be a value that is equal to or greater than the 95th percentile of C3a/C3 ratio in samples derived from lupus nephritis subjects of a similar age. The reference C3a/C3 ratio may be a value that is equal to or greater than the 100th percentile of C3a/C3 ratio in samples derived from lupus nephritis subjects of a similar age. In some embodiments, the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects. The reference C3a/C3 ratio may be a value that is equal to or greater than the 70th percentile of C3a/C3 ratio in samples derived from normal or healthy subjects. The reference C3a/C3 ratio may be a value that is equal to or greater than the 75th percentile of C3a/C3 ratio in samples derived from normal or healthy subjects. The reference C3a/C3 ratio may be a value that is equal to or greater than the 80th percentile of C3a/C3 ratio in samples derived from normal or healthy subjects. The reference C3a/C3 ratio may be a value that is equal to or greater than the 85th percentile of C3a/C3 ratio in samples derived from normal or healthy subjects. The reference C3a/C3 ratio may be a value that is equal to or greater than the 90th percentile of C3a/C3 ratio in samples derived from normal or healthy subjects. The reference C3a/C3 ratio may be a value that is equal to or greater than the 95th percentile of C3a/C3 ratio in samples derived from normal or healthy subjects. The reference C3a/C3 ratio may be a value that is equal to or greater than the 100th percentile of C3a/C3 ratio in samples derived from normal or healthy subjects. In some embodiments, the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects of a similar age. The reference C3a/C3 ratio may be a value that is equal to or greater than the 70th percentile of C3a/C3 ratio in samples derived from normal or healthy subjects of a similar age. The reference C3a/C3 ratio may be a value that is equal to or greater than the 75th percentile of C3a/C3 ratio in samples derived from normal or healthy subjects of a similar age. The reference C3a/C3 ratio may be a value that is equal to or greater than the 80th percentile of C3a/C3 ratio in samples derived from normal or healthy subjects of a similar age. The reference C3a/C3 ratio may be a value that is equal to or greater than the 85th percentile of C3a/C3 ratio in samples derived from normal or healthy subjects of a similar age. The reference C3a/C3 ratio may be a value that is equal to or greater than the 90th percentile of C3a/C3 ratio in samples derived from normal or healthy subjects of a similar age. The reference C3a/C3 ratio may be a value that is equal to or greater than the 95th percentile of C3a/C3 ratio in samples derived from normal or healthy subjects of a similar age. The reference C3a/C3 ratio may be a value that is equal to or greater than the 100th percentile of C3a/C3 ratio in samples derived from normal or healthy subjects of a similar age. In some embodiments, the normal or healthy subjects do not have lupus nephritis. In some embodiments, the elevated C3a/C3 ratio is greater than the reference C3a/C3 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the elevated C3a/C3 ratio is greater than the reference C3a/C3 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the subject has a reduced C3 level. In some embodiments, the subject has at least one of the following additional characteristics: an elevated C4x level; an elevated C4x/C4 ratio; an elevated C1sC1 inhibitor level; an elevated C1sC1 inhibitor/C1s ratio; a reduced C1s level; an elevated C2b level; an elevated C2b/C2 ratio; a reduced C2 level; an elevated Pathogenic Anti-C1q Antibody 1 (PACA1) and/or Pathogenic Anti-C1q Antibody 3 (PACA3) level; an elevated C3a level; an elevated C3a/C3 ratio; or a reduced C4 level. In some embodiments, the reduced C3 level is less than a C3 level in normal or healthy subjects. In some embodiments, the reduced C3 level is less than a C3 level in normal or healthy subjects of a similar age. In some embodiments, the reduced C3 level is less than a reference C3 level. In some embodiments, the reference C3 level is a value that is equal to or less than the mean or median of C3 levels in samples derived from lupus nephritis subjects. In some embodiments, the reference C3 level is a value that is equal to or less than the mean or median of C3 levels in samples derived from lupus nephritis subjects of a similar age. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects of a similar age. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects. In some embodiments, the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects of a similar age. In some embodiments, the normal or healthy subjects do not have lupus nephritis. In some embodiments, the reduced C3 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%. In some embodiments, the reduced C3 level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured in a biological sample. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured in plasma or urine. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 12 months before the administration of a therapeutically effective amount of an inhibitor of the classical complement pathway. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 6 months before the administration of an inhibitor. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 3 months before the administration of an inhibitor. In some embodiments, the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level, or the C3a level, or the C3a/C3 ratio, or the C3 level is measured less than about 1 month before the administration of an inhibitor.

In some embodiments, the subject has an elevated urine protein/creatinine ratio (UPCR) level. In some embodiments, the elevated UPCR level is greater than a UPCR level in normal or healthy subjects, is greater than a UPCR level in normal or healthy subjects of a similar age, or is greater than a reference UPCR level. The reference UPCR level may be equal to or greater than about 0.5 g/g, 1.0 g/g, 1.5 g/g, 2.0 g/g, 2.5 g/g, 3.0 g/g, 3.5 g/g, 4.0 g/g, 4.5 g/g, or 5.0. The elevated UPCR level may be greater than the UPCR level in normal or healthy subjects or the reference UPCR level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000%, 4000%, or 5000%. The elevated UPCR level may be greater than the UPCR level in normal or healthy subjects or the reference UPCR level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%. In some embodiments, the UPCR level is measured in urine.

In some embodiments, the classical complement inhibitor is a C1 inhibitor, such as a small molecule, an antibody, an aptamer, an antisense nucleic acid or a gene editing agent. In some embodiments, the antibody is a monoclonal antibody, a polyclonal antibody, a recombinant antibody, a humanized antibody, a chimeric antibody, a multispecific antibody, or an antibody derivative thereof.

In some embodiments, the inhibitor of the classical complement pathway is a C1q inhibitor, such as a small molecule, an antibody, an aptamer, an antisense nucleic acid or a gene-editing agent. In some embodiments, the antibody is a monoclonal antibody, a polyclonal antibody, a recombinant antibody, a humanized antibody, a chimeric antibody, a multispecific antibody, or an antibody derivative thereof. The antibody may be an anti-C1q antibody. In some embodiments, the antibody derivative thereof is a single arm antibody.

In some embodiments, the antibody is administered at a dose of at least 50 mg/kg, or at a dose between 50 mg/kg to 200 mg/kg. The antibody may be administered at a dose of 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, or 200 mg/kg. In some embodiments, the antibody is administered to a total of at least 50 mg. The antibody may be administered to a total dose of 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the antibody is administered daily, once a week, once every other week, once a month, once every six weeks, or once every other month. In some embodiments, the antibody is administered for at least 3 months, 6 months, at least 7 months, at least 8 months, at least 9 months, at least 10 months, at least 11 months, or at least 12 months, preferably for at least 6 months. The antibody may be administered throughout lifetime of the patient or intermittently throughout the lifetime of the patient, e.g., in response to a flare. In some embodiments, the antibody is administered at a dose of 75 mg/kg on day 1 and on day 5 or day 6. In some embodiments, the antibody is further administered at a dose of 100 mg/kg every two weeks. In some embodiments, the antibody is administered intravenously.

In some embodiments, the antibody is an antibody fragment, such as a Fab fragment, a Fab′ fragment, a F(ab′)2 fragment, a Fv fragment, a diabody, or a single chain antibody molecule. In some embodiments, the antibody fragment is administered to a total dose of at least 250 mg or to a total dose between 250 mg to 1000 mg. In some embodiments, the antibody fragment is administered to a total dose of 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg. In some embodiments, the antibody fragment is administered at a total dose of about 750 mg. In some embodiments, the antibody fragment is administered daily, once every other day, once every three days, once every four days, once every five days, or once every six days, once a week, once every two weeks, or once a month. In some embodiments, the antibody fragment is administered three times per week. In some embodiments, the antibody fragment is administered for at least 3 months, 6 months, at least 7 months, at least 8 months, at least 9 months, at least 10 months, at least 11 months, or at least 12 months. The antibody fragment may be administered throughout lifetime of the patient or intermittently throughout the lifetime of the patient, e.g., in response to a flare. In some embodiments, the antibody fragment is administered subcutaneously.

In some embodiments, the anti-C1q antibody inhibits the interaction between C1q and an autoantibody or between C1q and C1r, or between C1q and C1s and/or promotes clearance of C1q from circulation or a tissue. In some embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7, and/or a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, preferably the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11, preferably the heavy chain variable domain comprising an amino acid sequence selected from SEQ ID NO: 8 and 31-34. In some embodiments, the antibody comprises a light chain domain comprising the amino acid sequence of SEQ ID NO: 40; a first heavy chain domain comprising the amino acid sequence of SEQ ID NO: 2; and a second heavy chain domain comprising the amino acid sequence of SEQ ID NO: 3; the second heavy chain domain is an N-terminally truncated heavy chain. In some embodiments, the antibody fragment comprises heavy chain Fab fragment of SEQ ID NO: 39 and light chain Fab fragment of SEQ ID NO: 40.

In some embodiments, the inhibitor of the classical complement pathway is a C1r inhibitor, such as a small molecule, an antibody, an aptamer, an antisense nucleic acid or a gene editing agent, preferably an anti-C1r antibody. In some embodiments, the anti-C1r antibody inhibits the interaction between C1r and C1q or between C1r and C1s, or wherein the anti-C1r antibody inhibits the catalytic activity of C1r or inhibits the processing of pro-C1r to an active protease.

In some embodiments, the inhibitor of the classical complement pathway is a C1s inhibitor, such as a small molecule, an antibody, an aptamer, an antisense nucleic acid or a gene editing agent, preferably an anti-C1s antibody. In some embodiments, the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s. In some embodiments, the antibody is sutimlimab.

In some embodiments, the inhibitor of the classical complement pathway is an anti-C1 complex antibody, optionally wherein the anti-C1 complex antibody inhibits C1r or C1s activation or blocks their ability to act on C2 or C4. The anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s.

In some embodiments, the inhibitor of the classical complement pathway is a C2 inhibitor, such as a small molecule, an antibody, an aptamer, an antisense nucleic acid or a gene editing agent. The C2 inhibitor may be ARGX-117 (Argenx).

In some embodiments, the inhibitor of the classical complement pathway is a C3 inhibitor, such as a small molecule, an antibody, an aptamer, an antisense nucleic acid or a gene editing agent. The C3 inhibitor may be APL-9 (Apellis) or AMY-101 (Amyndas).

In some embodiments, the inhibitor of the classical complement pathway is a C4 inhibitor, such as a small molecule, an antibody, an aptamer, an antisense nucleic acid or a gene editing agent.

### EXAMPLES

### Example 1: Improved Model of Predicting and Treating Lupus

We observed evidence of complement activation in lupus nephritis patients relative to healthy controls. Specifically, levels of C4a, C4d, C1sC1 inhibitor, PACA1, PACA3 and the C4a/C4, C4d/C4, C1sC1 inhibitor/C1s, C2b/C2 and C3a/C3 ratios were highly increased in lupus nephritis patients, while levels of C4, C1s, C2, and C3 were decreased in lupus nephritis patients. More specifically, we observed levels of C4d and the C4d/C4 ratio were highly increased in lupus nephritis patients with flare, while levels of C1q, C1s, C2 and C4 were decreased, consistent with activation of the classical complement pathway (increased activation and component consumption). The C4d/C4 ratio also correlated with levels of autoantibodies against C1q isotypes 1 and 3 that are known to activate the classical pathway. These data indicated that a subset of LN patients exhibiting high C4d/C4 ratio along with specific markers of classical pathway activation are likely to benefit from a classical complement inhibitor, or more specifically an anti-C1q therapy. Moreover, these data suggest that a subset of LN patients exhibiting high levels of C4a, C4d, C1sC1 inhibitor, PACA1, PACA3, C4a/C4, are gC1sC1 inhibitor/C1s, and C2b/C2 or reduced levels of C4, C1s, C2 are likely to benefit from therapy with a classical complement inhibitor, or more specifically an anti-C1q therapy.

Table 1 shows the biomarker panel for plasma using ELISA.

| | | | | | | Classical, | | |
|---|---|---|---|---|---|---|---|---|
| | | | | Classical & | | Lectin and | Terminal | Autoimmune |
| Classical | | | | Lectin | | Alternative | Complement | specific |
| In | Free | C1s | C1 | C2 | C4 | C3 | C5 | PACA 1, 3 |
| house | C1q | | Inhibitor | | | | | |
| | Total | | | | | | | |
| | C1q | | | | | | | |
| | | C1q- | C1sC1in | C2b | C4a | C3a, C3d | C5a, C5b-9 | |
| | | C3d | complex | | | | | |
| | | IC | | | | | | |
| Kits | | | | | SVAR | | | |
| | | | | | C4d | | | |

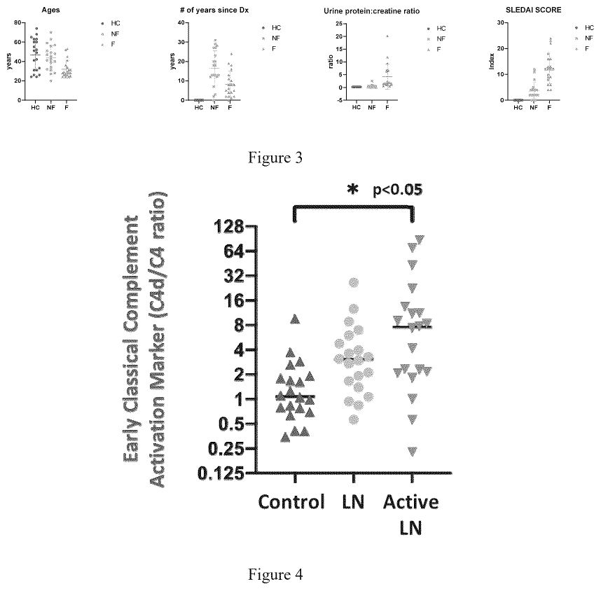

Patient demographic is shown in FIG. 3. SELENA-SLEDAI use an objective of overall disease activity. It assesses disease activity by scoring 24 weighted disease activity descriptors of SLE as “present” or “absent” in the preceding 10 days. See Bombardier C et al Arthritis Rheum 1992; Petri M. Ann Rheum Dis 2007; Petri et al N. Engl J Med 2005.

The following results were obtained. C4d/C4 ratio is elevated in lupus nephritis (LN) patients, suggesting role of the classical complement pathway in mediating disease. Classical complement pathway activation is enriched in LN. LN patients have an upregulation of C1 inhibitor, likely due to chronic complement activation. There is high correlation among complement factors in LN. PACA (Pathogenic Anti-C1q Antibody) levels correlate with classical complement activation in lupus nephritis patients. Improved C4d/C4 ratio post treatment further supports relevance of complement pathway in disease pathology. Clinical C4 measurements tracks UPCR (Urinary Protein/Creatine Ratio) trajectories. In this dataset C4d/C4 ratio is being driven low C4 (suggesting patients do not have high C4 copy number). In patients with high disease activity there is a temporal decline in C4d/C4 ratio. But their levels remain high relative to patients with low disease activity and healthy controls.

### Classical Complement Pathway Activation is Enriched in LN.

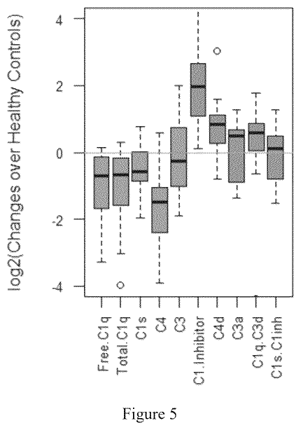

Higher circulating C4d/C4 ratio in patients with active LN suggests complement may drive disease progression and severity (FIG. 4). LN patients with high C4d/C4 ratio exhibit coordinated classical complement activation (FIG. 5). The data shows reduction of classical pathway substrates and increase in products in LN patients versus healthy controls.

In addition, higher circulating levels of C4a, C4d, C1sC1 inhibitor, PACA1, PACA3 and C2b/C2 ratio, C1sC1 inhibitor/C1s ratio and C3a/C3 ratio, as well as reduced levels of C4, C2, C1s, and C3 in patients with active LN suggests complement may drive disease progression and severity. (FIGS. 21A-O).

### High Correlation Among Complement Factors in LN.

High correlation among complement factors in LN patients suggests coordinated pathway activity (FIG. 6 and FIG. 22). There was no correlation observed in Health controls (FIG. 7). The data shows strong association between high C4d/C4 ratio and presence of PACA. The data also shows strong association between other complement factors in LN patients such as, for example, C4d/C4 and C4d have strong positive correlation, C2b and C2b/C2 have a strong anti-correlation (FIG. 22). The correlation is used to select for patients most likely to respond to therapy with a classical complement pathway, and more specifically with an anti-C1q therapy.

Paca Levels Correlate with Classical Complement Activation in Lupus Nephritis Patients

PACA1 levels remain elevated throughout treatment course. Active LN patients with high C4d/C4 ratio are likely to have higher PACA1 (FIGS. 8A-8B). PACA1 likely serves as the additive factor for amplifying disease severity. A subset of patients with high PACA do not have low C4.

Improved C4d/C4 Ratio Post Treatment further Supports Relevance of Complement Pathway in Disease Pathology

C4d/C4 is reduced post flare event by standard of care but effect is not durable (FIG. 9). FIG. 10 shows changes in urine protein/creatinine ratio (UPCR) and C4 in response to standard of care treatment. Rising level of C4 is approximately equivalent to reduction of C4d/C4 (C4d is novel, not routinely monitored). Rising level of C4 coincides with improved UPCR. Falling level of C4 post flare episode suggests residual inflammation that may lead to flare recurrence.

Anti-C1q treatment, such as for example, Anti-C1q Fab (e.g., FabA), has an opportunity to improve depth and durability of response in these patients by preventing activation of complement. Anti-C1q Fab, FabA, may improve proportion of complete responders by targeting classical complement directly.

The data also shows that clinical C4 measurements tracks UPCR trajectories (FIG. 11). Rise in C4 and decreased C4d/C4 correlates with improved disease activity (FIG. 12).

### Summary

In a cohort of 40 LN patients, C4a, C4d, C1sC1 inhibitor, C3a, PACA1, PACA3 and C4a/C4, C4d/C4, C2b/C2, C3a/C3, and C1sC1 inhibitor/C1s ratios were found higher than healthy controls, particularly those with active disease, indicating involvement of classical complement activation in disease pathology. In addition, C4, C1s, C2 and C3 were found lower than healthy controls, particularly those with active disease, also indicating involvement of classical complement activation in disease pathology. There is correlative evidence that PACA1 amplifies disease severity. Standard of care does not normalize C4d/C4 in non-responders, suggesting existing treatment not addressing underlying disease cause in those patients. Patients with high C4d/C4 benefits from a therapy that target C1q, inhibiting the activation of the disease-mediated pathway. Selecting patients with C4d/C4 opens opportunity to: target patients with complement activation as key disease driver, thus most likely to achieve improved outcome; and identify patients in earlier disease state, likely to achieve superior outcome. Selecting patients with at least elevated C4a, C4d, C1sC1 inhibitor, PACA1, PACA3 and C4a/C4 ratio, C2b/C2 ratio and C1sC1 inhibitor/C1s ratio, as well as reduced C4, C2 and C1s levels also opens the opportunity to: target patients with complement activation as key disease driver, thus most likely to achieve improved outcome; and identify patients in earlier disease state, likely to achieve superior outcome.

### Example 2: a Single-Arm, Phase 1b, Open-Label Study to Assess the Safety, Tolerability, and Pharmacodynamics of Repeat-Doses of Subcutaneous FabA with Standard of Care Therapy in Adult Participants with Lupus Nephritis

The study consists of an up to 8-week (56-day) Screening Period, an approximate 3-week (22-day) Intervention Period, and an approximate 11-week (80-day) off-treatment Follow-up Period. The maximum duration of participation is approximately 158 days (˜23 weeks). Participants receive FabA 750 mg via SC infusion pump 3 times a week, with not more than 3 days between doses. The total dose administered in this LN study is within the safety margins established in the nonclinical toxicology studies. Notably, no safety findings were observed in nonclinical 4-week GLP toxicology studies in cynomolgus monkeys at SC doses up to the human equivalent of 1500 mg daily (no observed adverse effect level [NOAEL] of 20 mg/kg daily, the highest dose tested), during which complete C1q suppression was observed in serum for the duration of the dosing interval.

Screening visits (Week-8, Week-6, and Week-2): All participants undergo study screening procedures within 56 days prior to dosing with FabA. Renal biopsy is required for those participants who have not had one within 24 months of screening.

The study investigates the safety and PD effects of FabA administered via SC infusion pump in participants with ISN/RPS Class III/IV (±V) LN. Participants who demonstrate proteinuria in the range of 0.5-3.0 g/g/day using UPCR and evidence of classical complement activation (high C4d/C4 ratio) receive FabA in addition to stable standard of care therapy. This population is considered to have incomplete renal response despite previous induction therapy with treatments such as cyclophosphamide, MMF, rituximab, and glucocorticoids.

High C4d/C4 ratio is used to identify LN participants with classical complement pathway activation and ongoing kidney inflammation. Approximately half of LN patients with high C4d/C4 ratio have proteinuria in the range of 0.5-3.0 g/g/day (the rest have proteinuria >3.0 g/g/day), suggesting the presence of residual kidney disease activity.

Samples (saliva or an existing blood sample) are collected for genetic research related to this study.

All participants receive FabA 750 mg SC 3 times per week for approximately 3 weeks (10 doses) administered via a SC infusion pump, with not more than 3 days between doses.

### Example 3: PACA Plasma Titers Correlate with the Degree of Kidney Damage in Lupus Nephritis Patients

PACA plasma titers correlate with the degree of kidney damage in lupus nephritis patients, as assessed by the urinary protein/creatinine ratio (UPCR) as a measure of proteinuria. PACAs of the IgG1 and IgG3 isotypes provided the strongest degree of correlation with the UPCR, whereas there was no significant correlation with levels of IgG2 and IgG4 antibody against C1q (FIG. 13). These results are important because IgG1 and IgG3 are the antibody isotypes that most strongly recruit additional C1q (complement fixing isotypes), whereas IgG2 and IgG4 would recruit little or no C1q to the immune complex, and hence are not associated with an increased level of disease. While IgM antibodies strongly activate C1q, they are characteristic of acute disease and would not be expected to circulate at appreciable levels in a chronic disorder such as lupus nephritis. PACA1 and PACA3 levels also correlate with C4d and the C4d/C4 ratio (FIG. 14).

Moreover, early study with animal model of lupus nephritis reports causal link between anti-C1q antibodies and kidney damage. Administration of PACA greatly exacerbates disease with large increase in complement deposition in glomerular subendothelial space (FIGS. 17 and 18). PACAs are also characterized by ability to selectively bind substrate-bound C1q and activate classical complement pathway. PACA binding to substrate-bound C1q is not affected by soluble C1q (FIG. 19). Accordingly, PACA correlate with disease severity and are complement-activating IgG1 and IgG3 isotypes vs IgG4, which does not fix C1q. Anti-C1q IgG1 and IgG3, but not IgG4 levels correlate with serum C4d/C4 ratio (FIG. 20). This is consistent with complement activating capability of the antibody isotypes.

Complement factor C4 and its activation product C4d are important biomarkers in LN. Reduced levels of C4 and increased amounts of C4d in the circulation signal activation of the classical complement pathway and are associated with active LN. C4d levels in plasma are strongly associated with C4d deposition in kidney tissue from active LN patients. C4d/C4 ratio is elevated in active LN, compared with SLE patients without LN, patients with autoimmune kidney disease IgA nephropathy, and healthy controls. Further, C4d/C4 ratio accurately predicts the presence of active LN to a higher degree than other biomarkers (e.g., C3, C4, and C4d). C4d/C4 may therefore identify active LN patients who are most likely to respond to therapies targeting the classical complement cascade.

Together, this data provides strong scientific and clinical rationale for evaluating anti-C1q therapeutics in patients with Class III/IV LN who have evidence of classical complement pathway activation.

### Example 4: a Single-Arm. Phase 1b, Open-Label Study to Assess the Safety, Tolerability, and Pharmacodynamics of Repeat-Doses of Subcutaneous FabA with Standard of Care Therapy in Adult Participants with Lupus Nephritis

The study consists of an up to 8-week (56-day) Screening Period, an approximate 3-week (22-day) Intervention Period, and an approximate 11-week (80-day) off-treatment Follow-up Period. The maximum duration of participation is approximately 158 days (˜23 weeks). Participants receive FabA 750 mg via SC infusion pump 3 times a week, with not more than 3 days between doses. The total dose administered in this LN study is within the safety margins established in the nonclinical toxicology studies. Notably, no safety findings were observed in nonclinical 4-week GLP toxicology studies in cynomolgus monkeys at SC doses up to the human equivalent of 1500 mg daily (no observed adverse effect level [NOAEL] of 20 mg/kg daily, the highest dose tested), during which complete C1q suppression was observed in serum for the duration of the dosing interval.

Screening visits (Week-8, Week-6, and Week-2): All participants undergo study screening procedures within 56 days prior to dosing with FabA. Renal biopsy is required for those participants who have not had one within 24 months of screening.

The study investigates the safety and PD effects of FabA administered via SC infusion pump in participants with ISN/RPS Class III/IV (±V) LN. Participants who demonstrate proteinuria in the range of 0.5-3.0 g/g/day using UPCR and evidence of classical complement activation (high C4d/C4 ratio) receive FabA in addition to stable standard of care therapy. This population is considered to have incomplete renal response despite previous induction therapy with treatments such as cyclophosphamide, MMF, rituximab, and glucocorticoids.

High C4d/C4 ratio is used to identify LN participants with classical complement pathway activation and ongoing kidney inflammation. High C4d/C4 ratio in this study is defined as an LN patient having a C4d/C4 ratio that is equal to or greater than the medium in samples derived from LN subjects. Approximately half of LN patients with high C4d/C4 ratio have proteinuria in the range of 0.5-3.0 g/g/day (the rest have proteinuria >3.0 g/g/day), suggesting the presence of residual kidney disease activity.

Samples (saliva or an existing blood sample) are collected for genetic research related to this study.

All participants receive FabA 750 mg SC 3 times per week for approximately 3 weeks (10 doses) administered via a SC infusion pump, with not more than 3 days between doses.

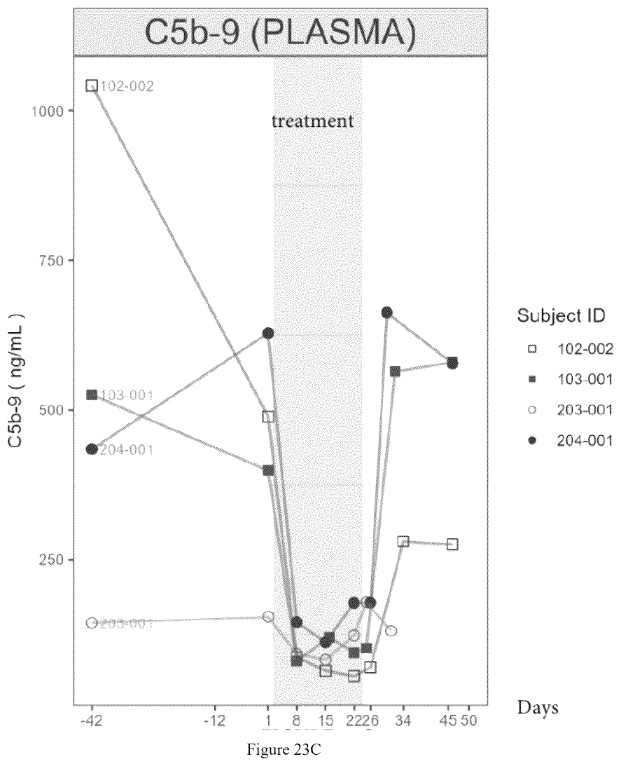

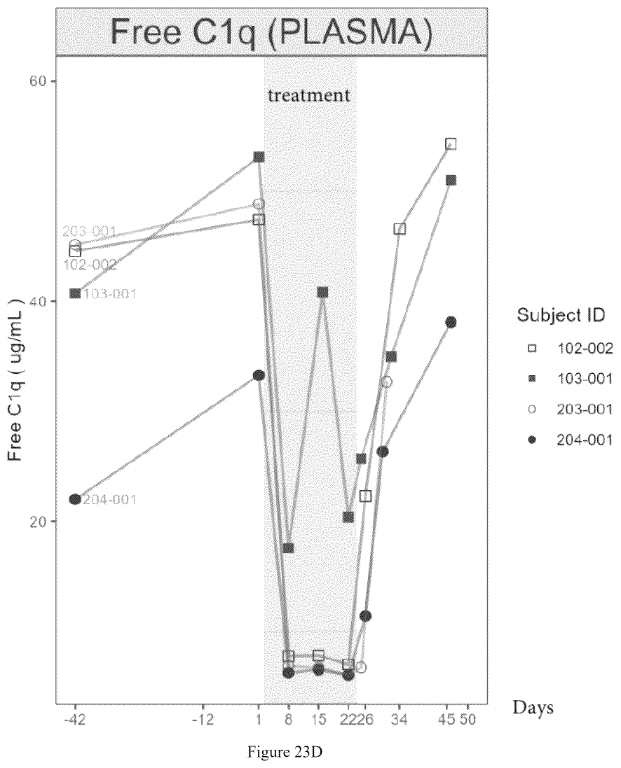

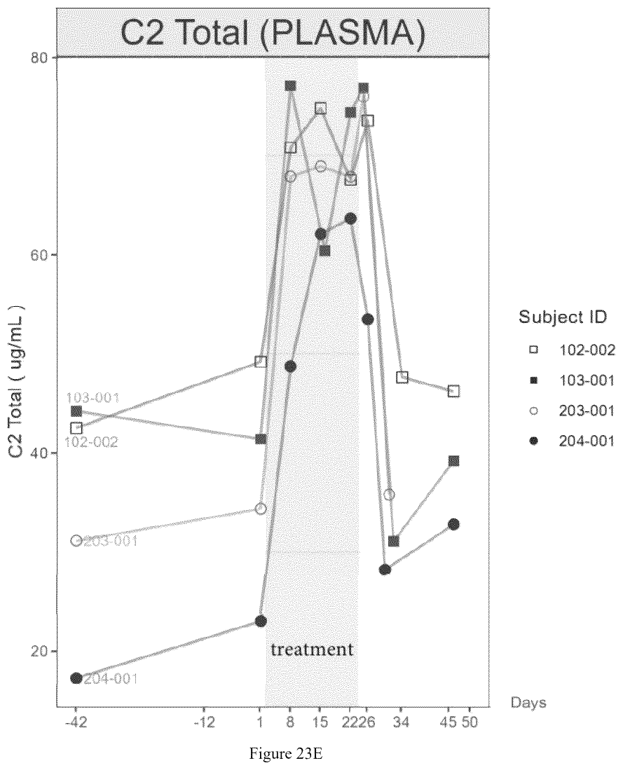

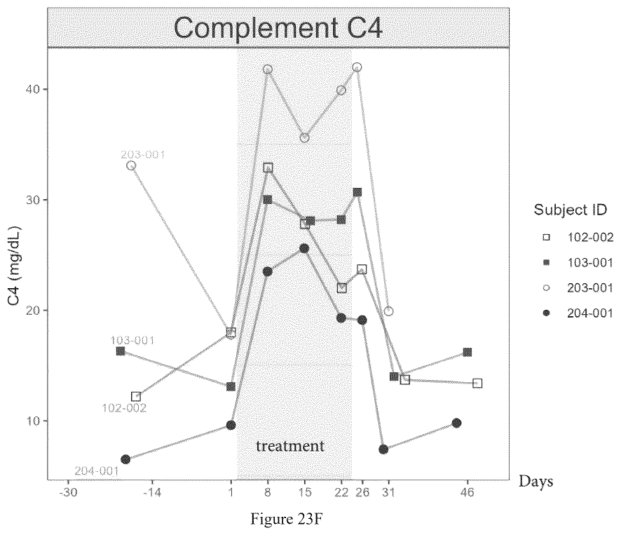

Four patients completed treatment and screening is ongoing. Interim data showed that the C4d/C4 ratio decreased with treatment in all four patients, and returned to baseline after cessation (FIG. 23A). In addition, inhibition of C1q resulted in normalization of downstream markers, including C3 and C59b, indicating that the classical pathway is an important driver of complement activation in these LN patients (FIGS. 23B-F). No consistent trends in urine protein creatinine ratio were observed in this short treatment study.

In this interim analysis, FabA administered subcutaneously was well tolerated and demonstrated C1q target engagement and complement inhibition in four patients. In addition, normalization of all downstream components, including C3 and C59-b suggests that the classical pathway is a key driver of complement activation in these LN patients. These interim results support the use of anti-C1q therapy in a subset of LN patients.

### Example 5: General Methods

Complement Protein C4d is measured using the SVAR C4d ELISA Kit (SVAR, COMPL C4d RUO). Levels of PACA1, PACA3, and complement proteins C1s, C1sC1inh, C2, C2b, C4, C4a, C3, C3a, and C3d are measured in plasma samples using sandwich ELISA.

Black 96 well plates (Costar #3925) are coated with 75 μL of 2-3 μg/mL capture antibody in bicarbonate buffer (pH 9.4) overnight at 4° C. Next day, the plates are washed with dPBS (pH 7.4) and blocked with dPBS buffer containing 3% bovine serum albumin (BSA). Standards for complement proteins C1s, C1sC1inh, C2, C2b, C4, C4a, C3, C3a, and C3d are purchased from Complement Tech and is prepared in the ranges of 100-0.045, 1000-0.45, 200-0.09, 30-0.013, 100-0.04, 500-0.22, 100-0.045, 0.5-0.004, and 50-0.002 ng/mL in dPBS containing 0.3% BSA, 0.01 M EDTA, and 0.1% tween (Assay Buffer, respectively). Standards for PACA1 (ATUM, 63044) and PACA3 (ATUM, 63050), are prepared in the range of 300-0.02 ng/mL in dPBS containing 0.3% BSA, 0.01 M EDTA, 0.1% tween, and IM NaCl (High Salt Assay Buffer). Plasma samples are diluted in the ranges of 30000 (C1s), 90 (C1sC1inh), 8000 (C2), 2000 (C2b), 400,000 (C4), 200 (C4a), 200000 (C3), 50000 (C3a), 200000 (C3d) in assay buffer and 20 (PACA1, PACA3) in the above-mentioned high salt assay buffer.

Following an hour of incubation the blocking buffer is removed from the plates. Standards and samples are added at 75 μL per well and incubated shaking at 300 rpm overnight at 4° C. The plates are then washed thrice with dPBS containing 0.05% Tween. Then, 75 μL of appropriate alkaline-phosphatase conjugated detection antibody is added to all wells. Plates are incubated for 1 hr shaking at room temperature, washed thrice with dPBS containing 0.05% Tween and then developed using 75 μL of alkaline phosphatase substrate (Life Technologies, T2214). After incubation for 20 minutes, plates are read using a luminometer. Standards are fit using a 4PL logistic fit and unknowns converted to concentration, corrected for dilution, and then plotted using the appropriate plotting software, which in some of these examples is GraphPad Prism.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed is:

1. A method of treating lupus nephritis in a subject in need thereof, the method comprising: determining that the subject has at least one of the following characteristics:
a. an elevated C4x level;
b. an elevated C4x/C4 ratio;
c. a reduced C4 level;
d. an elevated C1sC1 inhibitor level;
e. an elevated C1sC1 inhibitor/C1s ratio;
f a reduced C1s level;
g an elevated C2b level;
h. an elevated C2b/C2 ratio;
i. a reduced C2 level; or
j. an elevated Pathogenic Anti-C1q Antibody 1 (PACA1) and/or Pathogenic Anti-C1q Antibody 3 (PACA3) level;
wherein C4x is selected from the group consisting of C4a, and C4d, and
administering to the subject an inhibitor of the classical complement pathway.

2. The method of claim 1, wherein the subject has at least two of the characteristics.

3. The method of claim 2, wherein the characteristics are selected from elevated C4x levels, elevated C4x/C4 ratio and reduced C4 levels.

4. The method of claim 2, wherein the characteristics are selected from elevated C1sC1 inhibitor levels, elevated C1sC1 inhibitor/C1s ratio and reduced C1s levels.

5. The method of claim 3 or 4, wherein the subject further has an elevated Pathogenic Anti-C1q Antibody 1 (PACA1) and/or Pathogenic Anti-C1q Antibody 3 (PACA3) level.

6. The method of any one of claims 1-5, wherein the subject has at least one of the following additional characteristics:
a. an elevated C3a level;
b. an elevated C3a/C3 ratio; or
c. a reduced C3 level.

7. The method of claim 1, wherein the subject has an elevated C4x level.

8. The method of claim 7, wherein the subject has at least one of the following additional characteristics:
a. an elevated C4x/C4 ratio;
b. a reduced C4 level;
c. an elevated C1sC1 inhibitor level;
d. an elevated C1sC1 inhibitor/C1s ratio;
e. a reduced C1s level;
f an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j an elevated C3a level;
k. an elevated C3a/C3 ratio; or
l. a reduced C3 level.

9. The method of any one of claims 1-8, wherein the elevated C4x level is greater than a C4x level in normal or healthy subjects.

10. The method of any one of claims 1-8, wherein the elevated C4x level is greater than a C4x level in normal or healthy subjects of a similar age.

11. The method of any one of claims 1-10, wherein the elevated C4x level is greater than a reference C4x level.

12. The method of claim 11, wherein the reference C4x level is a value that is equal to or greater than the mean or median of C4x levels in samples derived from lupus nephritis subjects.

13. The method of claim 11, wherein the reference C4x level is a value that is equal to or greater than the mean or median of C4x levels in samples derived from lupus nephritis subjects of a similar age.

14. The method of claim 11, wherein the reference C4x level is a value that is equal to or greater than the 55th percentile, 60th percentile, 65th percentile, 70th percentile, 75th percentile, 80th percentile, 85th percentile, 90th percentile, 95th percentile, or 100th percentile of C4x levels in samples derived from lupus nephritis subjects.

15. The method of claim 11, wherein the reference C4x level is a value that is equal to or greater than the 55th percentile, 60th percentile, 65th percentile, 70th percentile, 75th percentile, 80th percentile, 85th percentile, 90th percentile, 95th percentile, or 100th percentile of C4x levels in samples derived from lupus nephritis subjects of a similar age.

16. The method of claim 11, wherein the reference C4x level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C4x levels in samples derived from normal or healthy subjects.

17. The method of claim 11, wherein the reference C4x level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C4x levels in samples derived from normal or healthy subjects of a similar age.

18. The method of any one of claims 12-17, wherein the normal or healthy subjects do not have lupus nephritis.

19. The method of any one of claims 7-18, wherein the elevated C4x level is greater than the reference C4x level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

20. The method of any one of claims 7-18, wherein the elevated C4x level is greater than the reference C4x level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

21. The method of claim 1, wherein the subject has an elevated C4x/C4 ratio.

22. The method of claim 21, wherein the subject has at least one of the following additional characteristics:
a. an elevated C4x level;
b. a reduced C4 level;
c. an elevated C1sC1 inhibitor level;
d. an elevated C1sC1 inhibitor/C1s ratio;
e. a reduced C1s level;
f an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

23. The method of any one of claims 1-22, wherein the elevated C4x/C4 ratio is greater than a C4x/C4 ratio in normal or healthy subjects.

24. The method of any one of claims 1-22, wherein the elevated C4x/C4 ratio is greater than a C4x/C4 ratio in normal or healthy subjects of a similar age.

25. The method of any one of claims 1-24, wherein the elevated C4x/C4 ratio is greater than a reference C4x/C4 ratio.

26. The method of claim 25, wherein the reference C4x/C4 ratio is a value that is equal to or greater than the mean or median of C4x/C4 ratio in samples derived from lupus nephritis subjects.

27. The method of claim 25, wherein the reference C4x/C4 ratio is a value that is equal to or greater than the mean or median of C4x/C4 ratio in samples derived from lupus nephritis subjects of a similar age.

28. The method of claim 25, wherein the reference C4x/C4 ratio is a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects.

29. The method of claim 25, wherein the reference C4x/C4 ratio is a value that is equal to or greater than the 55th percentile, 60th percentile, 65th percentile, 70th percentile, 75th percentile, 80th percentile, 85th percentile, 90th percentile, 95th percentile, or 100th percentile of C4x/C4 ratio in samples derived from lupus nephritis subjects of a similar age.

30. The method of claim 25, wherein the reference C4x/C4 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C4x/C4 ratio in samples derived from normal or healthy subjects.

31. The method of claim 25, wherein the reference C4x/C4 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C4x/C4 ratio in samples derived from normal or healthy subjects of a similar age.

32. The method of any one of claim 26-31, wherein the normal or healthy subjects do not have lupus nephritis.

33. The method of any one of claims 25-32, wherein the elevated C4x/C4 ratio is greater than the reference C4x/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

34. The method of any one of claims 25-32, wherein the elevated C4x/C4 ratio is greater than the reference C4x/C4 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

35. The method of claim 1, wherein the subject has a reduced C4 level.

36. The method of claim 35, wherein the subject has at least one of the following additional characteristics:
a. an elevated C4x level;
b. an elevated C4x/C4 ratio;
c. an elevated C1sC1 inhibitor level;
d. an elevated C1sC1 inhibitor/C1s ratio;
e. a reduced C1s level;
f. an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

37. The method of any one of claims 1-36, wherein the reduced C4 level is less than a C4 level in normal or healthy subjects.

38. The method of any one of claims 1-36, wherein the reduced C4 level is less than a C4 level in normal or healthy subjects of a similar age.

39. The method of any one of claims 36-38, wherein the reduced C4 level is less than a reference C4 level.

40. The method of claim 39, wherein the reference C4 level is a value that is equal to or less than the mean or median of C4 levels in samples derived from lupus nephritis subjects.

41. The method of claim 39, wherein the reference C4 level is a value that is equal to or less than the mean or median of C4 levels in samples derived from lupus nephritis subjects of a similar age.

42. The method of claim 39, wherein the reference C4 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects.

43. The method of claim 39, wherein the reference C4 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 level in samples derived from lupus nephritis subjects of a similar age.

44. The method of claim 39, wherein the reference C4 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects.

45. The method of claim 39, wherein the reference C4 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C4 levels in samples derived from normal or healthy subjects of a similar age.

46. The method of any one of claims 40-45, wherein the normal or healthy subjects do not have lupus nephritis.

47. The method of any one of claims 35-46, wherein the reduced C4 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

48. The method of any one of claims 35-46, wherein the reduced C4 level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

49. The method of claim 1, wherein the subject has an elevated C1sC1 inhibitor level.

50. The method of claim 49, wherein the subject has at least one of the following additional characteristics:
a. an elevated C4x level;
b. an elevated C4x/C4 ratio;
c. a reduced C4 level;
d. an elevated C1sC1 inhibitor/C1s ratio;
e. a reduced C1s level;
f an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

51. The method of any one of claims 1-50, wherein the elevated C1sC1 inhibitor level is greater than a C1sC1 inhibitor level in normal or healthy subjects.

52. The method of any one of claims 1-50, wherein the elevated C1sC1 inhibitor level is greater than a C1sC1 inhibitor level in normal or healthy subjects of a similar age.

53. The method of any one of claims 1-52, wherein the elevated C1sC1 inhibitor level is greater than a reference C1sC1 inhibitor level.

54. The method of claim 53, wherein the reference C1sC1 inhibitor level is a value that is equal to or greater than the mean or median of C1sC1 inhibitor levels in samples derived from lupus nephritis subjects.

55. The method of claim 53, wherein the reference C1sC1 inhibitor level is a value that is equal to or greater than the mean or median of C1sC1 inhibitor levels in samples derived from lupus nephritis subjects of a similar age.

56. The method of claim 53, wherein the reference C1sC1 inhibitor level is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects.

57. The method of claim 53, wherein the reference C1sC1 inhibitor level is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor level in samples derived from lupus nephritis subjects of a similar age.

58. The method of claim 53, wherein the reference C1sC1 inhibitor level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor levels in samples derived from normal or healthy subjects.

59. The method of claim 53, wherein the reference C1sC1 inhibitor level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor levels in samples derived from normal or healthy subjects of a similar age.

60. The method of any one of claims 51-59, wherein the normal or healthy subjects do not have lupus nephritis.

61. The method of any one of claims 53-60, wherein the elevated C1sC1 inhibitor level is greater than the reference C1sC1 inhibitor level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

62. The method of any one of claims 53-60, wherein the elevated C1sC1 inhibitor level is greater than the reference C1sC1 inhibitor level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

63. The method of claim 1, wherein the subject has an elevated C1sC1 inhibitor/C1s ratio.

64. The method of claim 63, wherein the subject has at least one of the following additional characteristics:
a. an elevated C4x level;
b. a reduced C4 level;
c. an elevated C4x/C4 ratio;
d. an elevated C1sC1 inhibitor level;
e. a reduced C1s level;
f. an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

65. The method of any one of claims 1-64, wherein the elevated C1sC1 inhibitor/C1s ratio is greater than a C1sC1 inhibitor/C1s ratio in normal or healthy subjects.

66. The method of any one of claims 1-65, wherein the elevated C1sC1 inhibitor/C1s ratio is greater than a C1sC1 inhibitor/C1s ratio in normal or healthy subjects of a similar age.

67. The method of any one of claims 1-64, wherein the elevated C1sC1 inhibitor/C1s ratio is greater than a reference C1sC1 inhibitor/C1s ratio.

68. The method of claim 67, wherein the reference C1sC1 inhibitor/C1s ratio is a value that is equal to or greater than the mean or median of C1sC1 inhibitor/C1s ratio in samples derived from lupus nephritis subjects.

69. The method of claim 67, wherein the reference C1sC1 inhibitor/C1s ratio is a value that is equal to or greater than the mean or median of C1sC1 inhibitor/C1s ratio in samples derived from lupus nephritis subjects of a similar age.

70. The method of claim 67, wherein the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from lupus nephritis subjects.

71. The method of claim 67, wherein the reference C1sC1 inhibitor/C1s ratio is a value that is equal to or greater than the 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from lupus nephritis subjects of a similar age.

72. The method of claim 67, wherein the reference C1sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from normal or healthy subjects.

73. The method of claim 67, wherein the reference C1sC1 inhibitor/C1s ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C1sC1 inhibitor/C1s ratio in samples derived from normal or healthy subjects of a similar age.

74. The method of any one of claim 65-73, wherein the normal or healthy subjects do not have lupus nephritis.

75. The method of any one of claims 67-74, wherein the elevated C1sC1 inhibitor/C1s ratio is greater than the reference C1sC1 inhibitor/C1s ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

76. The method of any one of claims 67-74, wherein the elevated C1sC1 inhibitor/C1s ratio is greater than the reference C1sC1 inhibitor/C1s ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

77. The method of claim 1, wherein the subject has a reduced C1s level.

78. The method of claim 77, wherein the subject has at least one of the following additional characteristics:
a. an elevated C4x level;
b. an elevated C4x/C4 ratio;
c. a reduced C4 level;
d. an elevated C1sC1 inhibitor/C1s ratio;
e. an elevated C1sC1 inhibitor level;
f. an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

79. The method of any one of claims 1-78, wherein the reduced C1s level is less than a C1s level in normal or healthy subjects.

80. The method of any one of claims 1-78, wherein the reduced C1s level is less than a C1s level in normal or healthy subjects of a similar age.

81. The method of any one of claims 77-80, wherein the reduced C1s level is less than a reference C1s level.

82. The method of claim 81, wherein the reference C1s level is a value that is equal to or less than the mean or median of C1s levels in samples derived from lupus nephritis subjects.

83. The method of claim 81, wherein the reference C1s level is a value that is equal to or less than the mean or median of C1s levels in samples derived from lupus nephritis subjects of a similar age.

84. The method of claim 81, wherein the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s level in samples derived from lupus nephritis subjects.

85. The method of claim 81, wherein the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s level in samples derived from lupus nephritis subjects of a similar age.

86. The method of claim 81, wherein the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects.

87. The method of claim 81, wherein the reference C1s level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C1s levels in samples derived from normal or healthy subjects of a similar age.

88. The method of any one of claims 79-87, wherein the normal or healthy subjects do not have lupus nephritis.

89. The method of any one of claims 81-88, wherein the reduced C1s level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

90. The method of any one of claims 81-88, wherein the reduced C1s level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

91. The method of claim 1, wherein the subject has an elevated C2b level.

92. The method of claim 91, wherein the subject has at least one of the following additional characteristics:
a. an elevated C4x level;
b. an elevated C4x/C4 ratio;
c. a reduced C4 level;
d. an elevated C1sC1 inhibitor/C1s ratio;
e. an elevated C1sC1 inhibitor level;
f. a reduced C1s level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

93. The method of any one of claims 1-92, wherein the elevated C2b level is greater than a C2b level in normal or healthy subjects.

94. The method of any one of claims 1-93, wherein the elevated C2b level is greater than a C2b level in normal or healthy subjects of a similar age.

95. The method of any one of claims 1-94, wherein the elevated C2b level is greater than a reference C2b level.

96. The method of claim 95, wherein the reference C2b level is a value that is equal to or greater than the mean or median of C2b levels in samples derived from lupus nephritis subjects.

97. The method of claim 95, wherein the reference C2b level is a value that is equal to or greater than the mean or median of C2b levels in samples derived from lupus nephritis subjects of a similar age.

98. The method of claim 95, wherein the reference C2b level is a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects.

99. The method of claim 95, wherein the reference C2b level is a value that is equal to or greater than the 55th percentile, 60th percentile, 65th percentile, 70th percentile, 75th percentile, 80th percentile, 85th percentile, 90th percentile, 95th percentile, or 100th percentile of C2b level in samples derived from lupus nephritis subjects of a similar age.

100. The method of claim 95, wherein the reference C2b level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b levels in samples derived from normal or healthy subjects.

101. The method of claim 95, wherein the reference C2b level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b levels in samples derived from normal or healthy subjects of a similar age.

102. The method of any one of claims 92-11, wherein the normal or healthy subjects do not have lupus nephritis.

103. The method of any one of claims 95-102, wherein the elevated C2b level is greater than the reference C2b level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

104. The method of any one of claims 95-102, wherein the elevated C2b level is greater than the reference C2b level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

105. The method of claim 1, wherein the subject has an elevated C2b/C2 ratio.

106. The method of claim 105, wherein the subject has at least one of the following additional characteristics:
a. an elevated C4x level;
b. an elevated C4x/C4 ratio;
c. a reduced C4 level;
d. an elevated C1sC1 inhibitor/C1s ratio;
e. an elevated C1sC1 inhibitor level;
f. a reduced C1s level;
g. a reduced C2 level;
h. an elevated C2b level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

107. The method of any one of claims 1-106, wherein the elevated C2b/C2 ratio is greater than a C2b/C2 ratio in normal or healthy subjects.

108. The method of any one of claims 1-107, wherein the elevated C2b/C2 ratio is greater than a C2b/C2 ratio in normal or healthy subjects of a similar age.

109. The method of any one of claims 1-108, wherein the elevated C2b/C2 ratio is greater than a reference C2b/C2 ratio.

110. The method of claim 109, wherein the reference C2b/C2 ratio is a value that is equal to or greater than the mean or median of C2b/C2 ratio in samples derived from lupus nephritis subjects.

111. The method of claim 109, wherein the reference C2b/C2 ratio is a value that is equal to or greater than the mean or median of C2b/C2 ratio in samples derived from lupus nephritis subjects of a similar age.

112. The method of claim 109, wherein the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects.

113. The method of claim 109, wherein the reference C2b/C2 ratio is a value that is equal to or greater than the 55th percentile, 60th percentile, 65th percentile, 70th percentile, 75th percentile, 80th percentile, 85th percentile, 90th percentile, 95th percentile, or 100th percentile of C2b/C2 ratio in samples derived from lupus nephritis subjects of a similar age.

114. The method of claim 109, wherein the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects.

115. The method of claim 109, wherein the reference C2b/C2 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C2b/C2 ratio in samples derived from normal or healthy subjects of a similar age.

116. The method of any one of claim 107-115, wherein the normal or healthy subjects do not have lupus nephritis.

117. The method of any one of claims 109-116, wherein the elevated C2b/C2 ratio is greater than the reference C2b/C2 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

118. The method of any one of claims 109-116, wherein the elevated C2b/C2 ratio is greater than the reference C2b/C2 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

119. The method of claim 1, wherein the subject has a reduced C2 level.

120. The method of claim 119, wherein the subject has at least one of the following additional characteristics:
a. an elevated C4x level;
b. an elevated C4x/C4 ratio;
c. a reduced C4 level;
d. an elevated C1sC1 inhibitor/C1s ratio;
an elevated C1sC1 inhibitor level;
f. a reduced C1s level;
g. an elevated C2b level;
h. an elevated C2b/C2 ratio;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio; or
l. a reduced C3 level.

121. The method of any one of claims 1-120, wherein the reduced C2 level is less than a C2 level in normal or healthy subjects.

122. The method of any one of claims 1-120, wherein the reduced C2 level is less than a C2 level in normal or healthy subjects of a similar age.

123. The method of any one of claims 1-122, wherein the reduced C2 level is less than a reference C2 level.

124. The method of claim 123, wherein the reference C2 level is a value that is equal to or less than the mean or median of C2 levels in samples derived from lupus nephritis subjects.

125. The method of claim 123, wherein the reference C2 level is a value that is equal to or less than the mean or median of C2 levels in samples derived from lupus nephritis subjects of a similar age.

126. The method of claim 123, wherein the reference C2 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects.

127. The method of claim 123, wherein the reference C2 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 level in samples derived from lupus nephritis subjects of a similar age.

128. The method of claim 123, wherein the reference C2 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects.

129. The method of claim 123, wherein the reference C2 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C2 levels in samples derived from normal or healthy subjects of a similar age.

130. The method of any one of claims 122-129, wherein the normal or healthy subjects do not have lupus nephritis.

131. The method of any one of claims 123-130, wherein the reduced C2 level is less than the reference C2 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

132. The method of any one of claims 123-130, wherein the reduced C2 level is less than the reference C2 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

133. The method of claim 1, wherein the subject has an elevated Pathogenic Anti-C1q Antibody 1 (PACA1) and/or Pathogenic Anti-C1q Antibody 3 (PACA3) level.

134. The method of claim 133, wherein the subject has at least one of the following additional characteristics:
a. an elevated C4x level;
b an elevated C4x/C4 ratio;
c. a reduced C4 level;
d. an elevated C1sC1 inhibitor/C1s ratio;
e. an elevated C1sC1 inhibitor level;
f. a reduced C1s level;
g an elevated C2b level;
h. an elevated C2b/C2 ratio;
i. a reduced C2 level;
j. an elevated C3a level;
k. an elevated C3a/C3 ratio; or
l. a reduced C3 level.

135. The method of any one of claims 1-134, wherein the elevated PACA1 and/or PACA3 level is greater than a PACA1 and/or PACA3 level in normal or healthy subjects.

136. The method of any one of claims 1-134, wherein the elevated PACA1 and/or PACA3 level is greater than a PACA1 and/or PACA3 level in normal or healthy subjects of a similar age.

137. The method of any one of claims 1-136, wherein the elevated PACA1 and/or PACA3 level is greater than a reference PACA1 and/or PACA3 level.

138. The method of claim 137, wherein the reference PACA1 and/or PACA3 level is a value that is equal to or greater than the mean or median of PACA1 and/or PACA3 levels in samples derived from lupus nephritis subjects.

139. The method of claim 137, wherein the reference PACA1 and/or PACA3 level is a value that is equal to or greater than the mean or median of PACA1 and/or PACA3 levels in samples derived from lupus nephritis subjects of a similar age.

140. The method of claim 137, wherein the reference PACA1 and/or PACA3 level is a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects.

141. The method of claim 137, wherein the reference PACA1 and/or PACA3 level is a value that is equal to or greater than the 55th percentile, 60th percentile, 65th percentile, 70th percentile, 75th percentile, 80th percentile, 85th percentile, 90th percentile, 95th percentile, or 100th percentile of PACA1 and/or PACA3 level in samples derived from lupus nephritis subjects of a similar age.

142. The method of claim 137, wherein the reference PACA1 and/or PACA3 level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of PACA1 and/or PACA3 levels in samples derived from normal or healthy subjects.

143. The method of claim 137, wherein the reference PACA1 and/or PACA3 level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of PACA1 and/or PACA3 levels in samples derived from normal or healthy subjects of a similar age.

144. The method of any one of claims 135-143, wherein the normal or healthy subjects do not have lupus nephritis.

145. The method of any one of claims 137-144, wherein the elevated PACA1 and/or PACA3 level is greater than the reference PACA1 and/or PACA3 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

146. The method of any one of claims 137-144, wherein the elevated PACA1 and/or PACA3 level is greater than the reference PACA1 and/or PACA3 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

147. The method of claim 6, wherein the subject has an elevated C3a level.

148. The method of claim 147, wherein the subject has at least one of the following additional characteristics:
a. an elevated C4x/C4 ratio;
b. a reduced C4 level;
c. an elevated C1sC1 inhibitor level;
d. an elevated C1sC1 inhibitor/C1s ratio;
e. a reduced C1s level;
f. an elevated C2b level;
g an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C4x level;
k. an elevated C3a/C3 ratio; or
l. a reduced C3 level.

149. The method of any one of claims 6-148, wherein the elevated C3a level is greater than a C3a level in normal or healthy subjects.

150. The method of any one of claims 6-148, wherein the elevated C3a level is greater than a C3a level in normal or healthy subjects of a similar age.

151. The method of any one of claims 6-150, wherein the elevated C3a level is greater than a reference C3a level.

152. The method of claim 151, wherein the reference C3a level is a value that is equal to or greater than the mean or median of C3a levels in samples derived from lupus nephritis subjects.

153. The method of claim 151, wherein the reference C3a level is a value that is equal to or greater than the mean or median of C3a levels in samples derived from lupus nephritis subjects of a similar age.

154. The method of claim 151, wherein the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects.

155. The method of claim 151, wherein the reference C3a level is a value that is equal to or greater than the 55th percentile, 60th percentile, 65th percentile, 70th percentile, 75th percentile, 80th percentile, 85th percentile, 90th percentile, 95th percentile, or 100th percentile of C3a level in samples derived from lupus nephritis subjects of a similar age.

156. The method of claim 151, wherein the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects.

157. The method of claim 151, wherein the reference C3a level is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a levels in samples derived from normal or healthy subjects of a similar age.

158. The method of any one of claims 149-157, wherein the normal or healthy subjects do not have lupus nephritis.

159. The method of any one of claims 151-158, wherein the elevated C3a level is greater than the reference C3a level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

160. The method of any one of claims 151-158, wherein the elevated C3a level is greater than the reference C3a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

161. The method of claim 6, wherein the subject has an elevated C3a/C3 ratio.

162. The method of claim 161, wherein the subject has at least one of the following additional characteristics:
a. an elevated C4x level;
b. a reduced C4 level;
c. an elevated C1sC1 inhibitor level;
d. an elevated C1sC1 inhibitor/C1s ratio;
e. a reduced C1s level;
f. an elevated C2b level;
g an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C4x/C4 ratio; or
l. a reduced C3 level.

163. The method of any one of claims 6-162, wherein the elevated C3a/C3 ratio is greater than a C3a/C3 ratio in normal or healthy subjects.

164. The method of any one of claims 6-162, wherein the elevated C3a/C3 ratio is greater than a C3a/C3 ratio in normal or healthy subjects of a similar age.

165. The method of any one of claims 6-164, wherein the elevated C3a/C3 ratio is greater than a reference C3a/C3 ratio.

166. The method of claim 165, wherein the reference C3a/C3 ratio is a value that is equal to or greater than the mean or median of C3a/C3 ratio in samples derived from lupus nephritis subjects.

167. The method of claim 165, wherein the reference C3a/C3 ratio is a value that is equal to or greater than the mean or median of C3a/C3 ratio in samples derived from lupus nephritis subjects of a similar age.

168. The method of claim 165, wherein the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects.

169. The method of claim 165, wherein the reference C3a/C3 ratio is a value that is equal to or greater than the 55th percentile, 60th percentile, 65th percentile, 70th percentile, 75th percentile, 80th percentile, 85th percentile, 90th percentile, 95th percentile, or 100th percentile of C3a/C3 ratio in samples derived from lupus nephritis subjects of a similar age.

170. The method of claim 165, wherein the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects.

171. The method of claim 165, wherein the reference C3a/C3 ratio is a value that is equal to or greater than the 50th percentile, 55th percentile, 60th percentile, 65th percentile, 70th percentile, 75th percentile, 80th percentile, 85th percentile, 90th percentile, 95th percentile, or 100th percentile of C3a/C3 ratio in samples derived from normal or healthy subjects of a similar age.

172. The method of any one of claim 163-171, wherein the normal or healthy subjects do not have lupus nephritis.

173. The method of any one of claims 165-172, wherein the elevated C3a/C3 ratio is greater than the reference C3a/C3 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

174. The method of any one of claims 165-172, wherein the elevated C3a/C3 ratio is greater than the reference C3a/C3 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

175. The method of claim 6, wherein the subject has a reduced C3 level.

176. The method of claim 175, wherein the subject has at least one of the following additional characteristics:
a. an elevated C4x level;
b. an elevated C4x/C4 ratio;
c. an elevated C1sC1 inhibitor level;
d. an elevated C1sC1 inhibitor/C1s ratio;
e. a reduced C1s level;
f. an elevated C2b level;
g. an elevated C2b/C2 ratio;
h. a reduced C2 level;
i. an elevated Pathogenic Anti-C1q Antibody 1 (PACA1) and/or Pathogenic Anti-C1q Antibody 3 (PACA3) level;
j. an elevated C3a level;
k. an elevated C3a/C3 ratio, or
l. a reduced C4 level.

177. The method of any one of claims 6-176, wherein the reduced C3 level is less than a C3 level in normal or healthy subjects.

178. The method of any one of claims 6-176, wherein the reduced C3 level is less than a C3 level in normal or healthy subjects of a similar age.

179. The method of any one of claims 177-178, wherein the reduced C3 level is less than a reference C3 level.

180. The method of claim 179, wherein the reference C3 level is a value that is equal to or less than the mean or median of C3 levels in samples derived from lupus nephritis subjects.

181. The method of claim 179, wherein the reference C3 level is a value that is equal to or less than the mean or median of C3 levels in samples derived from lupus nephritis subjects of a similar age.

182. The method of claim 179, wherein the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects.

183. The method of claim 179, wherein the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 level in samples derived from lupus nephritis subjects of a similar age.

184. The method of claim 179, wherein the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects.

185. The method of claim 179, wherein the reference C3 level is a value that is equal to or less than the 55th percentile, 50th percentile, 45th percentile, 40th percentile, 35th percentile, 30th percentile, 25th percentile, 20th percentile, 15th percentile, 10th percentile, 5th percentile, or 0th percentile of C3 levels in samples derived from normal or healthy subjects of a similar age.

186. The method of any one of claims 177-175, wherein the normal or healthy subjects do not have lupus nephritis.

187. The method of any one of claims 179-186, wherein the reduced C3 level is less than the reference C4 level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

188. The method of any one of claims 179-186, wherein the reduced C3 level is less than the reference C4 level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

189. The method of any one of claims 1-188, wherein the C4x level, or the C4x/C4 ratio, or the C4 level, or the C1sC1 inhibitor level, or the C1sC1 inhibitor/C1s ratio, or the C1s level, or the C2b level, or the C2b/C2 ratio, or the C2 level, or the PACA1 and/or PACA3 level or the C3a level, or the C3a/C3 ratio, or the C3 level is measured in plasma or urine.

190. The method of any one of claims 1-189, wherein the subject has an elevated urine protein/creatinine ratio (UPCR) level.

191. The method of claim 190, wherein the elevated UPCR level is greater than a UPCR level in normal or healthy subjects.

192. The method of claim 190, wherein the elevated UPCR level is greater than a UPCR level in normal or healthy subjects of a similar age.

193. The method of any one of claims 190-192, wherein the elevated UPCR level is greater than a reference UPCR level.

194. The method of claim 193, wherein the reference UPCR level is equal to or greater than about 0.5 g/g, 1.0 g/g, 1.5 g/g, 2.0 g/g, 2.5 g/g, 3.0 g/g, 3.5 g/g, 4.0 g/g, 4.5 g/g, or 5.0 g/g.

195. The method of any one of claims 192-194, wherein the elevated UPCR level is greater than the UPCR level in normal or healthy subjects or the reference UPCR level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000%, 4000%, or 5000%.

196. The method of any one of claims 192-194, wherein the elevated UPCR level is greater than the UPCR level in normal or healthy subjects or the reference UPCR level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%.

197. The method of any one of claims 191-196, wherein the UPCR level is measured in urine.

198. The method of any one of claims 1-197, wherein the classical complement inhibitor is a C1 inhibitor.

199. The method of claim 198, wherein the C1 inhibitor is a small molecule, an antibody, an aptamer, an antisense nucleic acid or a gene editing agent.

200. The method of any one of claims 1-197, wherein the inhibitor of the classical complement pathway is a C1q inhibitor.

201. The method of claim 200, wherein the C1q inhibitor is a small molecule, an antibody, an aptamer, an antisense nucleic acid or a gene editing agent.

202. The method of claim 201, wherein the antibody is an anti-C1q antibody.

203. The method of claim 202, wherein the antibody is a monoclonal antibody, a polyclonal antibody, a recombinant antibody, a humanized antibody, a chimeric antibody, a multispecific antibody, or an antibody derivative thereof.

204. The method of claim 202, wherein the antibody derivative thereof is a single arm antibody.

205. The method of claim 203 or 204, wherein the antibody is administered at a dose of at least 50 mg/kg.

206. The method of claim 203 or 204, wherein the antibody is administered at a dose between 50 mg/kg to 200 mg/kg.

207. The method of claim 203 or 204, wherein the antibody is administered at a dose of 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, or 200 mg/kg.

208. The method of any one of claims 203-207, wherein the antibody is administered to a total of at least 50 mg.

209. The method of any one of claims 203-207, wherein the antibody is administered to a total dose of 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg.

210. The method of any one of claims 207-209, wherein the antibody is administered daily.

211. The method of any one of claims 207-209, wherein the antibody is administered once a week.

212. The method of any one of claims 207-209, wherein the antibody is administered once every other week.

213. The method of any one of claims 207-209, wherein the antibody is administered once a month.

214. The method of any one of claims 207-209, wherein the antibody is administered once every six weeks.

215. The method of any one of claims 207-209, wherein the antibody is administered once every other month.

216. The method of any one of claims 207-209, wherein the antibody is administered for at least 3 months, 6 months, at least 7 months, at least 8 months, at least 9 months, at least 10 months, at least 11 months, or at least 12 months.

217. The method of claim 216, wherein the antibody is administered for 6 months.

218. The method of any one of claims 207-209, wherein the antibody is administered throughout lifetime of the patient or intermittently throughout the lifetime of the patient in response to a flare.

219. The method of claim 203 or 204, wherein the antibody is administered at a dose of 75 mg/kg on day 1 and on day 5 or day 6.

220. The method of claim 219, wherein the antibody is further administered at a dose of 100 mg/kg every two weeks.

221. The method of any one of claims 203-220, wherein the antibody is administered intravenously.

222. The method of claim 202, wherein the antibody is an antibody fragment.

223. The method of claim 221, wherein the antibody fragment is a Fab fragment, a Fab′ fragment, a F(ab′)2 fragment, a Fv fragment, a diabody, or a single chain antibody molecule.

224. The method of claim 222 or 223, wherein the antibody fragment is administered to a total dose of at least 250 mg.

225. The method of claim 222 or 223, wherein the antibody fragment is administered to a total dose between 250 mg to 1000 mg.

226. The method of any one of claims 222-225, wherein the antibody fragment is administered to a total dose of 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg.

227. The method of any one of claims 222-226, wherein the antibody fragment is administered daily.

228. The method of any one of claims 222-226, wherein the antibody fragment is administered once every other day.

229. The of any one of claims 222-226, wherein the antibody fragment is administered once every three days, once every four days, once every five days, or once every six days.

230. The method of any one of claims 222-226, wherein the antibody fragment is administered once a week.

231. The method of any one of claims 222-226, wherein the antibody fragment is administered once every two weeks.

232. The method of any one of claims 222-226, wherein the antibody fragment is administered once a month.

233. The method of claim 222-226, wherein the antibody fragment is administered for at least 3 months, 6 months, at least 7 months, at least 8 months, at least 9 months, at least 10 months, at least 11 months, or at least 12 months.

234. The method of any one of claims 222-226 wherein the antibody fragment is administered throughout lifetime of the patient or intermittently throughout lifetime of the patient upon flare.

235. The method of any one of claims 298-234, wherein the antibody fragment is administered subcutaneously.

236. The method of any one of claims 198-235, wherein the anti-C1q antibody inhibits the interaction between C1q and an autoantibody or between C1q and C1r, or between C1q and C1s.

237. The method of any one of claims 202-235, wherein the anti-C1q antibody promotes clearance of C1q from circulation or a tissue.

238. The method of any one of claims 202-237, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7.

239. The method of any one of claims 202-238, wherein the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

240. The method of any one of claims 202-239, wherein the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7.

241. The method of claim 240, wherein the light chain variable domain comprising an amino acid sequence selected from SEQ ID NO: 4 and 35-38.

242. The method of any one of claims 202-241, wherein the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11.

243. The method of claim 242, wherein the heavy chain variable domain comprising an amino acid sequence selected from SEQ ID NO: 8 and 31-34.

244. The method of any one of claims 198-243, wherein the antibody fragment comprises heavy chain Fab fragment of SEQ ID NO: 39 and light chain Fab fragment of SEQ ID NO: 40.

245. The method of any one of claims 1-197, wherein the inhibitor of the classical complement pathway is a C1r inhibitor.

246. The method of claim 245, wherein the C1r inhibitor is a small molecule, an antibody, an aptamer, an antisense nucleic acid or a gene editing agent.

247. The method of claim 246, wherein the antibody is an anti-C1r antibody.

248. The method of claim 247, wherein the anti-C1r antibody inhibits the interaction between C1r and C1q or between C1r and C1s, or wherein the anti-C1r antibody inhibits the catalytic activity of C1r or inhibits the processing of pro-C1r to an active protease.

249. The method of any one of claims 1-197, wherein the inhibitor of the classical complement pathway is a C1s inhibitor.

250. The method of claim 249, wherein the C1s inhibitor is a small molecule, an antibody, an aptamer, an antisense nucleic acid or a gene editing agent.

251. The method of claim 250, wherein the antibody is an anti-C1s antibody.

252. The method of claim of 251, wherein the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s.

253. The method of claim 251, wherein the antibody is sutimlimab.

254. The method of any one of claims 1-197, wherein the inhibitor of the classical complement pathway is an anti-C1 complex antibody, optionally wherein the anti-C1 complex antibody inhibits C1r or C1s activation or blocks their ability to act on C2 or C4.

255. The method of claim 254, wherein the anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s.

256. The method of any one of claims 1-197, wherein the inhibitor of the classical complement pathway is a C2 inhibitor.

257. The method of claim 256, wherein the C2 inhibitor is a small molecule, an antibody, an aptamer, an antisense nucleic acid or a gene editing agent.

258. The method of any one of claims 1-197, wherein the inhibitor of the classical complement pathway is a C3 inhibitor.

259. The method of claim 258, wherein the C3 inhibitor is a small molecule, an antibody, an aptamer, an antisense nucleic acid or a gene editing agent.

260. The method of claim 259, wherein the C3 inhibitor is APL-9 (Apellis) or AMY-101 (Amyndas).

261. The method of any one of claims 1-197, wherein the inhibitor of the classical complement pathway is a C4 inhibitor.

262. The method of claim 261, wherein the C4 inhibitor is a small molecule, an antibody, an aptamer, an antisense nucleic acid or a gene editing agent.
